(19) United States
(12) Patent Application Publication
(10) Pub. No.: US 2011/0262347 A1
(43) Pub. Date: Oct. 27, 2011

(54) METHODS AND COMPOSITIONS FOR ENHANCED DELIVERY OF COMPOUNDS

(75) Inventors: Erkki Ruoslahti, Buellton, CA (US); Lilach Agemy, Santa Barbara, CA (US); Dinorah Friedmann-Morviniski, San Diego, CA (US); Venkata Ramana Kotamraju, Goleta, CA (US); Kazuki Sugahara, Goleta, CA (US); Inder Verma, La Jolla, CA (US)

(73) Assignees: The Salk Institute for Biological Studies; Sanford-Burnham Medical Research Institute

(21) Appl. No.: 13/083,176

(22) Filed: Apr. 8, 2011

Related U.S. Application Data
(60) Provisional application No. 61/322,207, filed on Apr. 8, 2010.
(60) Provisional application No. 61/376,856, filed on Aug. 25, 2010.

(51) Int. Cl. A61K 51/00; C07K 7/08; C07K 14/76; A61K 47/42; A61K 9/127; C07F 13/00; C07H 21/00; A61K 38/16; A61K 31/7088; A61K 31/28; C07D 305/14; A61K 31/337; A61K 49/00; A61P 35/00; A61P 43/00; C07K 7/06; B82Y 5/00

(52) U.S. Cl. 424/1.11; 530/330; 530/329; 530/327; 530/326; 530/362; 514/773; 424/450; 534/14; 536/23.1; 514/19.3; 514/44 R; 514/492; 549/510; 514/449; 424/9.1; 977/700; 977/811; 977/838

(57) ABSTRACT

Disclosed are compositions and methods related to multivalent compositions targeted to cells and tissues. The disclosed targeting is useful for treatment of cancer and other diseases and disorders.

### CROSS-REFERENCE TO RELATED APPLICATIONS

This application claims benefit of U.S. Provisional Application No. 61/322,207, filed Apr. 8, 2010, and U.S. Provisional Application No. 61/376,856, filed Aug. 25, 2010. Application No. 61/322,207, filed Apr. 8, 2010, and Application No. 61/376,856, filed Aug. 25, 2010, are hereby incorporated herein by reference in their entirety.

### STATEMENT REGARDING FEDERALLY SPONSORED RESEARCH

This invention was made with government support under Grant Nos. 5 P30 CA 30199-28 and P01 CA 104898-01 awarded by the National Institutes of Health (NIH), and DOD/USAMRAA Grant No. PC 093283 awarded by the Department of Defense (DOD). The government has certain rights in this invention.

### FIELD OF THE INVENTION

The present invention relates generally to the field of molecular medicine, and, more specifically, to compositions that home to targeted cells and tissue.

### BACKGROUND OF THE INVENTION

A major hurdle to advances in treating cancer is the relative lack of agents that can selectively target the cancer while sparing normal tissue. For example, radiation therapy and surgery, which generally are localized treatments, can cause substantial damage to normal tissue in the treatment field, resulting in scarring and loss of normal tissue. Chemotherapy, in comparison, which generally is administered systemically, can cause substantial damage to organs such as the bone marrow, mucosae, skin and small intestine, which undergo rapid cell turnover and continuous cell division. As a result, undesirable side effects such as nausea, loss of hair and drop in blood cell count often occur when a cancer patient is treated intravenously with a chemotherapeutic drug. Such undesirable side effects can limit the amount of a drug that can be safely administered, thereby hampering survival rate and impacting the quality of patient life.

Nanomedicine is an emerging field that uses nanoparticles to facilitate the diagnosis and treatment of diseases. Notable early successes in the clinic include the use of superparamagnetic nanoparticles as a contrast agent in MRI and nanoparticle-based treatment systems (Desai 2006; Weissleder 1995). The first generation of nanoparticles used in tumor treatments rely on “leakiness” of tumor vessels for preferential accumulation in tumors; however, this enhanced permeability and retention (EPR) is not a constant feature of tumor vessels (Sinek 2004) and even when present, still leaves the nanoparticles to negotiate the high interstitial fluid pressure in tumors (Sinek 2004; Boucher 1990). An attractive alternative is to target nanoparticles to specific molecular receptors in the blood vessels because they are readily available for binding from the blood stream and because tumor vessels express a wealth of molecules that are not significantly expressed in the vessels of normal tissues (Hoffman 2003; Oh 2004; Ruoslahti 2002).

Glioblastomas multiforme (GBM) are the most common and lethal form of intracranial tumors. They account for approximately 70% of the 22,500 new cases of malignant primary brain tumors that are diagnosed in adults in the United States each year. Although relatively uncommon, malignant gliomas are associated with disproportionately high morbidity and mortality (median survival is only 12 to 15 months). Malignant gliomas are among the most vascular of human tumors. However, gliomas are among the most difficult cancers to treat. Their location in the brain makes them inaccessible to numerous drugs and therapeutic compositions. Treatments that can effectively target gliomas are needed.

Specific targeting of nanoparticles to tumors has been accomplished in various experimental systems (DeNardo 2005; Akerman 2002; Cai 2006), but the efficiency of delivery is generally low. In nature, amplified homing is an important mechanism ensuring sufficient platelet accumulation at sites of vascular injury. It involves target binding, activation, platelet-platelet binding, and formation of a blood clot.

### BRIEF SUMMARY OF THE INVENTION

Disclosed are compositions and methods useful for delivering significant amounts of compounds of interest to targeted cells and tissues. The disclosed compositions and methods are useful, for example, to deliver to targeted cells and tissues an effective amount of compounds that are excessively toxic. For example, disclosed are compositions comprising a surface molecule, one or more homing molecules, and a plurality of cargo molecules. The cargo molecules can be, for example, excessively toxic molecules. The cargo molecules can be, for example, membrane perturbing molecules. As another example, disclosed are compositions comprising a surface molecule, one or more homing molecules, and a plurality of membrane perturbing molecules. Also disclosed are methods comprising, for example, administering to a subject the disclosed compositions.

The homing molecules can home to targets of interest, such as cells and tissues of interest. For example, the homing molecules can home to tumor vasculature. The homing molecules can selectively home to targets of interest, such as cells and tissues of interest. For example, the homing molecules can selectively homes to tumor vasculature. The composition can home to one or more of the sites to be targeted. The composition can be internalized in cells. The composition can penetrate tissue. The composition can be internalized into cells at the targeted site. The composition can penetrate tissue at the targeted site. The composition can, for example be internalized into cancer cells. The composition can, for example, penetrate tumor tissue. The composition can, for example, bind inside tumor blood vessels.

In some forms, one or more of the homing molecules can comprise the amino acid sequence CGKRK (SEQ ID NO:1) or a conservative derivative thereof, the amino acid sequence CRKDKC (SEQ ID NO:2) or a conservative derivative thereof, or a combination. In some forms, one or more of the homing molecule can comprise the amino acid sequence CGKRK (SEQ ID NO:1) or a conservative variant thereof. In some forms, one or more of the homing molecules can comprise the amino acid sequence CGKRK (SEQ ID NO:1). In some forms, one or more of the membrane perturbing molecules can comprise the amino acid sequence _{D}(KLAKLAK)₂ (SEQ ID NO:3) or a conservative variant thereof, (KLAKLAK)₂ (SEQ ID NO:3) or a conservative variant thereof, (KLAKKLA)₂ (SEQ ID NO:5) or a conservative variant thereof, (KAAKKAA)₂ (SEQ ID NO:6) or a conservative variant thereof, (KLGKKLG)₃ (SEQ ID NO:7) or a conservative variant thereof, or a combination. In some forms, one or more of the membrane perturbing molecules can comprise the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), (KLGKKLG)₃ (SEQ ID NO:7), or a combination. In some forms, one or more of the membrane perturbing molecules can comprise the amino acid sequence _{D}(KLAKLAK)₂ (SEQ ID NO:3) or a conservative variant thereof. In some forms, one or more of the membrane perturbing molecules can comprise the amino acid sequence _{D}(KLAKLAK)₂ (SEQ ID NO:3).

In some forms, the composition comprises a plurality of surface molecules, a plurality of homing molecules and a plurality of cargo molecules. In some forms, the composition comprises one or more surface molecules, a plurality of homing molecules and a plurality of cargo molecules. In some forms, the composition comprises a plurality of surface molecules, one or more homing molecules and a plurality of cargo molecules. In some forms, the composition comprises a plurality of surface molecules, a plurality of homing molecules and one or more cargo molecules. In some forms, the composition comprises one or more surface molecules, one or more homing molecules and a plurality of cargo molecules. In some forms, the composition comprises one or more surface molecules, a plurality of homing molecules and one or more cargo molecules. In some forms, the composition comprises a plurality of surface molecules, one or more homing molecules and one or more cargo molecules.

In some forms, the composition comprises a surface molecule, a plurality of homing molecules and a plurality of cargo molecules, wherein one or more of the homing molecules and one or more of the cargo molecules are associated with the surface molecule. In some forms, the composition comprises a surface molecule, a plurality of homing molecules and a plurality of cargo molecules, wherein a plurality of the plurality of homing molecules and a plurality of the plurality of cargo molecules are associated with the surface molecule. In some forms, the composition comprises a surface molecule, a plurality of homing molecules and a plurality of cargo molecules, wherein the homing molecules and the cargo molecules are associated with the surface molecule.

In some forms, the composition comprises a surface molecule, wherein the surface molecule is multivalent for homing molecules and cargo molecules. In some forms, the composition comprises a surface molecule, wherein the surface molecule is multivalent for homing molecules and comprises one or more cargo molecules. In some forms, the composition comprises a surface molecule, wherein the surface molecule is multivalent for cargo molecules and comprises one or more homing molecules. In some forms, the composition comprises a surface molecule, wherein the surface molecule is multivalent for conjugates, wherein one or more of the conjugates comprise one or more homing molecules and one or more cargo molecules. In some forms, the composition comprises a surface molecule, wherein the surface molecule is multivalent for conjugates, wherein one or more of the conjugates comprise a plurality of homing molecules and a plurality cargo molecules. In some forms, the composition comprises a surface molecule, wherein the surface molecule is multivalent for conjugates, wherein one or more of the conjugates comprise a homing molecule and a cargo molecule. In some forms, the composition comprises a surface molecule, wherein the surface molecule is multivalent for conjugates, wherein each of the conjugates comprises a plurality of homing molecules and a plurality cargo molecules. In some forms, the composition comprises a surface molecule, wherein the surface molecule is multivalent for conjugates, wherein each of the conjugates comprises a homing molecule and a cargo molecule.

In some forms, the composition comprises a surface molecule, wherein the surface molecule comprises one or more conjugates, wherein one or more of the conjugates comprise one or more homing molecules and one or more cargo molecules. In some forms, the composition comprises a surface molecule, wherein the surface molecule comprises one or more conjugates, wherein one or more of the conjugates comprise a plurality of homing molecules and a plurality cargo molecules. In some forms, the composition comprises a surface molecule, wherein the surface molecule comprises one or more conjugates, wherein one or more of the conjugates comprise a homing molecule and a cargo molecule. In some forms, the composition comprises a surface molecule, wherein the surface molecule comprises one or more conjugates, wherein each of the conjugates comprises a plurality of homing molecules and a plurality cargo molecules. In some forms, the composition comprises a surface molecule, wherein the surface molecule comprises one or more conjugates, wherein each of the conjugates comprises a homing molecule and a cargo molecule.

In some forms, one or more of the membrane perturbing molecules can be conjugated to one or more of the homing molecules. In some forms, one or more of the conjugated membrane perturbing molecules and homing molecules can be covalently coupled. In some forms, one or more of the covalently coupled membrane perturbing molecules and homing molecules can comprise fusion peptides. In some forms, the homing molecules can be conjugated with the surface molecule. In some forms, one or more of the conjugated homing molecules can be directly conjugated to the surface molecule. In some forms, one or more of the conjugated homing molecules can be indirectly conjugated to the surface molecule. In some forms, one or more of the homing molecules can be covalently coupled to the surface molecule. In some forms, one or more of the covalently coupled homing molecules can be directly covalently coupled to the surface molecule. In some forms, one or more of the covalently coupled homing molecules can be indirectly covalently coupled to the surface molecule. In some forms, the membrane perturbing molecules can be conjugated with the surface molecule. In some forms, one or more of the conjugated membrane perturbing molecules are directly conjugated to the surface molecule. In some forms, one or more of the conjugated membrane perturbing molecules can be indirectly conjugated to the surface molecule. In some forms, one or more of the membrane perturbing molecules can be covalently coupled to the surface molecule. In some forms, one or more of the covalently coupled membrane perturbing molecules can be directly covalently coupled to the surface molecule. In some forms, one or more of the covalently coupled membrane perturbing molecules can be indirectly covalently coupled to the surface molecule.

In some forms, the composition can further comprise one or more internalization elements. In some forms, one or more of the homing molecules can comprise one or more of the internalization elements. In some forms, one or more of the membrane perturbing molecules can comprise one or more of the internalization elements. In some forms, the surface molecule can comprise one or more of the internalization elements not comprised in either the homing molecules or the membrane perturbing molecules. In some forms, the composition can further comprise one or more tissue penetration elements. In some forms, one or more of the tissue penetration elements can be comprised in an internalization element. In some forms, the tissue penetration element can be a CendR element.

In some forms, the surface molecule can comprise a nanoparticle. In some forms, the surface molecule can comprise a nanoworm. In some forms, the surface molecule can comprise an iron oxide nanoworm. In some forms, the surface molecule can comprise an iron oxide nanoparticle. In some forms, the surface molecule can comprise an albumin nanoparticle. In some forms, the surface molecule can comprise a liposome. In some forms, the surface molecule can comprise a micelle. In some forms, the surface molecule comprises a phospholipid. In some forms, the surface molecule comprises a polymer. In some forms, the surface molecule can comprise a microparticle. In some forms, the surface molecule can comprise a fluorocarbon microbubble.

In some forms, the composition can comprise at least 100 homing molecules. In some forms, the composition can comprise at least 1000 homing molecules. In some forms, the composition can comprise at least 10,000 homing molecules. In some forms, the composition can comprise at least 100 membrane perturbing molecules. In some forms, the composition can comprise at least 1000 membrane perturbing molecules. In some forms, the composition can comprise at least 10,000 membrane perturbing molecules.

In some forms, one or more of the homing molecules can be modified homing molecules. In some forms, one or more of the homing molecules can comprise a methylated homing molecule. In some forms, one or more of the methylated homing molecules can comprise a methylated amino acid segment. In some forms, one or more of the membrane perturbing molecules can be modified membrane perturbing molecules. In some forms, one or more of the membrane perturbing molecules comprise a methylated membrane perturbing molecule. In some forms, one or more of the methylated membrane perturbing molecules comprise a methylated amino acid segment. In some forms, the amino acid sequence is N- or C-methylated in at least one position.

In some forms, the composition can further comprise one or more moieties. In some forms, the moieties can be independently selected from the group consisting of an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, an image contrast agent, a fluorophore, fluorescein, rhodamine, a radionuclide, indium-111, technetium-99, carbon-11, and carbon-13. In some forms, at least one of the moieties can be a therapeutic agent. In some forms, the therapeutic agent can be iRGD, RGD, Abraxane, paclitaxel, taxol, or a combination. In some forms, at least one of the moieties can be a detectable agent. In some forms, the detectable agent can be FAM.

In some forms, the composition can have a therapeutic effect. In some forms, the composition can reduce tumor growth. In some forms, the therapeutic effect can be a slowing in the increase of or a reduction of tumor burden. In some forms, the therapeutic effect can be a slowing of the increase of or reduction of tumor size. In some forms, the subject can have one or more sites targeted, wherein the composition can home to one or more of the sites targeted. In some forms, the subject can have a tumor, wherein the composition can have a therapeutic effect on the tumor.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a schematic diagram of an apoptotic cell.

FIGS. 2A, 2B and 2C show cytotoxicity of _{D}(KLAKLAK)₂CGKRK peptide in cell lines. FIGS. 2A and 2B show the cytotoxicity of _{D}(KLAKLAK)₂CGKRK peptide in human umbilical vein endothelial cells (HUVEC) (A) and T3 (B) cells. FIG. 2C shows the cytotoxicity of _{D}(KLAKLAK)₂CGKRK in U87 cells Cultured cells were treated CGKRK, or _{D}(KLAKLAK)₂, or _{D}(KLAKLAK)₂CGKRK peptide. The cells were incubated with peptide for 24 hrs and cell death was quantified by MTT assays (n=3). Statistical analyses were performed with Student's t-test. Error bars, s.e.m.

FIGS. 3A, 3B and 3C show cytotoxicity of _{D}(KLAKLAK)₂CGKRK conjugated with NW HUVEC cells. Cultured HUVEC cells were treated with non-targeted _{D}(KLAKLAK)₂ conjugated NW (D(KLAKLAK)₂), CREKA conjugated NW (CREKA), CGKRK conjugated NW (CGKRK), or CGKRK-_{D}(KLAKLAK)₂ conjugated NW (D(KLAKLAK)₂-CGKRK). The cells were incubated with NW for 48 hrs without washing (A and C) or the NW were washed after 20 min (B) and cell death was quantified by MTT assays (n=3). FIG. 3A used rapidly proliferating HUVEC cells while FIG. 3C used synchronized HUVEC cells. Statistical analyses were performed with Student's t-test. Error bars, s.e.m.

FIG. 4 shows cytotoxicity of CGKRK-_{D}(KLAKLAK)₂ conjugated with NW in T3 cells. Cultured T3 cells were treated with non-targeted _{D}(KLAKLAK)₂ conjugated NW (D(KLAKLAK)₂), CREKA conjugated NW (CREKA), CGKRK conjugated NW (CGKRK), or CGKRK-_{D}(KLAKLAK)₂ conjugated with NW (D(KLAKLAK)₂-CGKRK). The cells were incubated with NW for 48 hrs and cell death was quantified by MTT assay.

FIGS. 5A and 5B show cytotoxicity of _{D}(KLAKLAK)₂CGKRK conjugated with NW in U87 cells. Cultured U87 cells were treated with non-targeted _{D}(KLAKLAK)₂ conjugated NW (shown as KLAKLAK-NW on the graph), KAKEC (SEQ ID NO:135) conjugated NW (KAKEC-NW), CGKRK conjugated NW (CGKRK-NW), or CGKRK-_{D}(KLAKLAK)₂ conjugated with NW (CIMERA-NW). The cells were incubated with NW for 24 or 48 hrs and cell death was quantified by MTT assays. These results are almost the same results seen with U251 which had 50-60% cell viability.

FIG. 6 shows the IC50 of _{D}(KLAKLAK)₂CGKRK peptide versus peptide on nanoworms. NW coated with _{D}(KLAKLAK)₂CGKRK via a 5-kDa PEG-linker were cleaved from the particles using DTT and the amount of peptide present on the particle was calculated to compare the amount of free peptide versus the peptide coated nanoparticle IC50 values.

FIG. 7 shows _{D}(KLAKLAK)₂CGKRK conjugated with NW induced apoptosis in HUVEC cells. HUVEC cells were left untreated (Control) or treated for 24, 48 and 72 hrs with an irrelevant peptide-NW (CREKA-NR; SEQ ID NO:92) or the _{D}(KLAKLAK)₂CGKRK-NR. Cells were incubated with Annexin V-PE in a buffer containing 7-Amino-actinomycin (7-AAD) and analyzed by flow cytometry. The percentage of Annexin V positive cells (apoptotic cells plus end stage apoptosis or already dead cells) is indicated in each graph.

FIG. 8 shows _{D}(KLAKLAK)₂CGKRK conjugated with NW induced apoptosis in T3 cells. T3 cells (tumor endothelial cells) were left untreated (Control) or treated for 24 and 48 hrs with an irrelevant peptide-NW (CREKA-NR; SEQ ID NO:92) or the D(KLAKLAK)₂CGKRK-NR. Cells were incubated with Annexin V-PE in a buffer containing 7-Amino-actinomycin (7-AAD) and analyzed by flow cytometry. The percentage of Annexin V positive cells (apoptotic cells plus end stage apoptosis or already dead cells) is indicated in each graph.

FIG. 9 shows _{D}(KLAKLAK)₂CGKRK conjugated with NW inhibits HUVEC capillary-like tube formation in vitro. Primary HUVECs were plated on growth factor reduced matrigel in 5% FCS medium alone (control), or containing CGKRK-NW (SEQ ID NO:92) (10 microg/ml), or containing _{D}(KLAKLAK)₂CGKRK-NW (5 and 10 microg/ml). The formation of networks of capillary-like structures was viewed by phase contrast-microscopy at 40× magnification 24 h after plating.

FIG. 10 shows caspase activity by HUVEC cells treated with _{D}(KLAKLAK)₂CGKRK-NW. Caspase-3 activity was determined in HUVEC cells 24 h after treatment with 3 or 10 microgram _{D}(KLAKLAK)₂CGKRK-NW using a caspase-Glo 3/7 assay kit. Two hours after reagent was added luminescence was recorded on luminometer.

FIG. 11 is a diagram of the glioblastomas multiforme (GBM) treatment with CGKRK-_{D}(KLAKLAK)₂-NW nanoworms (EXP NUMBER 1). Mice bearing RAS-sip53 induced brain tumors (three weeks post-injection) were intravenously injected with NW coated with peptides through a 5-kDa polyethylene glycol spacer. The particles were administered every other day for 14 days (5 mg iron/kg/day, total cumulative dose 35 mg/kg). Survival was monitored over time (n=3 per group).

FIG. 12 shows GBM treatment with CGKRK-_{D}(KLAKLAK)₂-NW nanoworms (EXP NUMBER 1). Mice bearing RAS-sip53 induced brain tumors (three weeks post-injection) were intravenously injected with NW coated with peptides through a 5-kDa polyethylene glycol spacer. The particles were administered every other day for 14 days (5 mg iron/kg/day, total cumulative dose 35 mg/kg). Survival was monitored over time (n=3 per group).

FIGS. 13A and 13B show GBM treatment with CGKRK-_{D}(KLAKLAK)₂-NW nanoworms (EXP NUMBER 2). Mice bearing RAS-sip53 induced brain tumors (injection to the right hippocampus) were intravenously injected with NW coated with peptides through a 5-kDa polyethylene glycol spacer. The particles alone or co-injection with iRGD were administered once a week for 6 weeks (one weeks post-viral injection) or every other day for two weeks and a half weeks (three weeks post-viral injection). All mice were monitored for luciferase signal using the IVIS system (the lentivector contains the luciferase reporter), only one representative mouse from the indicated groups is shown in the figure. Survival of the mice is being currently recorded (n=3 per group).

FIG. 14 shows ALT (L-Alanine-2-Oxoglutarate Aminotransferase) levels in mice pre and post-nanoworm treatment. Mice were bled one day before starting the treatment and one day following the two and a half treatment course. For the groups of mice injected every other day another blood collection was performed two weeks after the last day of treatment. The levels of ALT were tested in the serum of all the mice. Normal values go from 10-40 U/L.

FIGS. 15A, 15B and 15C show the GBM treatment with CGKRK-_{D}(KLAKLAK)₂-NW nanoworms. Panel A shows a schematic of the experiment. Mice bearing 005 brain tumor cells (10 day post-injection) were intravenously injected with NW coated with peptides through a 5-kDa polyethylene glycol spacer. The particles without and co-injection with iRGD were administered every other day for 14 days (5 mg iron/kg/day, total cumulative dose 35 mg/kg). Panel B shows a graph of survival. Survival was monitored over time (n=3 per group). Panel C shows the results of mice having tumors induced by injecting 3×105 005 cells into the right hippocampus area. The 005 cell line was derived from a lentivirally (RAS-sip53) induced brain tumor (3). Ten days after the tumor cell injection, the mice were intravenously injected with NR. The NWs were administered every other day for 14 days followed by one week gap and continued treatment for 14 days. All but 2 control mice have died of the tumors, whereas all of mice treated with CGKRK-D[KLAKLAK]2-NW are alive (top line) with no overt signs of a tumor (n=8 per group). The controls were: no NW (bottom line with about 15% survival at day 35), D[KLAKLAK]2-NW middle line with about 40% survival at day 35), and CGKRK-NW (middle line with about 50% survival at day 35).

FIG. 16 shows the structure of targeted theranostic NR. Aminated NW were synthesized according to Park et al (4) and reacted with NHS-PEG(5K)-maleimide. Subsequently, peptides were coated on the NW through reaction between the maleimide group on the PEG and a cysteine thiol of the peptide. Coupling through the side chain of the central cysteine in the D(KLAKLAK)₂CGKRK peptide gives the V-shaped structure depicted in the figure.

FIG. 17 is a graph of FAM-CGKRK peptide binding to mitochondria in the presence of unlabeled peptide (left panel) and a control peptide (right panel). FAM-CGKRK was incubated with purified mitochondria in the presence of increasing concentrations of either unlabeled CGKRK or an unrelated peptide (CREKA; SEQ ID NO:92) as a control.

FIG. 18 is a graph of phage binding to mitochondria. CGKRK phage and CREKA (SEQ ID NO:92) phage (as a control) were incubated with purified mitochondria. Titration of bound phage shows about 80 times more binding of the CGKRK phage than the control. Student's t-test (c), Error bars, mean±SD; n.s., **p<0.01; ***p<0.001.

FIG. 19 is a graph of adsorption (A₄₅₀ nm) versus biotin-CGKRK concentration (μM). Binding of increasing amounts of biotin-labeled CGKRK peptide to immobilized p32 protein was detected with streptavidin coupled to horseradish peroxidase and normalized to nonspecific binding in the absence of p32. The affinity of the peptide for p32 calculated from the binding curves is also shown. The saturation curve shown is average of three independent experiments. Error bars, mean±SD.

FIGS. 20A and 20B are graphs of the percent of inhibition versus non-labeled peptide added. FIG. 20A shows the results for biotin labeled CGKRK and FIG. 20B shows the results for biotin labeled LyP-1 peptide.

FIG. 21 shows Annexin V positive cells (%) when treated with various peptide compositions 24, 48 and 72 hours. HUVEC and T3 cells were left untreated (Control) or treated with a concentration of 10 μg/ml of NWs coated with either a control peptide (CREKA; SEQ ID NO:92), _{D}[KLAKLAK]₂, or CGKRK_{D}[KLAKLAK]₂. The cells were stained with Annexin and analyzed by flow cytometry. The total percentage of Annexin-positive cells (apoptotic and dead cells) is indicated.

FIG. 22 shows Annexin V positive cells (%) when treated with various peptide compositions for 30 minutes (when the particles were washed away) and the incubation was continued for 72 hrs. The cells were stained with Annexin and analyzed by flow cytometry. The total percentage of Annexin-positive cells (apoptotic and dead cells) is indicated.

FIG. 23 shows survival (%) versus time (days) for mice bearing lenti-viral (H-RasV12-sip53) induced brain tumors treated with _{D}[KLAKLAK]₂-NWs or CGKRK _{D}[KLAKLAK]₂-NWs. Mice bearing lenti-viral (H-RasV12-sip53) induced brain tumors in the right hippocampus were intravenously injected with NW coated with peptides. The particles were administered every other day for 18 days, starting 3 weeks post-viral injection. Survival curve of the non-treated and treated mice (n=8-10 per group).

FIG. 24 shows survival (%) versus time (days) for mice bearing 005 tumor cells treated with _{D}[KLAKLAK]₂-NWs, CGKRK-NWs, and CGKRK_{D}[KLAKLAK]₂-NWs. Tumors were developed by transplanting 3×10⁵ 005 cells into the right hippocampus of NOD-SCID mice. Ten days post-tumor cell transplantation, the mice were intravenously injected with NWs. The NWs (5 mg of iron/kg) were administered every other day for 3 weeks or administered non stop for the same period of time (n=8 per group). Survival curves of the treated mice are shown.

FIG. 25 shows survival (%) versus time (days) for mice bearing 005 tumor cells treated with CGKRK_{D}[KLAKLAK]₂-NWs with co-administration of cRGD or iRGD. Mice bearing orthotopic 005 tumors implanted 10 days earlier received every other day for 3 weeks intravenous injections of CGKRK _{D}[KLAKLAK]₂-NWs (5 mg of iron/kg) mixed with 4 mmol/kg of cRGD or iRGD. Results for control mice and mice administered only iRGD are also shown. Survival curves are shown (n=8-10 per group).

FIG. 26 shows inhibition of CGKRK peptide binding to p32 by anti-p32. Biotin-CGKRK at 1 μg/ml was incubated in microtiter wells coated with purified p32, and the binding was detected with streptavidin coupled to horseradish peroxidase and normalized to nonspecific binding in the absence of p32. The anti-32 antibody was prepared against the full-length p32 protein (Protein Production and Analysis Facility of the Sanford-Burnham Medical Research Institute). The experiments were performed in triplicate; one of two experiments with similar results is shown.

FIGS. 27A, 27B, and 27C shows that CGKRK_{D}[KLAKLAK]₂-NW conjugates induce cell death by apoptosis. HUVEC (A) and T3 (B) cells were left untreated (Control) or were treated with 10 μg/ml of NWs coated with CGKRK _{D}[KLAKLAK]₂-NWs for 48 (A) or 72 hours (B). In 27C, the cells were incubated with the indicated NWs, washed to remove excess NWs after 30 minutes, and then incubated for 72 hours. Annexin staining and analysis by flow cytometry were used to measure apoptosis in the cultures. Representative images are shown indicating the percentage of Annexin-positive cells (apoptotic and dead cells).

FIG. 28 shows toxicology analyses of mice treated with CGKRK_{D}[KLAKLAK]₂-NWs. Blood L-alanine-2-oxoglutarate aminotransferase (ALT) levels measured before (Pre-treatment), after completion of a 3-week treatment course (After treatment), and after a subsequent 2-week recovery period (2 weeks after treatment) are shown.

FIGS. 29A and 29B show toxicology analyses of mice treated with CGKRK_{D}[KLAKLAK]₂-NWs. Possible active and innate immune responses against NW was tested by measuring antibody (29A) and IL-6 levels (29B) in serum form mice treated and collected as in FIG. 28.

FIG. 30 shows survival curves of mice bearing intracranial U87 tumors treated with CGKRK_{D}[KLAKLAK]₂-NWs. Tumors were induced by injecting 5×10⁵ GFP-expressing U87 cells into the right hippocampus of mice. Treatment with intravenous injections of CGKRK-_{D}[KLAKLAK]₂-NWs and control NWs was started 10 days after the tumor cell injection and continued every other day for 3 weeks (n=5 per group).

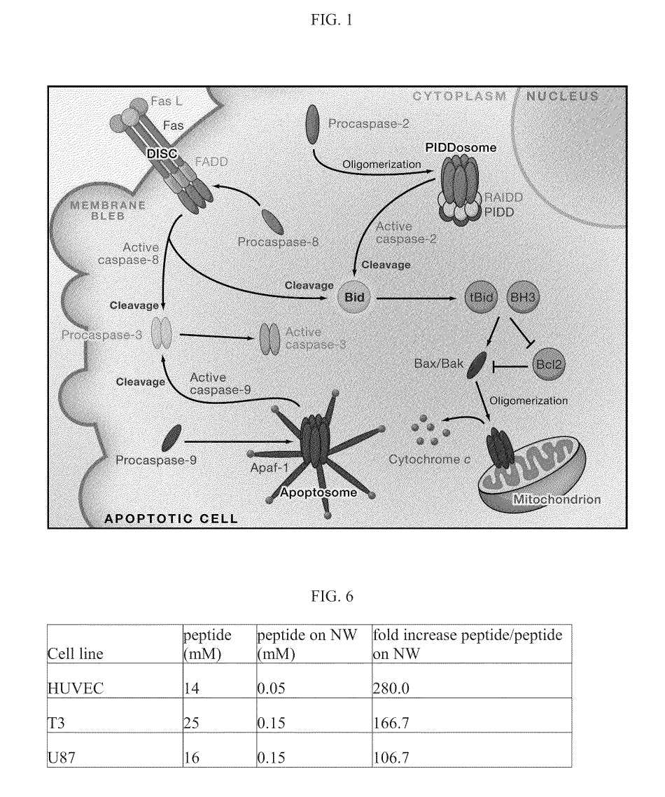

### DETAILED DESCRIPTION OF THE INVENTION

The disclosed methods and compositions can be understood more readily by reference to the following detailed description of particular embodiments and the Example included therein and to the Figures and their previous and following description.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular peptide is disclosed and discussed and a number of modifications that can be made to a number of molecules including the peptide are discussed, specifically contemplated is each and every combination and permutation of the peptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed are compositions useful for delivering significant amounts of compounds of interest to targeted cells and tissues. The disclosed compositions are useful, for example, to deliver to targeted cells and tissues an effective amount of compounds that are excessively toxic. For example, disclosed are compositions comprising a surface molecule, one or more homing molecules, and a plurality of cargo molecules. The cargo molecules can be, for example, excessively toxic molecules. The cargo molecules can be, for example, membrane perturbing molecules. As another example, disclosed are compositions comprising a surface molecule, one or more homing molecules, and a plurality of membrane perturbing molecules. As used herein, excessively toxic compounds are compounds that too toxic when administered to a subject in unconjugated forms in what would be a therapeutically effective amount but for the toxicity.

The homing molecules can home to targets of interest, such as cells and tissues of interest. For example, the homing molecules can home to tumor vasculature. The homing molecules can selectively home to targets of interest, such as cells and tissues of interest. For example, the homing molecules can selectively home to tumor vasculature. The composition can home to one or more of the sites to be targeted. The composition can be internalized in cells. The composition can penetrate tissue. The composition can be internalized into cells at the targeted site. The composition can penetrate tissue at the targeted site. The composition can, for example be internalized into cancer cells. The composition can, for example, penetrate tumor tissue. The composition can, for example, bind inside tumor blood vessels.

In some forms, one or more of the homing molecules can comprise the amino acid sequence CGKRK (SEQ ID NO:1) or a conservative derivative thereof, the amino acid sequence CRKDKC (SEQ ID NO:2) or a conservative derivative thereof, or a combination. In some forms, one or more of the homing molecule can comprise the amino acid sequence CGKRK (SEQ ID NO:1) or a conservative variant thereof. In some forms, one or more of the homing molecules can comprise the amino acid sequence CGKRK (SEQ ID NO:1). In some forms, one or more of the membrane perturbing molecules can comprise the amino acid sequence _{D}(KLAKLAK)₂ (SEQ ID NO:3) or a conservative variant thereof, (KLAKLAK)₂ (SEQ ID NO:3) or a conservative variant thereof, (KLAKKLA)₂ (SEQ ID NO:5) or a conservative variant thereof, (KAAKKAA)₂ (SEQ ID NO:6) or a conservative variant thereof, (KLGKKLG)₃ (SEQ ID NO:7) or a conservative variant thereof, or a combination. In some forms, one or more of the membrane perturbing molecules can comprise the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), (KLGKKLG)₃ (SEQ ID NO:7), or a combination. In some forms, one or more of the membrane perturbing molecules can comprise the amino acid sequence _{D}(KLAKLAK)₂ (SEQ ID NO:3) or a conservative variant thereof. In some forms, one or more of the membrane perturbing molecules can comprise the amino acid sequence _{D}(KLAKLAK)₂ (SEQ ID NO:3).

In some forms, the composition can comprise a plurality of surface molecules, a plurality of homing molecules and a plurality of cargo molecules. In some forms, the composition can comprise one or more surface molecules, a plurality of homing molecules and a plurality of cargo molecules. In some forms, the composition can comprise a plurality of surface molecules, one or more homing molecules and a plurality of cargo molecules. In some forms, the composition can comprise a plurality of surface molecules, a plurality of homing molecules and one or more cargo molecules. In some forms, the composition can comprise one or more surface molecules, one or more homing molecules and a plurality of cargo molecules. In some forms, the composition can comprise one or more surface molecules, a plurality of homing molecules and one or more cargo molecules. In some forms, the composition comprises a plurality of surface molecules, one or more homing molecules and one or more cargo molecules.

In some forms, the composition can comprise a surface molecule, a plurality of homing molecules and a plurality of cargo molecules, wherein one or more of the homing molecules and one or more of the cargo molecules are associated with the surface molecule. In some forms, the composition can comprise a surface molecule, a plurality of homing molecules and a plurality of cargo molecules, wherein a plurality of the plurality of homing molecules and a plurality of the plurality of cargo molecules are associated with the surface molecule. In some forms, the composition can comprise a surface molecule, a plurality of homing molecules and a plurality of cargo molecules, wherein the homing molecules and the cargo molecules are associated with the surface molecule.

In some forms, the composition can comprise a surface molecule, wherein the surface molecule is multivalent for homing molecules and cargo molecules. In some forms, the composition can comprise a surface molecule, wherein the surface molecule is multivalent for homing molecules and comprises one or more cargo molecules. In some forms, the composition can comprise a surface molecule, wherein the surface molecule is multivalent for cargo molecules and comprises one or more homing molecules. In some forms, the composition can comprise a surface molecule, wherein the surface molecule is multivalent for conjugates, wherein one or more of the conjugates comprise one or more homing molecules and one or more cargo molecules. In some forms, the composition can comprise a surface molecule, wherein the surface molecule is multivalent for conjugates, wherein one or more of the conjugates comprise a plurality of homing molecules and a plurality cargo molecules. In some forms, the composition can comprise a surface molecule, wherein the surface molecule is multivalent for conjugates, wherein one or more of the conjugates comprise a homing molecule and a cargo molecule. In some forms, the composition can comprise a surface molecule, wherein the surface molecule is multivalent for conjugates, wherein each of the conjugates comprises a plurality of homing molecules and a plurality cargo molecules. In some forms, the composition can comprise a surface molecule, wherein the surface molecule is multivalent for conjugates, wherein each of the conjugates comprises a homing molecule and a cargo molecule. As used herein, a component that is stated to be “multivalent for” one or more other components refers to a component that has a plurality of the other components associated with, conjugated to and/or covalent coupled to the first component.

In some forms, the composition can comprise a surface molecule, wherein the surface molecule comprises one or more conjugates, wherein one or more of the conjugates comprise one or more homing molecules and one or more cargo molecules. In some forms, the composition can comprise a surface molecule, wherein the surface molecule comprises one or more conjugates, wherein one or more of the conjugates comprise a plurality of homing molecules and a plurality cargo molecules. In some forms, the composition can comprise a surface molecule, wherein the surface molecule comprises one or more conjugates, wherein one or more of the conjugates comprise a homing molecule and a cargo molecule. In some forms, the composition can comprise a surface molecule, wherein the surface molecule comprises one or more conjugates, wherein each of the conjugates comprises a plurality of homing molecules and a plurality cargo molecules. In some forms, the composition can comprise a surface molecule, wherein the surface molecule comprises one or more conjugates, wherein each of the conjugates comprises a homing molecule and a cargo molecule.

In some forms, one or more of the membrane perturbing molecules can be conjugated to one or more of the homing molecules. In some forms, one or more of the conjugated membrane perturbing molecules and homing molecules can be covalently coupled. In some forms, one or more of the covalently coupled membrane perturbing molecules and homing molecules can comprise fusion peptides. In some forms, the homing molecules can be conjugated with the surface molecule. In some forms, one or more of the conjugated homing molecules can be directly conjugated to the surface molecule. In some forms, one or more of the conjugated homing molecules can be indirectly conjugated to the surface molecule. In some forms, one or more of the homing molecules can be covalently coupled to the surface molecule. In some forms, one or more of the covalently coupled homing molecules can be directly covalently coupled to the surface molecule. In some forms, one or more of the covalently coupled homing molecules can be indirectly covalently coupled to the surface molecule. In some forms, the membrane perturbing molecules can be conjugated with the surface molecule. In some forms, one or more of the conjugated membrane perturbing molecules are directly conjugated to the surface molecule. In some forms, one or more of the conjugated membrane perturbing molecules can be indirectly conjugated to the surface molecule. In some forms, one or more of the membrane perturbing molecules can be covalently coupled to the surface molecule. In some forms, one or more of the covalently coupled membrane perturbing molecules can be directly covalently coupled to the surface molecule. In some forms, one or more of the covalently coupled membrane perturbing molecules can be indirectly covalently coupled to the surface molecule.

In some forms, the composition can further comprise one or more internalization elements. In some forms, one or more of the homing molecules can comprise one or more of the internalization elements. In some forms, one or more of the membrane perturbing molecules can comprise one or more of the internalization elements. In some forms, the surface molecule can comprise one or more of the internalization elements not comprised in either the homing molecules or the membrane perturbing molecules. In some forms, the composition can further comprise one or more tissue penetration elements. In some forms, one or more of the tissue penetration elements can be comprised in an internalization element. In some forms, the tissue penetration element can be a CendR element.

In some forms, the surface molecule can comprise a nanoparticle. In some forms, the surface molecule can comprise a nanoworm. In some forms, the surface molecule can comprise an iron oxide nanoworm. In some forms, the surface molecule can comprise an iron oxide nanoparticle. In some forms, the surface molecule can comprise an albumin nanoparticle. In some forms, the surface molecule can comprise a liposome. In some forms, the surface molecule can comprise a micelle. In some forms, the surface molecule comprises a phospholipid. In some forms, the surface molecule comprises a polymer. In some forms, the surface molecule can comprise a microparticle. In some forms, the surface molecule can comprise a fluorocarbon microbubble.

In some forms, the composition can comprise at least 100 homing molecules. In some forms, the composition can comprise at least 1000 homing molecules. In some forms, the composition can comprise at least 10,000 homing molecules. In some forms, the composition can comprise at least 100 membrane perturbing molecules. In some forms, the composition can comprise at least 1000 membrane perturbing molecules. In some forms, the composition can comprise at least 10,000 membrane perturbing molecules.

In some forms, one or more of the homing molecules can be modified homing molecules. In some forms, one or more of the homing molecules can comprise a methylated homing molecule. In some forms, one or more of the methylated homing molecules can comprise a methylated amino acid segment. In some forms, one or more of the membrane perturbing molecules can be modified membrane perturbing molecules. In some forms, one or more of the membrane perturbing molecules comprise a methylated membrane perturbing molecule. In some forms, one or more of the methylated membrane perturbing molecules comprise a methylated amino acid segment. In some forms, the amino acid sequence is N- or C-methylated in at least one position.

In some forms, the composition can further comprise one or more moieties. In some forms, the moieties can be independently selected from the group consisting of an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, an image contrast agent, a fluorophore, fluorescein, rhodamine, a radionuclide, indium-111, technetium-99, carbon-11, and carbon-13. In some forms, at least one of the moieties can be a therapeutic agent. In some forms, the therapeutic agent can be iRGD, RGD, Abraxane, paclitaxel, taxol, or a combination. In some forms, at least one of the moieties can be a detectable agent. In some forms, the detectable agent can be FAM.

In some forms, the composition can have a therapeutic effect. In some forms, the composition can reduce tumor growth. In some forms, the therapeutic effect can be a slowing in the increase of or a reduction of tumor burden. In some forms, the therapeutic effect can be a slowing of the increase of or reduction of tumor size. In some forms, the subject can have one or more sites targeted, wherein the composition can home to one or more of the sites targeted. In some forms, the subject can have a tumor, wherein the composition can have a therapeutic effect on the tumor.

The disclosed components can be associated with each other (or, in some forms, not associated with each other) in combinations as disclosed herein. For example, homing molecules can be covalently coupled or non-covalently associated with surface molecules, homing molecules can be covalently coupled or non-covalently associated with membrane perturbing molecules, membrane perturbing molecules can be covalently coupled or non-covalently associated with surface molecules, etc. Associated components can also be referred to as being conjugated. Conjugation can be direct or indirect. Direct conjugation of components refers to covalently coupled or non-covalently associated components where there is no other molecule intervening between the conjugated components. Indirect conjugation refers to any chain of molecules and covalent bonds or non-covalent associations linking the components where the components are not directly conjugated (that is, there is a least one separate molecule other than the components intervening between the components).

Covalently coupled refers to association of components via covalent bonds. A covalent association or coupling can be either direct or indirect. A direct covalent association or coupling of components refers to a covalent bond involving atoms that are each respectively a part of the components. Thus, in a direct covalent association or coupling, there is no other molecule intervening between the associated/coupled components. An indirect covalent association or coupling refers to any chain of molecules and covalent bonds linking the components where the components are not covalently coupled (that is, there is a least one separate molecule other than the components intervening between the components via covalent bonds).

As used herein, reference to components (such as a homing molecule and a surface molecule) as being “not covalently coupled” means that the components are not connected via covalent bonds (for example, that the homing molecule and the surface molecule are not connected via covalent bonds). That is, there is no continuous chain of covalent bonds between, for example, the homing molecule and the surface molecule.

Non-covalent association refers to association of components via non-covalent bonds and interactions. A non-covalent association can be either direct or indirect. A direct non-covalent association refers to a non-covalent bond involving atoms that are each respectively connected via a chain of covalent bonds to the components. Thus, in a direct non-covalent association, there is no other molecule intervening between the associated components. An indirect non-covalent association refers to any chain of molecules and bonds linking the components where the components are not covalently coupled (that is, there is a least one separate molecule other than the components intervening between the components via non-covalent bonds).

Reference to components (such as a homing molecule and a surface molecule) as not being “non-covalently associated” means that there is no direct or indirect non-covalent association between the components. That is, for example, no atom covalently coupled to a homing molecule is involved in a non-covalent bond with an atom covalently coupled to a surface molecule. Within this meaning, a homing molecule and a surface molecule can be together in a composition where they are indirectly associated via multiple intervening non-covalent bonds while not being non-covalently associated as that term is defined herein. For example, a homing molecule and a surface molecule can be mixed together in a carrier where they are not directly non-covalently associated. A homing molecule and a surface molecule that are referred to as not indirectly non-covalently associated cannot be mixed together in a continuous composition. Reference to components (such as a homing molecule and a surface molecule) as not being “directly non-covalently associated” means that there is no direct non-covalent association between the components (an indirect non-covalent association may be present). Reference to components (such as a homing molecule and a surface molecule) as not being “indirectly non-covalently associated” means that there is no direct or indirect non-covalent association between the components.

It is understood that components can be non-covalently associated via multiple chains and paths including both direct and indirect non-covalent associations. For the purposes of these definitions, the presence a single direct non-covalent association makes the association a direct non-covalent association even if there are also indirect non-covalent associations present. Similarly, the presence of a covalent connection between components means the components are covalently coupled even if there are also non-covalent associations present. It is also understood that covalently coupled components that happened to lack any non-covalent association with each other are not considered to fall under the definition of components that are not non-covalently associated.

Association of the components of the disclosed compositions can be aided or accomplished via molecules, conjugates and/or compositions. Where such molecules, conjugates and/or compositions are other than surface molecules, homing molecules, or cargo molecules (such as membrane perturbing molecules, internalization elements, tissue penetration elements, and moieties), they can be referred to herein as linkers. Such linkers can be any molecule, conjugate, composition, etc. that can be used to associate components of the disclosed compositions. Generally, linkers can be used to associate components other than surface molecules to surface molecules. Useful linkers include materials that are biocompatible, have low bioactivity, have low antigenicity, etc. That is, such useful linker materials can serve the linking/association function without adding unwanted bioreactivity to the disclosed compositions. Many such materials are known and used for similar linking and association functions. Polymer materials are a particularly useful form of linker material. For example, polyethylene glycols can be used.

Linkers are useful for achieving useful numbers and densities of the components (such as homing molecules and membrane perturbing molecules) on surface molecules. For example, linkers of fibrous form are useful for increasing the number of components per surface molecule or per a given area of the surface molecule. Similarly, linkers having a branching form are useful for increasing the number of components per surface molecule or per a given area of the surface molecule. Linkers can also have a branching fibrous form.

Sufficiency of the number and composition of homing molecules in the composition can be determined by assessing homing to the target and effectively delivery of the cargo molecules in a non-human animal. The composition can comprise a sufficient number and composition of homing molecules (modified or not) such that the composition homes to the target and effectively delivers the cargo molecules. In one example, sufficiency of the number and composition of modified and/or unmodified homing molecules can be determined by assessing cargo delivery and/or therapeutic effect on the target. Sufficiency of the number and composition of membrane perturbing molecules can be determined by assessing membrane perturbing effect of the composition in a non-human animal. The composition can comprise a sufficient number and composition of membrane perturbing molecules (modified or not) such that the composition has a membrane perturbing effect on the target. In one example, sufficiency of the number and composition of modified and/or unmodified membrane perturbing molecules can be determined by assessing membrane disruption, apoptosis, and/or therapeutic effect on the target.

The composition can comprise a sufficient density and composition of homing molecules such that the composition homes to the target and effectively delivers the cargo molecules. Sufficiency of the density and composition of homing molecules can be determined by assessing cargo delivery and/or therapeutic effect on the target in a non-human animal. The composition can comprise a sufficient density and composition of membrane perturbing molecules such that the composition has a membrane perturbing effect on the target. Sufficiency of the density and composition of membrane perturbing molecules can be determined by assessing membrane disruption, apoptosis, and/or therapeutic effect on the target in a non-human animal.

The density of homing molecules and/or membrane perturbing molecules on a surface molecule can be described in any suitable manner. For example, the density can be expressed as the number of homing molecules and/or membrane perturbing molecules per, for example, a given area, surface area, volume, unit, subunit, arm, etc. of the surface molecule. The density can also be relative to, for example, the area, surface area, volume, unit, subunit, arm, etc. of the entire surface molecule or to the area, surface area, volume, unit, subunit, arm, etc. of a portion of the surface molecule. For example, a sufficient density of homing molecule and/or membrane perturbing molecule can be present in a portion of the surface molecule. The presence of this dense portion can cause clotting and amplify the accumulation of the composition. Thus, a composition having a sufficient density of homing molecules and/or membrane perturbing molecules can have a threshold density (or above) for the entire surface molecule or for just one or more portions of the surface molecule. Unless otherwise stated, densities refer to average density over the designated portion of the surface molecule. For example, a density of 1 homing molecule per square nM of the surface molecule refers to an average density of the homing molecules over the entire surface molecule. As another example, a density of 1 homing molecule per square nM of a portion of the surface molecule refers to an average density of the homing molecules over just that portion of the surface molecule.

The density can be measured or calculated in any suitable manner. For example, the number or amount of homing molecules and/or membrane perturbing molecules present on a surface molecule or group of surface molecules can be measured by, for example, detecting the level or intensity of signal produced by labeled homing molecules and/or membrane perturbing molecules and calculating the density based on the structural characteristics of the surface molecule.

The density or threshold density of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per square nM of the entire or a portion of the surface molecule. The composition can also comprise any density in between those densities listed above.

The density or threshold density of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per square μM of the entire or a portion of the surface molecule. The composition can also comprise any density in between those densities listed above.

The density or threshold density of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per cubic nM of the entire or a portion of the surface molecule. The composition can also comprise any density in between those densities listed above.

The density or threshold density of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per cubic μM of the entire or a portion of the surface molecule. The composition can also comprise any density in between those densities listed above.

The number of homing molecules and/or membrane perturbing molecules on a surface molecule can be described in any suitable manner. For example, the number can be expressed as the number of homing molecules and/or membrane perturbing molecules per, for example, a given area, surface area, volume, unit, subunit, arm, etc. of the surface molecule. The number can also be relative to, for example, the area, surface area, volume, unit, subunit, arm, etc. of the entire surface molecule or to the area, surface area, volume, unit, subunit, arm, etc. of a portion of the surface molecule. For example, a sufficient number of homing molecule and/or membrane perturbing molecule can be present in a portion of the surface molecule. The presence of this dense portion can cause clotting and amplify the accumulation of the composition. Thus, a composition having a sufficient number of homing molecules and/or membrane perturbing molecules can have a threshold number (or above) for the entire surface molecule or for just one or more portions of the surface molecule.

The number can be measured or calculated in any suitable manner. For example, the number or amount of homing molecules and/or membrane perturbing molecules present on a surface molecule or group of surface molecules can be measured by, for example, detecting the level or intensity of signal produced by labeled homing molecules and/or membrane perturbing molecules and calculating the number based on the structural characteristics of the surface molecule.

The number or threshold number of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules on the surface molecule. The composition can also comprise any number in between those numbers listed above.

The number or threshold number of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per square nM of the entire or a portion of the surface molecule. The composition can also comprise any number in between those numbers listed above.

The number or threshold number of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per square μM of the entire or a portion of the surface molecule. The composition can also comprise any number in between those numbers listed above.

The number or threshold number of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per cubic nM of the entire or a portion of the surface molecule. The composition can also comprise any number in between those numbers listed above.

The number or threshold number of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per cubic μM of the entire or a portion of the surface molecule. The composition can also comprise any number in between those numbers listed above.

In some forms, the compositions not only home to tumors, but also amplify their own homing. Homing molecules can be used that are clot-binding compounds that recognize clotted plasma proteins and selectively homes to tumors, where it binds to vessel walls and tumor stroma. Surface molecules coupled with the clot-binding compounds can accumulate in tumor vessels or at wound sites, where they induce additional local clotting, thereby producing new binding sites for more particles. The system mimics platelets, which also circulate freely but accumulate at a diseased site and amplify their own accumulation at that site. The clotting-based amplification greatly enhances cargo delivery and tumor imaging.

### A. Homing Molecules

Homing molecules allow the disclosed compositions to be targeted and to home to desired target sites. Homing molecules generally bind preferentially to target molecules, cells, tissues, etc., thus resulting in an accumulation of the homing molecules (and other components to which they are associated) at target sites.

The term “homing molecule” as used herein, means any molecule that selectively homes in vivo to specified target sites, such as cells or tissues, in preference to normal or other non-target sites, cells, or tissues. Similarly, the term “homing peptide” or “homing peptidomimetic” means a peptide that selectively homes in vivo to specified target sites, such as cells or tissues, in preference to normal or other non-target sites, cells, or tissues. It is understood that a homing molecule that selectively homes in vivo to, for example, tumors can home to all tumors or can exhibit preferential homing to one or a subset of tumor types.

By “selectively homes” it is meant that in vivo, the homing molecule binds preferentially to the target as compared to non-target. For example, the homing molecule can bind preferentially to certain molecules, proteins, cells, tissues, etc. as compared to other molecules, proteins, cells, tissues, etc. For example, the homing molecule can bind preferentially to tumor vasculature or one or more tumors as compared to non-tumoral tissue. Such a homing molecule can selectively home, for example, to tumors. Selective homing to, for example, certain molecules, proteins, cells, tissues, etc. generally is characterized by at least a two-fold greater localization the molecules, proteins, cells, tissues, etc. (or other target), as compared to other certain molecules, proteins, cells, tissues, etc. A homing molecule can be characterized by, for example, 5-fold, 10-fold, 20-fold or more preferential localization to the target as compared to one or more non-targets. For example, a homing molecule can be characterized by, for example, 5-fold, 10-fold, 20-fold or more preferential localization to tumor vasculature as compared to vasculature of several or many tissue types of non-tumoral tissue, or as compared to vasculature of most or all non-tumoral tissue. As another example, a homing molecule can be characterized by, for example, 5-fold, 10-fold, 20-fold or more preferential localization to tumors as compared to several or many tissue types of non-tumoral tissue, or as compared to-most or all non-tumoral tissue. Thus, it is understood that, in some cases, a homing molecule homes, in part, to one or more normal organs in addition to homing to the target tissue. Selective homing can also be referred to as targeting. The molecules, proteins, cells, tissues, etc. that are targeted by homing molecules can be referred to as targeted molecules, proteins, cells, tissues, etc.

In some forms, one or more of the homing molecules can comprise the amino acid sequence CGKRK (SEQ ID NO:1) or a conservative derivative thereof, the amino acid sequence CRKDKC (SEQ ID NO:2) or a conservative derivative thereof, or a combination. In some forms, one or more of the homing molecule can comprise the amino acid sequence CGKRK (SEQ ID NO:1) or a conservative variant thereof. In some forms, one or more of the homing molecules can comprise the amino acid sequence CGKRK (SEQ ID NO:1).

The composition can comprise a sufficient number and composition of homing molecules (modified or not) such that the composition homes to the target and effectively delivers the cargo molecules. In one example, sufficiency of the number and composition of modified and/or unmodified homing molecules can be determined by assessing cargo delivery and/or therapeutic effect on the target.

Many homing molecules and homing peptides home to the vasculature of the target tissue. However, for the sake of convenience homing is referred to in some places herein as homing to the tissue associated with the vasculature to which the homing molecule or homing peptide may actually home. Thus, for example, a homing molecule that homes to tumor vasculature can be referred to herein as homing to tumor tissue or to tumor cells. By including or associating a homing molecule or homing peptide with, for example, a protein, peptide, amino acid sequence, cargo molecules, or CendR element the protein, peptide, amino acid sequence, cargo molecules, or CendR element can be targeted or can home to the target of the homing molecule or homing peptide. In this way, the protein, peptide, amino acid sequence, cargo molecules, or CendR element can be said to home to the target of the homing molecule or homing peptide. For convenience and unless otherwise indicated, reference to homing of a protein, peptide, amino acid sequence, cargo molecules, CendR element, etc. is intended to indicate that the protein, peptide, amino acid sequence, cargo molecules, CendR element, etc. includes or is associated with an appropriate homing molecule or homing peptide.

The homing molecule can selectively home to a tumor. The homing molecule can selectively home to tumor vasculature. The homing molecule can selectively home to one or more particular types of tumor. The homing molecule can selectively home to the vasculature of one or more particular types of tumor. The homing molecule can selectively home to one or more particular stages of a tumor or cancer. The homing molecule can selectively home to the vasculature of one or more particular stages of a tumor or cancer. The homing molecule can selectively home to one or more particular stages of one or more particular types of tumor. The homing molecule can selectively home to the vasculature of one or more different stages of one or more particular types of tumor.

The composition can selectively home to a tumor. The composition can selectively home to tumor vasculature. The composition can selectively home to one or more particular types of tumor. The composition can selectively home to the vasculature of one or more particular types of tumor. The composition can selectively home to one or more particular stages of a tumor or cancer. The composition can selectively home to the vasculature of one or more particular stages of a tumor or cancer. The composition can selectively home to one or more particular stages of one or more particular types of tumor. The composition can selectively home to the vasculature of one or more different stages of one or more particular types of tumor.

The cargo molecule can selectively home to a tumor. The cargo molecule can selectively home to tumor vasculature. The cargo molecule can selectively home to one or more particular types of tumor. The cargo molecule can selectively home to the vasculature of one or more particular types of tumor. The cargo molecule can selectively home to one or more particular stages of a tumor or cancer. The cargo molecule can selectively home to the vasculature of one or more particular stages of a tumor or cancer. The cargo molecule can selectively home to one or more particular stages of one or more particular types of tumor. The cargo molecule can selectively home to the vasculature of one or more different stages of one or more particular types of tumor.

The surface molecule can selectively home to a tumor. The surface molecule can selectively home to tumor vasculature. The surface molecule can selectively home to one or more particular types of tumor. The surface molecule can selectively home to the vasculature of one or more particular types of tumor. The surface molecule can selectively home to one or more particular stages of a tumor or cancer. The surface molecule can selectively home to the vasculature of one or more particular stages of a tumor or cancer. The surface molecule can selectively home to one or more particular stages of one or more particular types of tumor. The surface molecule can selectively home to the vasculature of one or more different stages of one or more particular types of tumor.

The membrane perturbing molecule can selectively home to a tumor. The membrane perturbing molecule can selectively home to tumor vasculature. The membrane perturbing molecule can selectively home to one or more particular types of tumor. The membrane perturbing molecule can selectively home to the vasculature of one or more particular types of tumor. The membrane perturbing molecule can selectively home to one or more particular stages of a tumor or cancer. The membrane perturbing molecule can selectively home to the vasculature of one or more particular stages of a tumor or cancer. The membrane perturbing molecule can selectively home to one or more particular stages of one or more particular types of tumor. The membrane perturbing molecule can selectively home to the vasculature of one or more different stages of one or more particular types of tumor.

The disclosed compositions, surface molecules, amino acid sequences, cargo molecules, proteins or peptides can, for example, home to brain cells, brain stem cells, brain tissue, and/or brain vasculature, kidney cells, kidney stem cells, kidney tissue, and/or kidney vasculature, skin cells, skin stem cells, skin tissue, and/or skin vasculature, lung cells, lung tissue, and/or lung vasculature, pancreatic cells, pancreatic tissue, and/or pancreatic vasculature, intestinal cells, intestinal tissue, and/or intestinal vasculature, adrenal gland cells, adrenal tissue, and/or adrenal vasculature, retinal cells, retinal tissue, and/or retinal vasculature, liver cells, liver tissue, and/or liver vasculature, prostate cells, prostate tissue, and/or prostate vasculature, endometriosis cells, endometriosis tissue, and/or endometriosis vasculature, ovary cells, ovary tissue, and/or ovary vasculature, tumor cells, tumors, tumor blood vessels, and/or tumor vasculature, bone cells, bone tissue, and/or bone vasculature, bone marrow cells, bone marrow tissue, and/or bone marrow vasculature, cartilage cells, cartilage tissue, and/or cartilage vasculature, stem cells, embryonic stem cells, pluripotent stem cells, induced pluripotent stem cells, adult stem cells, hematopoietic stem cells, neural stem cells, mesenchymal stem cells, mammary stem cells, endothelial stem cells, olfactory adult stem cells, neural crest stem cells, cancer stem cells, blood cells, erythrocytes, platelets, leukocytes, granulocytes, neutrophils, eosinphils, basophils, lymphoid cells, lymphocytes, monocytes, wound vasculature, vasculature of injured tissue, vasculature of inflamed tissue, atherosclerotic plaques, or a combination.

Examples of homing molecules and homing peptides are known. Examples include: Brain homing peptides such as: CNSRLHLRC (SEQ ID NO:8), CENWWGDVC (SEQ ID NO:9), WRCVLREGPAGGCAWFNRHRL (SEQ ID NO:10), CLSSRLDAC (SEQ ID NO:11), CVLRGGRC (SEQ ID NO:12), CNSRLQLRC (SEQ ID NO:13), CGVRLGC (SEQ ID NO:14), CKDWGRIC (SEQ ID NO:15), CLDWGRIC (SEQ ID NO:16), CTRITESC (SEQ ID NO:17), CETLPAC (SEQ ID NO:18), CRTGTLFC (SEQ ID NO:19), CGRSLDAC (SEQ ID NO:20), CRHWFDVVC (SEQ ID NO:21), CANAQSHC (SEQ ID NO:22), CGNPSYRC (SEQ ID NO:23), YPCGGEAVAGVSSVRTMCSE (SEQ ID NO:24), LNCDYQGTNPATSVSVPCTV (SEQ ID NO:25); kidney homing peptides such as: CLPVASC (SEQ ID NO:26), CGAREMC (SEQ ID NO:27), CKGRSSAC (SEQ ID NO:28), CWARAQGC (SEQ ID NO:29), CLGRSSVC (SEQ ID NO:30), CTSPGGSC (SEQ ID NO:31), CMGRWRLC (SEQ ID NO:32), CVGECGGC (SEQ ID NO:33), CVAWLNC (SEQ ID NO:34), CRRFQDC (SEQ ID NO:35), CLMGVHC (SEQ ID NO:36), CKLLSGVC (SEQ ID NO:37), CFVGHDLC (SEQ ID NO:38), CRCLNVC (SEQ ID NO:39), CKLMGEC (SEQ ID NO:40); skin homing peptides such as: CARSKNKDC (SEQ ID NO:41), CRKDKC (SEQ ID NO:42), CVALCREACGEGC (SEQ ID NO:43), CSSGCSKNCLEMC (SEQ ID NO:44), CIGEVEVC (SEQ ID NO:45), CKWSRLHSC (SEQ ID NO:46), CWRGDRKIC (SEQ ID NO:47), CERVVGSSC (SEQ ID NO:48), CLAKENVVC (SEQ ID NO:49); lung homing peptides such as: CGFECVRQCPERC (SEQ ID NO:50), CGFELETC (SEQ ID NO:51), CTLRDRNC (SEQ ID NO:52), CIGEVEVC (SEQ ID NO:53), CTLRDRNC (SEQ ID NO:54), CGKRYRNC (SEQ ID NO:55), CLRPYLNC (SEQ ID NO:56), CTVNEAYKTRMC (SEQ ID NO:57), CRLRSYGTLSLC (SEQ ID NO:58), CRPWHNQAHTEC (SEQ ID NO:59); pancreas homing peptides such as: SWCEPGWCR (SEQ ID NO:60), CKAAKNK (SEQ ID NO:61), CKGAKAR (SEQ ID NO:62), VGVGEWSV (SEQ ID NO:63); intestine homing peptides such as: YSGKWGW (SEQ ID NO:64); uterus homing peptides such as: GLSGGRS (SEQ ID NO:65); adrenal gland homing peptides such as: LMLPRAD (SEQ ID NO:66), LPRYLLS (SEQ ID NO:67); retina homing peptides such as: CSCFRDVCC (SEQ ID NO:68), CRDVVSVIC (SEQ ID NO:69); gut homing peptides such as: YSGKWGK (SEQ ID NO:70), GISALVLS (SEQ ID NO:71), SRRQPLS (SEQ ID NO:72), MSPQLAT (SEQ ID NO:73), MRRDEQR (SEQ ID NO:74), QVRRVPE (SEQ ID NO:75), VRRGSPQ (SEQ ID NO:76), GGRGSWE (SEQ ID NO:77), FRVRGSP (SEQ ID NO:78), RVRGPER (SEQ ID NO:79); liver homing peptides such as: VKSVCRT (SEQ ID NO:80), WRQNMPL (SEQ ID NO:81), SRRFVGG (SEQ ID NO:82), ALERRSL (SEQ ID NO:83), ARRGWTL (SEQ ID NO:84); prostate homing peptides such as: SMSIARL (SEQ ID NO:85), VSFLEYR (SEQ ID NO:86), RGRWLAL (SEQ ID NO:87); ovary homing peptides such as: EVRSRLS (SEQ ID NO:88), VRARLMS (SEQ ID NO:89), RVGLVAR (SEQ ID NO:90), RVRLVNL (SEQ ID NO:91); Clot binding/homing peptide such as: CREKA (SEQ ID NO:92), CLOT1, and CLOT2; heart homing peptides such as: CRPPR (SEQ ID NO:93), CGRKSKTVC (SEQ ID NO:94), CARPAR (SEQ ID NO:95), CPKRPR (SEQ ID NO:96), CKRAVR (SEQ ID NO:97), CRNSWKPNC (SEQ ID NO:98), RGSSS (SEQ ID NO:99), CRSTRANPC (SEQ ID NO:100), CPKTRRVPC (SEQ ID NO:101), CSGMARTKC (SEQ ID NO:102), GGGVFWQ (SEQ ID NO:103), HGRVRPH (SEQ ID NO:104), VVLVTSS (SEQ ID NO:105), CLHRGNSC (SEQ ID NO:106), CRSWNKADNRSC (SEQ ID NO:107), CGRKSKTVC (SEQ ID NO:108), CKRAVR (SEQ ID NO:109), CRNSWKPNC (SEQ ID NO:110), CPKTRRVPC (SEQ ID NO:111), CSGMARTKC (SEQ ID NO:112), CARPAR (SEQ ID NO:113), CPKRPR (SEQ ID NO:114); tumor blood vessel homing peptide such as: CNGRC (SEQ ID NO:115) and other peptides with the NGR motif (U.S. Pat. Nos. 6,177,542 and 6,576,239; U.S. Patent Application Publication No. 20090257951); RGD peptides, and RGR peptides. Other homing peptides include CSRPRRSEC (SEQ ID NO:116), CSRPRRSVC (SEQ ID NO:117), and CSRPRRSWC (SEQ ID NO:118) (Hoffman et al., Cancer Cell, vol. 4 (2003)), F3 (KDEPQRRSARLSAKPAPPKPEPKPKKAPAKK; SEQ ID NO:119), PQRRSARLSA (SEQ ID NO:120), and PKRRSARLSA (SEQ ID NO:121) (U.S. Pat. No. 7,544,767).

Homing molecules can also be defined by their targets. For example, numerous antigens and proteins are known that can be useful for targeting. Any molecule that can bind, selectively bind, home, selectively, target, selectively target, etc. such target molecules can be used as a homing molecule. For example, antibodies, nucleic acid aptamers, and compounds that can bind to target molecules can be used as homing molecules. Examples of useful target molecules for homing molecules include αv integrins, αvβ3 integrin, αvβ5 integrin, α5β1 integrin, aminopeptidase N, tumor endothelial markers (TEMs), endosialin, p32, gClq receptor, annexin-1, nucleolin, fibronectin ED-B, fibrin-fibronectin complexes, interleukin-11 receptor α, and protease-cleaved collagen IV. These and other examples are described and referred to in Ruoslahti et al., J. Cell Biology, 2010 (doi: 10.1083/jbc.200910104), which is hereby incorporated by reference in its entirety and specifically for its description of and references to target molecules.

The composition can comprise any number of homing molecules. By way of example, the composition can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more homing molecules. The composition can also comprise any number in between those numbers listed above.

Homing molecules can be associated with and arranged in the compositions in a variety of configurations. In some forms, homing molecules can be associated with, conjugated to, and/or covalently coupled to a plurality of surface molecules. In some forms, homing molecules can be associated with, conjugated to, and/or covalently coupled to a plurality of cargo molecules. In some forms, homing molecules can be associated with, conjugated to, and/or covalently coupled to a plurality of cargo molecules, wherein the cargo molecules can be associated with, conjugated to, and/or covalently coupled to a plurality of surface molecules. Combinations of these combinations can also be used.

1. Tumor-Homing Compounds

The disclosed homing molecules can be tumor-homing compounds. Tumor-homing compounds are compounds that selectively home to tumors and tumor-associated tissue. Many compounds that target, bind to, and/or home to tumors are known, most of which can be used as tumor-homing compounds in the disclosed compositions. Tumor-homing compounds can each be independently selected from any known tumor-homing compounds.

Tumor-homing compounds can comprise the amino acid sequence CGKRK (SEQ ID NO:1) or a conservative derivative thereof, the amino acid sequence CRKDKC (SEQ ID NO:2) or a conservative derivative thereof, or a combination. Tumor-homing compounds can comprise the amino acid sequence CGKRK (SEQ ID NO:1) or a conservative variant thereof. In some forms, one or more of the homing molecules can comprise the amino acid sequence CGKRK (SEQ ID NO:1).

Useful peptides for tumor targeting include, for example, the tumor-homing CendR peptide iRGD, LyP-1, a peptide that contains a putative CendR element and has tumor-penetrating properties, and RGR peptides. The LyP-1 peptide has a unique target within tumors; it preferentially accumulates in the hypoxic/low nutrient areas of tumors (Laakkonen et al., 2002; 2004; Karmali et al., 2009). CRGRRST ((SEQ ID NO:122; RGR; Joyce et al., 2003) is a peptide that has been successfully used in targeting a cytokine antibody combination into tumors (Hamzah et al., 2008). This peptide is linear, which simplifies the synthesis. Like LyP-1, RGR is at least to some extent tumor type-specific (Joyce et al., 2003), but the tumor types recognized by the two peptides seem to be partially different, which may be an advantage in testing combinations with the pan-tumor iRGD.

Because tumors can include clot-related proteins, some clot-binding and clot-homing compounds can also be tumor-homing compounds. Such tumor-homing clot-binding compounds can be used as tumor-homing compounds as described herein. Tumor-homing compounds can each be independently selected from, for example, an amino acid segment comprising the amino acid sequence REK, an amino acid segment comprising the amino acid sequence CAR (such as CARSKNKDC (SEQ ID NO:123)), an amino acid segment comprising the amino acid sequence CRK (such as CRKDKC (SEQ ID NO:124)), a fibrin-binding peptide, a peptide that binds clots and not fibrin (such as CGLIIQKNEC(CLT1, SEQ ID NO:125) and CNAGESSKNC(CLT2, SEQ ID NO:126)), a clot-binding antibody, and a clot-binding small organic molecule. A plurality of the clot-binding compounds can each independently comprise an amino acid segment comprising the amino acid sequence REK. Such peptides are also described in U.S. Patent Application Publication No. 2008/0305101, which is hereby incorporated by reference for its description of such peptides. Peptides comprising amino acid sequences CAR or CRK are also described in U.S. Patent Application Publication No. 2009/0036349, which is hereby incorporated by reference for its description of such peptides.

LyP-1 are homing molecules that selectively home to tumor lymphatic vasculature, for example, the lymphatic vasculature of breast cancer tumors and osteosarcomas, in preference to normal lymphatic vasculature. LyP-1 can selectively home, for example, to the lymphatic vasculature of squamous carcinomas. The core LyP-1 peptide has an amino acid sequence CGNKRTRGC (SEQ ID NO:127). LyP-1 peptides are described in U.S. Patent Application Nos. 2004-0087499, 2007-0219134, and 2008-0014143, which are hereby incorporated by reference in their entirety, an specifically for their description of such peptides.

The clot-binding compound can also comprise a fibrin-binding peptide (FBP). Examples of fibrin-binding peptides are known in the art (Van Rooijen N, Sanders A (1994) J Immunol Methods 174: 83-93; Moghimi S M, Hunter A C, Murray J C (2001) Pharmacol Rev 53: 283-318; U.S. Pat. No. 5,792,742, all herein incorporated by reference in their entirety for their teaching concerning fibrin binding peptides).

Clot-binding peptides can also bind to proteins other than fibrin. Example include peptides that bind to fibronectin that has become incorporated into a clot (Pilch et al., (2006) PNAS, 103: 2800-2804, hereby incorporated in its entirety for its teaching concerning clot-binding peptides). Examples of clot-binding peptides include, but is not limited to, CGLIIQKNEC(CLT1, SEQ ID NO:125) and CNAGESSKNC(CLT2, SEQ ID NO:126). The amino acid segments can also be independently selected from amino acid segments comprising the amino acid sequence CLT 1 or CLT2 or a conservative variant thereof, amino acid segments comprising the amino acid sequence CLT1 or CLT2, or amino acid segments consisting of the amino acid sequence CLT1 or CLT2. The amino acid segments can each independently comprise the amino acid sequence CLT1 or CLT2 or a conservative variant thereof. The amino acid segments can also each independently comprise the amino acid sequence CLT1 or CLT2. The amino acid segment can also consist of the amino acid sequence CLT1 or CLT2.

The amino acid segments can also each independently comprise the amino acid sequence CARSKNKDC (SEQ ID NO:128)), and the amino acid sequence CRK (such as CRKDKC (SEQ ID NO:129). Peptides comprising amino acid sequences CAR or CRK are also described in U.S. Patent Application Publication No. 2009/0036349, which is hereby incorporated by reference for its description of such peptides.

The composition can comprise any number of tumor-homing compounds. By way of example, the composition can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more tumor-homing compounds. The composition can also comprise any number in between those numbers listed above.

Table 1 shows examples of tumor-homing CendR peptides.

TABLE 1
Examples of Tumor-Homing
Peptides with CendR Elements

| Sequence | Reference |
|---|---|
| 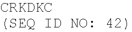 | Jarvinen et al., Am. J. Pathol. 171(2): 702-711 (2007) |
| 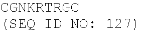 | Laakkonen et al., Nature Medicine 8: 751-755 (2002) |
| 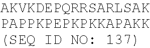 | Christian et al., JCB, 163(4): 871-878 (2003); U.S. Pat. No. 7,544,767 |
| 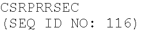 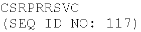 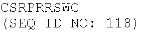 | Hoffman et al., Cancer Cell, vol. 4 (2003) |
| 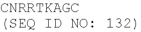 | Zhang et al., Cancer Res. 66(11): 5696-5706 (2006) |
| 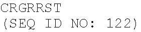 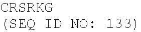 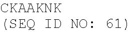 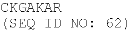 | Joyce et al., 4(5): 393-403 (2003) |
| 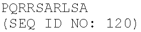 | Porkka et al., Proc. Natl. Acad. Sci. USA 99(11): 7444-7449 (2002); U.S. Pat. No. 7,544,767 |
| 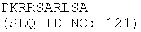 | U.S. Pat. No. 7,544,767 |
| 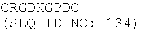 | iRGD, Sugahara et al., Cancer Cell (2009); Sugahara et al. Science (2010); U.S. patent application No. 12/355,672, filed January 19, 2009 |

Tumor-homing compounds can also be modified. Any of the modifications described herein for homing molecules can be used with the disclosed tumor-homing compounds.

2. Modified Homing Molecules

The disclosed homing molecules can include modified forms of homing molecules. The homing molecules can have any useful modification. For example, some modifications can stabilize the homing molecule. For example, the disclosed homing molecules include methylated homing molecules. Methylated homing molecules are particularly useful when the homing molecule includes a protein, peptide or amino acid segment. For example, a homing molecule can be a modified homing molecule, where, for example, the modified homing molecule includes a modified amino acid segment or amino acid sequence. For example, a modified homing molecule can be a methylated homing molecule, where, for example, the methylated homing molecule includes a methylated amino acid segment or amino acid sequence. Other modifications can be used, either alone or in combination. Where the homing molecule is, or includes, a protein, peptide, amino acid segment and/or amino acid sequences, the modification can be to the protein, peptide, amino acid segment, amino acid sequences and/or any amino acids in the protein, peptide, amino acid segment and/or amino acid sequences. Amino acid and peptide modifications are known to those of skill in the art, some of which are described below and elsewhere herein. Methylation is a particularly useful modification for the disclosed homing molecules. Using modified forms of homing molecules can increase the effectiveness of the homing and targeting, which can increase the effect on the target.

A plurality of modified and/or unmodified homing molecules can each be independently selected from, for example, an amino acid segment comprising a modified or unmodified form of the amino acid sequence of a homing peptide, an amino acid segment comprising a modified or unmodified form of the amino acid sequence CGKRK (SEQ ID NO:1), and an amino acid segment comprising a modified or unmodified form of the amino acid sequence CRKDKC (SEQ ID NO:2). A plurality of the homing molecules can each independently comprise an amino acid segment comprising a modified or unmodified form of the amino acid sequence of a homing peptide.

The composition can comprise any number of modified and/or unmodified homing molecules. By way of example, the composition can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more modified and/or unmodified homing molecules. The composition can also comprise any number in between those numbers listed above.

As used herein, a “methylated derivative” of a protein, peptide, amino acid segment, amino acid sequence, etc. refers to a form of the protein, peptide, amino acid segment, amino acid sequence, etc. that is methylated. Unless the context indicates otherwise, reference to a methylated derivative of a protein, peptide, amino acid segment, amino acid sequence, etc. does no include any modification to the base protein, peptide, amino acid segment, amino acid sequence, etc. other than methylation. Methylated derivatives can also have other modifications, but such modifications generally will be noted. For example, conservative variants of an amino acid sequence would include conservative amino acid substitutions of the base amino acid sequence. Thus, reference to, for example, a “methylated derivative” of a specific amino acid sequence “and conservative variants thereof” would include methylated forms of the specific amino acid sequence and methylated forms of the conservative variants of the specific amino acid sequence, but not any other modifications of derivations. As another example, reference to a methylated derivative of an amino acid segment that includes amino acid substitutions would include methylated forms of the amino acid sequence of the amino acid segment and methylated forms of the amino acid sequence of the amino acid segment include amino acid substitutions.

### B. Cargo Molecules

The disclosed compositions include one or more cargo molecules. Generally, the disclosed compositions can include a plurality of cargo molecules. The disclosed compositions can include a single type of cargo molecule or a plurality of different types of cargo molecules. Thus, for example, the disclosed compositions can include a plurality of different types of cargo molecules where a plurality of one or more of the different types of cargo molecules can be present.

Cargo molecules can be any compound, molecule, conjugate, composition, etc. that is desired to be delivered using the disclosed compositions. For example, the cargo molecules can be therapeutic agents, detectable agents, or a combination. For example, the cargo molecules can be membrane perturbing molecules, pro-apoptotic molecules, pore-generating molecules, antimicrobial molecules, mitochondria-affecting molecules, mitochondria-targeted molecules, or a combination. Examples of some useful cargo molecules are described below and elsewhere herein.

Cargo molecules can be associated with and arranged in the compositions in a variety of configurations. In some forms, cargo molecules can be associated with, conjugated to, and/or covalently coupled to a plurality of surface molecules. In some forms, cargo molecules can be associated with, conjugated to, and/or covalently coupled to a plurality of homing molecules. In some forms, cargo molecules can be associated with, conjugated to, and/or covalently coupled to a plurality of homing molecules, wherein the homing molecules can be associated with, conjugated to, and/or covalently coupled to a plurality of surface molecules. Combinations of these combinations can also be used.

1. Membrane Perturbing Molecules

Useful forms of cargo molecules include membrane perturbing molecules. Membrane perturbing molecules include molecules that can disrupt membranes, that can form pores in membranes, that can make membranes leaky, that can be targeted to or affect intracellular membranes or organelles, such mitochondria or lysosomes. Some forms of membrane perturbing molecules can be pro-apoptotic while others can be non-apoptotic. Some forms of membrane perturbing molecules can be pro-apoptotic for only some types of cells.

In some forms, one or more of the homing molecules can comprise the amino acid sequence CGKRK (SEQ ID NO:1). In some forms, one or more of the membrane perturbing molecules can comprise the amino acid sequence _{D}(KLAKLAK)₂ (SEQ ID NO:3) or a conservative variant thereof, (KLAKLAK)₂ (SEQ ID NO:3) or a conservative variant thereof, (KLAKKLA)₂ (SEQ ID NO:5) or a conservative variant thereof, (KAAKKAA)₂ (SEQ ID NO:6) or a conservative variant thereof, (KLGKKLG)₃ (SEQ ID NO:7) or a conservative variant thereof, or a combination. In some forms, one or more of the membrane perturbing molecules can comprise the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), (KLGKKLG)₃ (SEQ ID NO:7), or a combination. In some forms, one or more of the membrane perturbing molecules can comprise the amino acid sequence _{D}(KLAKLAK)₂ (SEQ ID NO:3) or a conservative variant thereof. In some forms, one or more of the membrane perturbing molecules can comprise the amino acid sequence _{D}(KLAKLAK)₂ (SEQ ID NO:3). Membrane perturbing peptides of this type are described in Ellerby, Nature Medicine 5, 1032-1038 (1999), which is hereby incorporated by reference for its description of such peptides.

A plurality of modified and/or unmodified membrane perturbing molecules can each be independently selected from, for example, an amino acid segment comprising a modified or unmodified form of the amino acid sequence of a homing peptide, an amino acid segment comprising a modified or unmodified form of the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), (KLGKKLG)₃ (SEQ ID NO:7), or a combination. A plurality of the membrane perturbing molecules can each independently comprise an amino acid segment comprising a modified or unmodified form of the amino acid sequence of a homing peptide.

The composition can comprise a sufficient number and composition of membrane perturbing molecules (modified or not) such that the composition has a membrane perturbing effect on the target. In one example, sufficiency of the number and composition of modified and/or unmodified membrane perturbing molecules can be determined by assessing membrane disruption, apoptosis, and/or therapeutic effect on the target.

The composition can comprise any number of modified and/or unmodified membrane perturbing molecules. By way of example, the composition can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more modified and/or unmodified membrane perturbing molecules. The composition can also comprise any number in between those numbers listed above.

Membrane perturbing molecules can be associated with and arranged in the compositions in a variety of configurations. In some forms, membrane perturbing molecules can be associated with, conjugated to, and/or covalently coupled to a plurality of surface molecules. In some forms, membrane perturbing molecules can be associated with, conjugated to, and/or covalently coupled to a plurality of homing molecules. In some forms, membrane perturbing molecules can be associated with, conjugated to, and/or covalently coupled to a plurality of homing molecules, wherein the homing molecules can be associated with, conjugated to, and/or covalently coupled to a plurality of surface molecules. Combinations of these combinations can also be used.

i. Modified Membrane Perturbing Molecules

The disclosed membrane perturbing molecules can include modified forms of membrane perturbing molecules. The membrane perturbing molecules can have any useful modification. For example, some modifications can stabilize the membrane perturbing molecule. For example, the disclosed membrane perturbing molecules include methylated membrane perturbing molecules. Methylated membrane perturbing molecules are particularly useful when the membrane perturbing molecule includes a protein, peptide or amino acid segment. For example, a membrane perturbing molecule can be a modified membrane perturbing molecule, where, for example, the modified membrane perturbing molecule includes a modified amino acid segment or amino acid sequence. For example, a modified membrane perturbing molecule can be a methylated membrane perturbing molecule, where, for example, the methylated membrane perturbing molecule includes a methylated amino acid segment or amino acid sequence. Other modifications can be used, either alone or in combination. Where the membrane perturbing molecule is, or includes, a protein, peptide, amino acid segment and/or amino acid sequences, the modification can be to the protein, peptide, amino acid segment, amino acid sequences and/or any amino acids in the protein, peptide, amino acid segment and/or amino acid sequences. Amino acid and peptide modifications are known to those of skill in the art, some of which are described below and elsewhere herein. Methylation is a particularly useful modification for the disclosed membrane perturbing molecules. Using modified forms of membrane perturbing molecules can increase their effectiveness.

2. Moieties

The disclosed compositions can further comprise one or more moieties. The cargo molecules of the disclosed compositions can include one or more moieties. For example, the moieties can be independently selected from the group consisting of an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, an image contrast agent, a fluorophore, fluorescein, rhodamine, a radionuclide, indium-111, technetium-99, carbon-11, and carbon-13. In some forms, at least one of the moieties can be a therapeutic agent. Examples of therapeutic agents are paclitaxel and taxol. In some forms, at least one of the moieties can be a detectable agent. As used herein, the term “moiety” is used broadly to mean a physical, chemical, or biological material that generally imparts a biologically useful function to a linked or conjugated molecule. As disclosed herein, the properties of the moiety can also be found in a surface molecule, or both the surface molecule and the moiety can share one of the traits disclosed herein. For example, the surface molecule can comprise a detectable agent, while the moiety can comprise a therapeutic agent. This also applies for the homing molecules, which can also comprise one or more of the properties of moieties as disclosed herein. The description of therapeutic and detectable agents herein is intended to apply to any of the disclosed cargo molecules, membrane perturbing molecules, moieties, surface molecules, or homing molecules. Thus, for example, moieties can be conjugated to, coupled to, or can be part of, for example, the disclosed surface molecules, homing molecules, membrane perturbing molecules, or compositions comprising, or conjugates of, surface molecules, homing molecules, and membrane perturbing molecules.

A moiety can be any natural or non-natural material including, without limitation, a biological material, such as a cell, phage or other virus; an organic chemical such as a small molecule; a radionuclide; a nucleic acid molecule or oligonucleotide; a polypeptide; or a peptide. Useful moieties include, but are not limited to, therapeutic agents such as cancer chemotherapeutic agents, cytotoxic agents, pro-apoptotic agents, and anti-angiogenic agents; detectable labels and imaging agents; and tags or other insoluble supports. Useful moieties further include, without limitation, phage and other viruses, cells, liposomes, polymeric matrices, non-polymeric matrices or particles such as gold particles, microdevices and nanodevices, and nano-scale semiconductor materials. These and other moieties known in the art can be components of a composition.

In some forms, the moiety can be an RGD peptide, such as iRGD. iRGD peptides and their use are described in U.S. Patent Application Publication 2009-0246133, which is hereby incorporated by reference in its entirety, and specifically for its description of the form, structure, and use of iRGD.

i. Therapeutic Agents

The moiety can be a therapeutic agent. As used herein, the term “therapeutic agent” means a molecule which can have one or more biological activities in a normal or pathologic tissue. A variety of therapeutic agents can be used as a moiety. The therapeutic agent can comprise a compound or composition for treating cancer. The therapeutic agent can comprise a compound or composition to induce programmed cell death or apoptosis. Membrane perturbing molecules are a form of therapeutic agent.

In some embodiments, the therapeutic agent can be a cancer chemotherapeutic agent. As used herein, a “cancer chemotherapeutic agent” is a chemical agent that inhibits the proliferation, growth, life-span or metastatic activity of cancer cells. Such a cancer chemotherapeutic agent can be, without limitation, a taxane such as docetaxel; an anthracyclin such as doxorubicin; an alkylating agent; a vinca alkaloid; an anti-metabolite; a platinum agent such as cisplatin or carboplatin; a steroid such as methotrexate; an antibiotic such as adriamycin; a isofamide; or a selective estrogen receptor modulator; an antibody such as trastuzumab.

Taxanes are chemotherapeutic agents useful with the compositions disclosed herein. Useful taxanes include, without limitation, docetaxel (Taxotere; Aventis Pharmaceuticals, Inc.; Parsippany, N.J.) and paclitaxel (Taxol; Bristol-Myers Squibb; Princeton, N.J.). See, for example, Chan et al., J. Clin. Oncol. 17:2341-2354 (1999), and Paridaens et al., J. Clin. Oncol. 18:724 (2000).

A cancer chemotherapeutic agent useful with the compositions disclosed herein also can be an anthracyclin such as doxorubicin, idarubicin or daunorubicin. Doxorubicin is a commonly used cancer chemotherapeutic agent and can be useful, for example, for treating breast cancer (Stewart and Ratain, In: “Cancer: Principles and practice of oncology” 5th ed., chap. 19 (eds. DeVita, Jr., et al.; J. P. Lippincott 1997); Harris et al., In “Cancer: Principles and practice of oncology,” supra, 1997). In addition, doxorubicin has anti-angiogenic activity (Folkman, Nature Biotechnology 15:510 (1997); Steiner, In “Angiogenesis: Key principles-Science, technology and medicine,” pp. 449-454 (eds. Steiner et al.; Birkhauser Verlag, 1992)), which can contribute to its effectiveness in treating cancer.

An alkylating agent such as melphalan or chlorambucil also can be a useful cancer chemotherapeutic agent. Similarly, a vinca alkaloid such as vindesine, vinblastine or vinorelbine; or an antimetabolite such as 5-fluorouracil, 5-fluorouridine or a derivative thereof can be a useful cancer chemotherapeutic agent.

A platinum agent also can be a useful cancer chemotherapeutic agent. Such a platinum agent can be, for example, cisplatin or carboplatin as described, for example, in Crown, Seminars in Oncol. 28:28-37 (2001). Other useful cancer chemotherapeutic agents include, without limitation, methotrexate, mitomycin-C, adriamycin, ifosfamide and ansamycins.

A cancer chemotherapeutic agent useful for treatment of breast cancer and other hormonally-dependent cancers also can be an agent that antagonizes the effect of estrogen, such as a selective estrogen receptor modulator or an anti-estrogen. The selective estrogen receptor modulator, tamoxifen, is a cancer chemotherapeutic agent that can be used in a composition for treatment of breast cancer (Fisher et al., J. Natl. Cancer Instit. 90:1371-1388 (1998)).

The therapeutic agent can be an antibody such as a humanized monoclonal antibody. As an example, the anti-epidermal growth factor receptor 2 (HER2) antibody, trastuzumab (Herceptin; Genentech, South San Francisco, Calif.) can be a therapeutic agent useful for treating HER2/neu overexpressing breast cancers (White et al., Annu Rev. Med. 52:125-141 (2001)).

Useful therapeutic agents also can be a cytotoxic agent, which, as used herein, can be any molecule that directly or indirectly promotes cell death. Useful cytotoxic agents include, without limitation, small molecules, polypeptides, peptides, peptidomimetics, nucleic acid-molecules, cells and viruses. As non-limiting examples, useful cytotoxic agents include cytotoxic small molecules such as doxorubicin, docetaxel or trastuzumab; antimicrobial peptides such as those described further below; pro-apoptotic polypeptides such as caspases and toxins, for example, caspase-8; diphtheria toxin A chain, Pseudomonas exotoxin A, cholera toxin, ligand fusion toxins such as DAB389EGF, ricinus communis toxin (ricin); and cytotoxic cells such as cytotoxic T cells. See, for example, Martin et al., Cancer Res. 60:3218-3224 (2000); Kreitman and Pastan, Blood 90:252-259 (1997); Allam et al., Cancer Res. 57:2615-2618 (1997); and Osborne and Coronado-Heinsohn, Cancer J. Sci. Am. 2:175 (1996). One skilled in the art understands that these and additional cytotoxic agents described herein or known in the art can be useful in the disclosed compositions and methods.

In one embodiment, a therapeutic agent can be a therapeutic polypeptide. As used herein, a therapeutic polypeptide can be any polypeptide with a biologically useful function. Useful therapeutic polypeptides encompass, without limitation, cytokines, antibodies, cytotoxic polypeptides; pro-apoptotic polypeptides; and anti-angiogenic polypeptides. As non-limiting examples, useful therapeutic polypeptides can be a cytokine such as tumor necrosis factor-α (TNF-α), tumor necrosis factor-β (TNF-(β), granulocyte macrophage colony stimulating factor (GM-CSF), granulocyte colony stimulating factor (G-CSF), interferon-α (IFN-α); interferon-γ (IFN-γ), interleukin-1 (IL-1), interleukin-2 (IL-2), interleukin-3 (IL-3), interleukin-4 (IL-4), interleukin-6 (IL-6), interleukin-7 (IL-7), interleukin-10 (IL-10), interleukin-12 (IL-12), lymphotactin (LTN) or dendritic cell chemokine 1 (DC-CK1); an anti-HER2 antibody or fragment thereof; a cytotoxic polypeptide including a toxin or caspase, for example, diphtheria toxin A chain, Pseudomonas exotoxin A, cholera toxin, a ligand fusion toxin such as DAB389EGF or ricin; or an anti-angiogenic polypeptide such as angiostatin, endostatin, thrombospondin, platelet factor 4; anastellin; or one of those described further herein or known in the art (see below). It is understood that these and other polypeptides with biological activity can be a “therapeutic polypeptide.”

A therapeutic agent can also be an anti-angiogenic agent. As used herein, the term “anti-angiogenic agent” means a molecule that reduces or prevents angiogenesis, which is the growth and development of blood vessels. A variety of anti-angiogenic agents can be prepared by routine methods. Such anti-angiogenic agents include, without limitation, small molecules; proteins such as dominant negative forms of angiogenic factors, transcription factors and antibodies; peptides; and nucleic acid molecules including ribozymes, antisense oligonucleotides, and nucleic acid molecules encoding, for example, dominant negative forms of angiogenic factors and receptors, transcription factors, and antibodies and antigen-binding fragments thereof. See, for example, Hagedorn and Bikfalvi, Crit. Rev. Oncol. Hematol. 34:89-110 (2000), and Kirsch et al., J. Neurooncol. 50:149-163 (2000).

Vascular endothelial growth factor (VEGF) has been shown to be important for angiogenesis in many types of cancer, including breast cancer angiogenesis in vivo (Borgstrom et al., Anticancer Res. 19:4213-4214 (1999)). The biological effects of VEGF include stimulation of endothelial cell proliferation, survival, migration and tube formation, and regulation of vascular permeability. An anti-angiogenic agent can be, for example, an inhibitor or neutralizing antibody that reduces the expression or signaling of VEGF or another angiogenic factor, for example, an anti-VEGF neutralizing monoclonal antibody (Borgstrom et al., supra, 1999). An anti-angiogenic agent also can inhibit another angiogenic factor such as a member of the fibroblast growth factor family such as FGF-1 (acidic), FGF-2 (basic), FGF-4 or FGF-5 (Slavin et al., Cell Biol. Int. 19:431-444 (1995); Folkman and Shing, J. Biol. Chem. 267:10931-10934 (1992)) or an angiogenic factor such as angiopoietin-1, a factor that signals through the endothelial cell-specific Tie2 receptor tyrosine kinase (Davis et al., Cell 87:1161-1169 (1996); and Suri et al., Cell 87:1171-1180 (1996)), or the receptor of one of these angiogenic factors. It is understood that a variety of mechanisms can act to inhibit activity of an angiogenic factor including, without limitation, direct inhibition of receptor binding, indirect inhibition by reducing secretion of the angiogenic factor into the extracellular space, or inhibition of expression, function or signaling of the angiogenic factor.

A variety of other molecules also can function as anti-angiogenic agents including, without limitation, angiostatin; a kringle peptide of angiostatin; endostatin; anastellin, heparin-binding fragments of fibronectin; modified forms of antithrombin; collagenase inhibitors; basement membrane turnover inhibitors; angiostatic steroids; platelet factor 4 and fragments and peptides thereof; thrombospondin and fragments and peptides thereof; and doxorubicin (O'Reilly et al., Cell 79:315-328 (1994)); O'Reilly et al., Cell 88:277-285 (1997); Homandberg et al., Am. J. Path. 120:327-332 (1985); Homandberg et-al., Biochim. Biophys. Acta 874:61-71 (1986); and O'Reilly et al., Science 285:1926-1928 (1999)). Commercially available anti-angiogenic agents include, for example, angiostatin, endostatin, metastatin and 2ME2 (EntreMed; Rockville, Md.); anti-VEGF antibodies such as Avastin (Genentech; South San Francisco, Calif.); and VEGFR-2 inhibitors such as SU5416, a small molecule inhibitor of VEGFR-2 (SUGEN; South San Francisco, Calif.) and SU6668 (SUGEN), a small molecule inhibitor of VEGFR-2, platelet derived growth factor and fibroblast growth factor I receptor. It is understood that these and other anti-angiogenic agents can be prepared by routine methods and are encompassed by the term “anti-angiogenic agent” as used herein.

The compositions disclosed herein can also be used at a site of inflammation or injury. Moieties useful for this purpose can include therapeutic agents belonging to several basic groups including anti-inflammatory agents which prevent inflammation, restenosis preventing drugs which prevent tissue growth, anti-thrombogenic drugs which inhibit or control formation of thrombus or thrombolytics, and bioactive agents which regulate tissue growth and enhance healing of the tissue. Examples of useful therapeutic agents include but are not limited to steroids, fibronectin, anti-clotting drugs, anti-platelet function drugs, drugs which prevent smooth muscle cell growth on inner surface wall of vessel, heparin, heparin fragments, aspirin, coumadin, tissue plasminogen activator (TPA), urokinase, hirudin, streptokinase, antiproliferatives (methotrexate, cisplatin, fluorouracil, Adriamycin), antioxidants (ascorbic acid, beta carotene, vitamin E), antimetabolites, thromboxane inhibitors, non-steroidal and steroidal anti-inflammatory drugs, beta and calcium channel blockers, genetic materials including DNA and RNA fragments, complete expression genes, antibodies, lymphokines, growth factors, prostaglandins, leukotrienes, laminin, elastin, collagen, and integrins.

Useful therapeutic agents also can be antimicrobial peptides. Thus, for example, also disclosed are moieties comprising an antimicrobial peptide, where the composition is selectively internalized and exhibits a high toxicity to the targeted area. Useful antimicrobial peptides can have low mammalian cell toxicity when not incorporated into the composition. As used herein, the term “antimicrobial peptide” means a naturally occurring or synthetic peptide having antimicrobial activity, which is the ability to kill or slow the growth of one or more microbes. An antimicrobial peptide can, for example, kill or slow the growth of one or more strains of bacteria including a Gram-positive or Gram-negative bacteria, or a fungi or protozoa. Thus, an antimicrobial peptide can have, for example, bacteriostatic or bacteriocidal activity against, for example, one or more strains of Escherichia coli, Pseudomonas aeruginosa or Staphylococcus aureus. While not wishing to be bound by the following, an antimicrobial peptide can have biological activity due to the ability to form ion channels through membrane bilayers as a consequence of self-aggregation.

An antimicrobial peptide is typically highly basic and can have a linear or cyclic structure. As discussed further below, an antimicrobial peptide can have an amphipathic .alpha.-helical structure (see U.S. Pat. No. 5,789,542; Javadpour et al., J. Med. Chem. 39:3107-3113 (1996); and Blondelle and Houghten, Biochem. 31: 12688-12694 (1992)). An antimicrobial peptide also can be, for example, a β-strand/sheet-forming peptide as described in Mancheno et al., J. Peptide Res. 51:142-148 (1998).

An antimicrobial peptide can be a naturally occurring or synthetic peptide. Naturally occurring antimicrobial peptides have been isolated from biological sources such as bacteria, insects, amphibians, and mammals and are thought to represent inducible defense proteins that can protect the host organism from bacterial infection. Naturally occurring antimicrobial peptides include the gramicidins, magainins, mellitins, defensins and cecropins (see, for example, Maloy and Kari, Biopolymers 37:105-122 (1995); Alvarez-Bravo et al., Biochem. J. 302:535-538 (1994); Bessalle et al., FEBS 274:-151-155 (1990.); and Blondelle and Houghten in Bristol (Ed.), Annual Reports in Medicinal Chemistry pages 159-168 Academic Press, San Diego). An antimicrobial peptide also can be an analog of a natural peptide, especially one that retains or enhances amphipathicity (see below).

An antimicrobial peptide incorporated into the composition disclosed herein can have low mammalian cell toxicity when linked to the composition. Mammalian cell toxicity readily can be assessed using routine assays. As an example, mammalian cell toxicity can be assayed by lysis of human erythrocytes in vitro as described in Javadpour et al., supra, 1996. An antimicrobial peptide having low mammalian cell toxicity is not lytic to human erythrocytes or requires concentrations of greater than 100 μM for lytic activity, preferably concentrations greater than 200, 300, 500 or 1000 μM.

In one embodiment, disclosed are compositions in which the antimicrobial peptide portion promotes disruption of mitochondrial membranes when internalized by eukaryotic cells. In particular, such an antimicrobial peptide preferentially disrupts mitochondrial membranes as compared to eukaryotic membranes. Mitochondrial membranes, like bacterial membranes but in contrast to eukaryotic plasma membranes, have a high content of negatively charged phospholipids. An antimicrobial peptide can be assayed for activity in disrupting mitochondrial membranes using, for example, an assay for mitochondrial swelling or another assay well known in the art.

An antimicrobial peptide that induces significant mitochondrial swelling at, for example, 50 μM, 40 μM, 30 μM, 20 μM, 10 μM, or less, is considered a peptide that promotes disruption of mitochondrial membranes.

Antimicrobial peptides generally have random coil conformations in dilute aqueous solutions, yet high levels of helicity can be induced by helix-promoting solvents and amphipathic media such as micelles, synthetic bilayers or cell membranes. α-Helical structures are well known in the art, with an ideal α-helix characterized by having 3.6 residues per turn and a translation of 1.5 Å per residue (5.4 Å per turn; see Creighton, Proteins: Structures and Molecular Properties W. H Freeman, New York (1984)). In an amphipathic a-helical structure, polar and non-polar amino acid residues are aligned into an amphipathic helix, which is a a-helix in which the hydrophobic amino acid residues are predominantly on one face, with hydrophilic residues predominantly on the opposite face when the peptide is viewed along the helical axis.

Antimicrobial peptides of widely varying sequence have been isolated, sharing an amphipathic α-helical structure as a common feature (Saberwal et al., Biochim. Biophys. Acta 1197:109-131 (1994)). Analogs of native peptides with amino acid substitutions predicted to enhance amphipathicity and helicity typically have increased antimicrobial activity. In general, analogs with increased antimicrobial activity also have increased cytotoxicity against mammalian cells (Maloy et al., Biopolymers 37:105-122 (1995)).

As used herein in reference to an antimicrobial peptide, the term “amphipathic α-helical structure” means an a-helix with a hydrophilic face containing several polar residues at physiological pH and a hydrophobic face containing nonpolar residues. A polar residue can be, for example, a lysine or arginine residue, while a nonpolar residue can be, for example, a leucine or alanine residue. An antimicrobial peptide having an amphipathic .alpha.-helical structure generally has an equivalent number of polar and nonpolar residues within the amphipathic domain and a sufficient number of basic residues to give the peptide an overall positive charge at neutral pH (Saberwal et al., Biochim. Biophys. Acta 1197:109-131 (1994)). One skilled in the art understands that helix-promoting amino acids such as leucine and alanine can be advantageously included in an antimicrobial peptide (see, for example, Creighton, supra, 1984). Synthetic, antimicrobial peptides having an amphipathic α-helical structure are known in the art, for example, as described in U.S. Pat. No. 5,789,542 to McLaughlin and Becker.

It is understood by one skilled in the art of medicinal oncology that these and other agents are useful therapeutic agents, which can be used separately or together in the disclosed compositions and methods. Thus, it is understood that the compositions disclosed herein can contain one or more of such therapeutic agents and that additional components can be included as part of the composition, if desired. As a non-limiting example, it can be desirable in some cases to utilize an oligopeptide spacer between the surface molecule and the homing molecule and/or cargo molecules (Fitzpatrick and Garnett, Anticancer Drug Des. 10:1-9 (1995)).

Other useful agents include thrombolytics, aspirin, anticoagulants, painkillers and tranquilizers, beta-blockers, ace-inhibitors, nitrates, rhythm-stabilizing drugs, and diuretics. Agents that limit damage to the heart work best if given within a few hours of the heart attack. Thrombolytic agents that break up blood clots and enable oxygen-rich blood to flow through the blocked artery increase the patient's chance of survival if given as soon as possible after the heart attack. Thrombolytics given within a few hours after a heart attack are the most effective. Injected intravenously, these include anisoylated plasminogen streptokinase activator complex (APSAC) or anistreplase, recombinant tissue-type plasminogen activator (r-tPA), and streptokinase. The disclosed compounds can use any of these or similar agents.

Some other examples of useful therapeutic agents include nitrogen mustards, nitrosoureas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors and hormonal agents. Exemplary chemotherapy drugs are Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Chlomaphazine, Cholophosphamide, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Estramustine, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, mechlorethamine oxide hydrochloride, Melphalan, Mercaptopurine, Methotrexate, Mitomycin, Mitotane, Mitoxantrone, Novembiehin, Oxaliplatin, Paclitaxel, Pamidronate, Pentostatin, Phenesterine, Plicamycin, Prednimustine, Procarbazine, Rituximab, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Trofosfamide, Vinblastine, Vincristine, Vindesine, Vinorelbine, VP-16, and Xeloda. Alkylating agents such as Thiotepa and; alkyl sulfonates such as Busulfan, Improsulfan and Piposulfan; aziridines such as Benzodopa, Carboquone, Meturedopa, and Uredopa; ethylenimines and methylamelamines including altretamine, triethylenemelamine, trietylenephosphoramide, triethylenethiophosphaoramide and trimethylolomelamine; nitroureas such as Cannustine, Chlorozotocin, Fotemustine, Lomustine, Nimustine, and Ranimustine; antibiotics such as Aclacinomysins, Actinomycin, Authramycin, Azaserine, Bleomycins, Cactinomycin, Calicheamicin, Carabicin, Caminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites such as Methotrexate and 5-fluorouracil (5-FU); folic acid analogues such as Denopterin, Methotrexate, Pteropterin, and Trimetrexate; purine analogs such as Fludarabine, 6-mercaptopurine, Thiamiprine, and Thioguanine; pyrimidine analogs such as Ancitabine, Azacitidine, 6-azauridine, Carmofur, Cytarabine, Dideoxyuridine, Doxifluridine, Enocitabine, Floxuridine, and 5-FU; androgens such as Calusterone, Dromostanolone Propionate, Epitiostanol, Rnepitiostane, and Testolactone; anti-adrenals such as aminoglutethimide, Mitotane, and Trilostane; folic acid replenisher such as frolinic acid; aceglatone; aldophosphamide glycoside; aminolevulinic acid; Amsacrine; Bestrabucil; Bisantrene; Edatraxate; Defofamine; Demecolcine; Diaziquone; Elfornithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethylhydrazide; Procarbazine; PSK®; Razoxane; Sizofrran; Spirogermanium; tenuazonic acid; triaziquone; 2,2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, e.g., Paclitaxel (TAXOL®, Bristol-Myers Squibb Oncology, Princeton, N.J.) and Doxetaxel (TAXOTERE®, Rhone-Poulenc Rorer, Antony, France); Gemcitabine; 6-thioguanine; Mercaptopurine; Methotrexate; platinum analogs such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylornithine (DMFO); retinoic acid; Esperamicins; Capecitabine; and pharmaceutically acceptable salts, acids or derivatives of any of the above. Also included are anti-hormonal agents that act to regulate or inhibit hormone action on tumors such as anti-estrogens including for example Tamoxifen, Raloxifene, aromatase inhibiting 4(5)-imidazoles, 4 Hydroxytamoxifen, Trioxifene, Keoxifene, Onapristone, And Toremifene (Fareston); and anti-androgens such as Flutamide, Nilutamide, Bicalutamide, Leuprolide, and Goserelin; and pharmaceutically acceptable salts, acids or derivatives of any of the above. Useful cargo molecules include, for example, doxorubicin, Herceptin, and liposomal doxorubicin.

The cargo molecules can also comprise a boron containing compound. Boron containing compounds have received increasing attention as therapeutic agents over the past few years as technology in organic synthesis has expanded to include this atom (Boron Therapeutics on the horizon, Groziak, M. P.; American Journal of Therapeutics (2001) 8, 321-328). The most notable boron containing therapeutic is the boronic acid bortezomib which was recently launched for the treatment of multiple myeloma. This breakthrough demonstrates the feasibility of using boron containing compounds as pharmaceutical agents. Boron containing compounds have been shown to have various biological activities including herbicides (Organic boron compounds as herbicides. Barnsley, G. E.; Eaton, J. K.; Airs, R. S.; (1957), DE 1016978 19571003), boron neutron capture therapy (Molecular Design and Synthesis of B-10 Carriers for Neutron Capture Therapy. Yamamoto, Y.; Pure Appl. Chem., (1991) 63, 423-426), serine protease inhibition (Borinic acid inhibitors as probes of the factors involved in binding at the active sites of subtilisin Carlsberg and alpha-chymotrypsin. Simpelkamp, J.; Jones, J. B.; Bioorganic & Medicinal Chemistry Letters, (1992), 2(11), 1391-4; Design, Synthesis and Biological Evaluation of Selective Boron-containing Thrombin Inhibitors. Weinand, A.; Ehrhardt, C.; Metternich, R.; Tapparelli, C.; Bioorganic and Medicinal Chemistry, (1999), 7, 1295-1307), acetylcholinesterase inhibition (New, specific and reversible bifunctional alkylborinic acid inhibitor of acetylcholinesterase. Koehler, K. A.; Hess, G. P.; Biochemistry (1974), 13, 5345-50) and as antibacterial agents (Boron-Containing Antibacterial Agents Effects on Growth and Morphology of Bacteria Under Various Culture Conditions. Bailey, P. J.; Cousins, G.; Snow, G. A.; and White, A. J.; Antimicrobial Agents and Chemotherapy, (1980), 17, 549-553). The boron containing compounds with antibacterial activity can be sub-divided into two main classes, the diazaborinines, which have been known since the 1960's, and dithienylborinic acid complexes. This latter class has been expanded to include many different diarylborinic acid complexes with potent antibacterial activity (Preparation of diarylborinic acid esters as DNA methyl transferase inhibitors. Benkovic, S. J.; Shapiro, L.; Baker, S. J.; Wahnon, D.C.; Wall, M.; Shier, V. K.; Scott, C. P.; Baboval, J.; PCT Int. Appl. (2002), WO 2002044184).

ii. Detectable Agents

The moiety in the disclosed compositions can also be a detectable agent. A variety of detectable agents are useful in the disclosed methods. As used herein, the term “detectable agent” refers to any molecule which can be detected. Useful detectable agents include compounds and molecules that can be administered in vivo and subsequently detected. Detectable agents useful in the disclosed compositions and methods include yet are not limited to radiolabels and fluorescent molecules. The detectable agent can be, for example, any molecule that facilitates detection, either directly or indirectly, preferably by a non-invasive and/or in vivo visualization technique. For example, a detectable agent can be detectable by any known imaging techniques, including, for example, a radiological technique, a magnetic resonance technique, or an ultrasound technique. Detectable agents can include, for example, a contrasting agent, e.g., where the contrasting agent is ionic or non-ionic. In some embodiments, for instance, the detectable agent comprises a tantalum compound and/or a barium compound, e.g., barium sulfate. In some embodiments, the detectable agent comprises iodine, such as radioactive iodine. In some embodiments, for instance, the detectable agent comprises an organic iodo acid, such as iodo carboxylic acid, triiodophenol, iodoform, and/or tetraiodoethylene. In some embodiments, the detectable agent comprises a non-radioactive detectable agent, e.g., a non-radioactive isotope. For example, Gd can be used as a non-radioactive detectable agent in certain embodiments.

Other examples of detectable agents include molecules which emit or can be caused to emit detectable radiation (e.g., fluorescence excitation, radioactive decay, spin resonance excitation, etc.), molecules which affect local electromagnetic fields (e.g., magnetic, ferromagnetic, ferromagnetic, paramagnetic, and/or superparamagnetic species), molecules which absorb or scatter radiation energy (e.g., chromophores and/or fluorophores), quantum dots, heavy elements and/or compounds thereof. See, e.g., detectable agents described in U.S. Publication No. 2004/0009122. Other examples of detectable agents include a proton-emitting molecules, a radiopaque molecules, and/or a radioactive molecules, such as a radionuclide like Tc-99m and/or Xe-13. Such molecules can be used as a radiopharmaceutical. In still other embodiments, the disclosed compositions can comprise one or more different types of detectable agents, including any combination of the detectable agents disclosed herein.

Useful fluorescent moieties include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Particularly useful fluorescent labels include fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio. Fluorescent probes and there use are also described in Handbook of Fluorescent Probes and Research Products by Richard P. Haugland.

Further examples of radioactive detectable agents include gamma emitters, e.g., the gamma emitters In-111, I-125 and 1-131, Rhenium-186 and 188, and Br-77 (see. e.g., Thakur, M. L. et al., Throm Res. Vol. 9 pg. 345 (1976); Powers et al., Neurology Vol. 32 pg. 938 (1982); and U.S. Pat. No. 5,011,686); positron emitters, such as Cu-64, C-11, and O-15, as well as Co-57, Cu-67, Ga-67, Ga-68, Ru-97, Tc-99m, In-113m, Hg-197, Au-198, and Pb-203. Other radioactive detectable agents can include, for example tritium, C-14 and/or thallium, as well as Rh-105, 1-123, Nd-147, Pm-151, Sm-153, Gd-159, Tb-161, Er-171 and/or T1-201.

The use of Technitium-99m (Tc-99m) is preferable and has been described in other applications, for example, see U.S. Pat. No. 4,418,052 and U.S. Pat. No. 5,024,829. Tc-99m is a gamma emitter with single photon energy of 140 keV and a half-life of about 6 hours, and can readily be obtained from a Mo-99/Tc-99 generator.

In some embodiments, compositions comprising a radioactive detectable agent can be prepared by coupling a targeting moiety with radioisotopes suitable for detection. Coupling can occur via a chelating agent such as diethylenetriaminepentaacetic acid (DTPA), 4,7,10-tetraazacyclododecane-N-,N′,N″,N′″-tetraacetic acid (DOTA) and/or metallothionein, any of which can be covalently attached to the targeting moiety. In some embodiments, an aqueous mixture of technetium-99m, a reducing agent, and a water-soluble ligand can be prepared and then allowed to react with a disclosed targeting moiety. Such methods are known in the art, see e.g., International Publication No. WO 99/64446. In some embodiments, compositions comprising radioactive iodine, can be prepared using an exchange reaction. For example, exchange of hot iodine for cold iodine is well known in the art. Alternatively, a radio-iodine labeled compound can be prepared from the corresponding bromo compound via a tributylstannyl intermediate.

Magnetic detectable agents include paramagnetic contrasting agents, e.g., gadolinium diethylenetriaminepentaacetic acid, e.g., used with magnetic resonance imaging (MRI) (see, e.g., De Roos, A. et al., Int. J. Card. Imaging Vol. 7 pg. 133 (1991)). Some preferred embodiments use as the detectable agent paramagnetic atoms that are divalent or trivalent ions of elements with an atomic number 21, 22, 23, 24, 25, 26, 27, 28, 29, 42, 44, 58, 59, 60, 61, 62, 63, 64, 65, 66, 67, 68, 69, or 70. Suitable ions include, but are not limited to, chromium(III), manganese(II), iron(II), iron(III), cobalt(II), nickel(II), copper(II), praseodymium(III), neodymium(III), samarium(III) and ytterbium(III), as well as gadolinium(III), terbiurn(III), dysoprosium(III), holmium(III), and erbium(III). Some preferred embodiments use atoms with strong magnetic moments, e.g., gadolinium(III).

In some embodiments, compositions comprising magnetic detectable agents can be prepared by coupling a targeting moiety with a paramagnetic atom. For example, the metal oxide or a metal salt, such as a nitrate, chloride or sulfate salt, of a suitable paramagnetic atom can be dissolved or suspended in a water/alcohol medium, such as methyl, ethyl, and/or isopropyl alcohol. The mixture can be added to a solution of an equimolar amount of the targeting moiety in a similar water/alcohol medium and stirred. The mixture can be heated moderately until the reaction is complete or nearly complete. Insoluble compositions formed can be obtained by filtering, while soluble compositions can be obtained by evaporating the solvent. If acid groups on the chelating moieties remain in the disclosed compositions, inorganic bases (e.g., hydroxides, carbonates and/or bicarbonates of sodium, potassium and/or lithium), organic bases, and/or basic amino acids can be used to neutralize acidic groups, e.g., to facilitate isolation or purification of the composition.

The detectable agent can be coupled to the composition in such a way so as not to interfere with the ability of the homing molecule to interact with the target site. In some embodiments, the detectable agent can be chemically bound to, for example, the surface molecule, homing molecule, and/or membrane perturbing molecule. In some embodiments, the detectable agent can be chemically bound to a moiety that is itself chemically bound to, for example, the surface molecule, homing molecule, and/or membrane perturbing molecule, indirectly linking the imaging and targeting moieties.

### C. Internalization Elements and Tissue Penetration Elements

The disclosed compositions, surface molecules, cargo molecules, peptides, proteins, amino acid sequences, etc. can comprise one or more internalization elements, tissue penetration elements, or both. Internalization elements and tissue penetration elements can be incorporated into or fused with other peptide components of the composition, such as peptide homing molecules and peptide cargo molecules. Internalization elements are molecules, often peptides or amino acid sequences, that allow the internalization element and components with which it is associated, to pass through biological membranes. Tissue penetration elements are molecules, often peptides or amino acid sequences, that allow the tissue penetration element and components with which it is associated to passage into and through tissue. “Internalization” refers to passage through a plasma membrane or other biological barrier. “Penetration” refers to passage into and through a membrane, cell, tissue, or other biological barrier. Penetration generally involves and includes internalization. Some molecules, such as CendR elements, function as both internalization elements and tissue penetration elements.

Internalization elements include, for example, cell-penetrating peptides (CPPs) and CendR peptides. Peptides that are internalized into cells are commonly referred to as cell-penetrating peptides. There are two main classes of such peptides: hydrophobic and cationic (Zorko and Langel, 2005). The cationic peptides, which are commonly used to introduce nucleic acids, proteins into cells, include the prototypic cell-penetrating peptides (CPP), Tat, and penetratin (Derossi et al., 1998; Meade and Dowdy, 2007). A herpes virus protein, VP22, is capable of both entering and exiting cells and carrying a payload with it (Elliott and O'Hare, 1997; Brewis et al., 2003).

1. CendR Elements

Useful forms of internalization elements and tissue penetration elements are CendR elements. CendR elements are amino acid sequences with a C-terminal element as a defining feature that signals highly efficient internalization of phage and free peptides into cells. This internalization phenomenon has been named the “C-end rule” or “CendR”. The CendR pathway can also be used for passage of compositions of interest from the vasculature and their spread into tissue. The C-terminal element can cause spread of compositions from the vasculature (and thus can be spread into tumor tissue from an intravenous injection, for example). CendR elements can also be used to mediate passage of compositions of interest through other CendR-capable membranes, such as mucous membranes and the blood-brain barrier. As used herein, “tissue penetration” and “penetration of tissue” refer to passage into or through a tissue beyond or through the outer or a first layer of cells or through a tissue membrane. Such passage or penetration through tissue (which can also be referred to as extravasation and tissue penetration) can be a function of, for example, cell internalization and passage between cells in the tissue. Throughout this application, when the term “tissue penetration” is used, it is understood that such penetration can also extend to other barriers and CendR-capable membranes found throughout the body, such as the blood brain barrier.

Unlike the known cell-penetrating peptides, the CendR internalizing element is position-dependent—it is inactive when present in positions other than the C-terminus of the peptide. Another distinguishing feature is that the CendR element is stereo-specific, that is, CendR elements composed of D-amino acids are inactive. A latent CendR peptide can be activated by cleavage by, for example, the appropriate proteolytic enzyme to expose, for example, a C-terminal arginine, lysine, or lysine-glycine. Throughout the application, when the term “CendR element” or “C-terminal element” is used, it is used to describe a C-terminal arginine, a C-terminal lysine, or a C-terminal lysine-glycine pair, where glycine is at the furthest C-terminal position. In other words, in the case where a lysine is on the C terminus end, the CendR element can remain functional with a glycine on the C terminus side of the lysine. However, it is not necessary to have glycine on the end in order for the lysine residue to be functional as a C-terminal element, so that lysine can be present without glycine and still be functional. The converse is not true, however, in that glycine cannot function as a C-terminal element without the presence of lysine adjacent to it. Arginine does not require either lysine or glycine to function as a C-terminal element, as long as it remains in the furthest C-terminal position. Such CendR elements can be referred to as type 1 CendR elements.

The term “CendR element” or “C-terminal element” can also be used to describe a C-terminal histidine and amino acid sequences having the sequence X₁X₂X₃X₄, where X₁ can be R, K or H, where X₄ can be R, K, H, or KG, and where X₂ and X₃ can each be, independently, any amino acid. Such CendR elements can be referred to as type 2 CendR elements. The X₂ and X₃ amino acids can be selected for specific purposes. For example, X₂, X₃, or both can be chosen to form all or a portion of a protease recognition sequence. This would be useful, for example, to specify or enable cleavage of a peptide having the CendR element as a latent or cryptic CendR element that is activated by cleavage following the X₄ amino acid. Examples of such amino acid choices are shown in Tables 1 and 2. The X₁, X₂ and X₃ amino acids can also be selected, for example, to recruit additional proteins to NRP-1 molecules at the cell surface. This can be applied, for example, to modulate the selectivity and internalization and/or tissue penetration potency of CendR elements (and the compositions, conjugates, proteins, and peptides containing CendR elements). The X₂ and X₃ amino acids can also be selected to prevent protease cleavage within the X₁-X₄ motif. Optionally, certain amino acids can also be excluded from use for X₂, X₃, or both. For example, if desired, G and D can be excluded from simultaneous use as X₂ and X₃, respectively. Some type 2 CendR elements can also be described as R/K/HXXR/K/H (SEQ ID NO:130) and R/K/HXXKG (SEQ ID NO:131).

Examples of CendR elements include XXR/K/H, XXR/K, XXR/H, XXK/H, XXR, XXK, XXH, XXKG, RXXR/K/H, RXXR/K, RXXR/H, RXXK/H, RXXR, RXXK, RXH, RXXKG, KXXR/K/H, KXXR/K, KXXR/H, KXXK/H, KXXR, KXXK, KXXH, KXXKG, HXXR/K/H, HXXR/K, HXXR/H, HXXK/H, HXXR, HXXK, HXXH, HXXKG, R/K/HXXR, R/KXXR, R/HXXR, K/HXXR, RXXR, KXXR, HXXR, R/K/HXXK, R/KXXK, R/HXXK, K/HXXK, RXXK, KXXK, HXXK, R/K/HXXH, R/KXXH, R/HXXH, K/HXXH, RXXH, KXXH, HXXH, R/K/HXXKG, R/KXXKG, R/HXXKG, K/HXXKG, RXXKG, KXXKG, and HXXKG. A CendR element that can be internalized into a cell can be referred to as an internalization CendR element. A CendR element that can penetrate tissue can be referred to as a penetrating CendR element. A CendR element that can be internalized into a cell and that can penetrate tissue can be referred to as an internalization and penetrating CendR element. Unless the context clearly indicates otherwise, reference to “CendR element” refers to any of these, either individually, collectively, or in any combination.

As used herein, “CendR composition” refers to a composition that comprises a CendR element. The CendR element can be, for example, active, activatable, or blocked. For example, the CendR composition can comprise a protein or peptide comprising an amino acid sequence that comprises a CendR element where the amino acid sequence is at the C-terminal end of the protein or peptide.

As used herein, “activatable CendR element” refers to a CendR element having a molecule, moiety, nanoparticle, compound or other composition covalently coupled to the CendR element, such as to the terminal carboxyl group of the C-terminal element, where the molecule, moiety, nanoparticle, compound or other composition can block internalization and/or tissue penetration of the CendR composition, conjugate, molecule, protein, peptide, etc. and where the molecule, moiety, nanoparticle, compound or other composition can be removed (to expose the terminal carboxy group, for example). For example, the activatable CendR element can be on the C-terminal end of the peptide, and can prevent the CendR element from being internalized and/or from penetrating tissue. The molecule, nanoparticle, moiety, compound or other composition covalently coupled to the CendR element can be referred to as the “blocking group.” For example, the blocking group can be coupled to the terminal carboxyl group of the C-terminal arginine or lysine or other C-terminal amino acid of the CendR element, to the C-terminal amino acid of the CendR element, or to an amino acid of the CendR element other than the C-terminal amino acid. The blocking group can also be coupled, or associated with a part of a CendR composition, conjugate, molecule, protein, peptide, etc. other than the CendR element so long as it can prevent the CendR element from being internalized and/or from penetrating tissue. A CendR composition comprising an activatable CendR element can be referred to as an activatable CendR composition. A CendR molecule comprising an activatable CendR element can be referred to as an activatable CendR molecule. A CendR conjugate comprising an activatable CendR element can be referred to as an activatable CendR conjugate. A CendR protein comprising an activatable CendR element can be referred to as an activatable CendR protein. A CendR peptide comprising an activatable CendR element can be referred to as an activatable CendR peptide.

An activatable CendR element can be blocked from internalization into a cell, from tissue penetration, or both. Generally, an activatable CendR element will be blocked from both internalization into a cell and penetration of tissue. Such activatable CendR elements can be referred to as activatable internalization and penetrating CendR elements. However, some activatable CendR elements could be blocked only from tissue penetration or only from internalization into a cell. Such activatable CendR elements can be referred to as activatable internalization CendR elements (for CendR elements that are blocked only from internalization into a cell) or as activatable internalization and penetrating CendR elements (for CendR elements that are blocked only from penetration of tissue). Generally, internalization CendR elements that are activatable will be activatable internalization CendR elements. Similarly, penetrating CendR elements that are activatable generally will be activatable penetrating CendR elements. Internalization and penetrating CendR elements that are activatable will be activatable internalization and penetrating CendR elements. Removal of the blocking group will allow the CendR element to be internalized into a cell, penetrate tissue, or both.

The cleavable bond of an activatable CendR element can be cleaved in any suitable way. For example, the cleavable bond can be cleaved enzymatically or non-enzymatically. For enzymatic cleavage, the cleaving enzyme can be supplied or can be present at a site where the CendR element is delivered, homes, travels or accumulates. For example, the enzyme can be present in proximity to a cell to which the CendR element is delivered, homes, travels, or accumulates. For non-enzymatic cleavage, the CendR element can be brought into contact with a cleaving agent, can be placed in cleaving conditions, or both. A cleaving agent is any substance that can mediate or stimulate cleavage of the cleavable bond. A non-enzymatic cleaving agent is any cleaving agent except enzymes. Cleaving conditions can be any solution or environmental conditions that can mediate or stimulate cleavage of the cleavable bond. For example, some labile bonds can be cleaved in acid conditions, alkaline conditions, in the presence of a reactive group, etc. Non-enzymatic cleaving conditions are any cleaving conditions except the presence of enzymes. Non-agent cleaving conditions are any cleaving conditions except the presence of cleaving agents.

A “protease-activatable CendR element” (or “protease-activated CendR element”) refers to an activatable CendR element where the blocking group is coupled to the CendR element via a peptide bond and where the peptide bond can be cleaved by a protease. Cleavage of this peptide bond in a protease-activatable CendR element makes the CendR element capable of internalization into a cell and/or of tissue penetration. In one example, the blocking group can be coupled to the CendR element via a cleavable or labile bond. The cleavable bond can be cleaved by, for example, an enzyme or a chemical compound. Cleavage or ‘labilization’ bond in an activatable CendR element makes the CendR element capable of internalization into a cell and/or of tissue penetration. Such cleavage or ‘labilization’ can be referred to as activation of the CendR element. A protease-activatable CendR element is a form of activatable CendR element.

Proteolysis that uncovers a C-terminal element can serve as a switch that triggers the internalization signal. Various compositions can be internalized through this mechanism. For example, homing molecule-mediated accumulation can occur at a target site with cell type-specific proteolysis that exposes a C-terminal element which allows for highly specific homing systems with target-triggered internalization. This protease-controllable internalization system can be useful in engineering compositions with functions such as cell type-specific and/or tissue type-specific uptake and the ability to spread the compositions in tissues.

CendR elements are further described in U.S. Patent Application Publication Nos. 2009-0226372 and 2010-0322862, which are hereby incorporated by reference in their entirety, and specifically for their description of the form, structure, and use of CendR elements and peptides.

### D. Surface Molecules

The surface molecules, alternatively referred to as a surface particles, disclosed herein can be conjugated with homing molecules and cargo molecules in such a way that the composition is delivered to a target. The surface molecule can be any substance that can be used with the homing molecules and cargo molecules, and is not restricted by size or substance. Examples include, but are not limited to, nanoparticles (such as iron oxide nanoparticles or albumin nanoparticles), liposomes, small organic molecules, microparticles, or microbubbles, such as fluorocarbon microbubbles. The term surface molecule is used to identify a component of the disclosed composition but is not intended to be limiting. In particular, the disclosed surface molecules are not limited to substances, compounds, compositions, particles or other materials composed of a single molecule. Rather, the disclosed surface molecules are any substance(s), compound(s), composition(s), particle(s) and/or other material(s) that can be conjugated with a plurality of homing molecules and cargo molecules such that at least some of the homing molecules and/or cargo molecules are presented and/or accessible on the surface of the surface molecule. A variety of examples of suitable surface molecules are described and disclosed herein.

The surface molecule can be detectable, or can be a therapeutic agent such as iRGD, RGD, or Abraxane™. The section herein which discusses cargo molecules and moieties that can be detectable or therapeutic also applies to the surface molecule.

Surface molecules can be associated with and arranged in the compositions in a variety of configurations. In some forms, surface molecules can be associated with, conjugated to, and/or covalently coupled to a plurality of homing molecules, a plurality of cargo molecules, or both. In some forms, surface molecules can be associated with, conjugated to, and/or covalently coupled to a plurality of homing molecules, wherein the homing molecules can be associated with, conjugated to, and/or covalently coupled to a plurality of cargo molecules. In some forms, surface molecules can be associated with, conjugated to, and/or covalently coupled to a plurality of cargo molecules, wherein the cargo molecules can be associated with, conjugated to, and/or covalently coupled to a plurality of homing molecules. Combinations of these combinations can also be used.

1. Nanoparticles, Microparticles, and Microbubbles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, nanoworms, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

The nanoparticles can be comprised of cargo molecules and a carrier protein (such as albumin). Such nanoparticles are useful, for example, to deliver hydrophobic or poorly soluble compounds. Nanoparticles of poorly water soluble drugs (such as taxane) have been disclosed in, for example, U.S. Pat. Nos. 5,916,596; 6,506,405; and 6,537,579 and also in U.S. Pat. Pub. No. 2005/0004002A1.

In forms, the nanoparticles can have an average or mean diameter of no greater than about 1000 nanometers (nm), such as no greater than about any of 900, 800, 700, 600, 500, 400, 300, 200, and 100 nm. In some forms, the average or mean diameters of the nanoparticles can be no greater than about 200 nm. In some forms, the average or mean diameters of the nanoparticles can be no greater than about 150 nm. In some forms, the average or mean diameters of the nanoparticles can be no greater than about 100 nm. In some forms, the average or mean diameter of the nanoparticles can be about 20 to about 400 nm. In some forms, the average or mean diameter of the nanoparticles can be about 40 to about 200 nm. In some embodiments, the nanoparticles are sterile-filterable.

The nanoparticles can be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

Examples of suitable carrier proteins include proteins normally found in blood or plasma, which include, but are not limited to, albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the carrier protein is non-blood protein, such as casein, .alpha.-lactalbumin, and .beta.-lactoglobulin. The carrier proteins may either be natural in origin or synthetically prepared. In some embodiments, the pharmaceutically acceptable carrier comprises albumin, such as human serum albumin. Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA 237:355-360, 460-463 (1977)) and Houser et al., Surgery, Gynecology and Obstetrics, 150:811-816 (1980)) and in conjunction with exchange transfusion in the treatment of neonatal hyperbilirubinemia (see, e.g., Finlayson, Seminars in Thrombosis and Hemostasis, 6:85-120 (1980)). Other albumins are contemplated, such as bovine serum albumin. Use of such non-human albumins could be appropriate, for example, in the context of use of these compositions in non-human mammals, such as the veterinary (including domestic pets and agricultural context).

Carrier proteins (such as albumin) in the composition generally serve as a carrier for the hydrophobic cargo molecules, i.e., the carrier protein in the composition makes the cargo molecules more readily suspendable in an aqueous medium or helps maintain the suspension as compared to compositions not comprising a carrier protein. This can avoid the use of toxic solvents (or surfactants) for solubilizing the cargo molecules, and thereby can reduce one or more side effects of administration of the cargo molecules into an individual (such as a human). Thus, in some embodiments, the composition described herein can be substantially free (such as free) of surfactants, such as Cremophor (including Cremophor EL® (BASF)). In some embodiments, the composition can be substantially free (such as free) of surfactants. A composition is “substantially free of Cremophor” or “substantially free of surfactant” if the amount of Cremophor or surfactant in the composition is not sufficient to cause one or more side effect(s) in an individual when the composition is administered to the individual.

The amount of carrier protein in the composition described herein will vary depending on other components in the composition. In some embodiments, the composition comprises a carrier protein in an amount that is sufficient to stabilize the cargo molecules in an aqueous suspension, for example, in the form of a stable colloidal suspension (such as a stable suspension of nanoparticles). In some embodiments, the carrier protein is in an amount that reduces the sedimentation rate of the cargo molecules in an aqueous medium. For particle-containing compositions, the amount of the carrier protein also depends on the size and density of nanoparticles of the cargo molecules.

Methods of making nanoparticle compositions are known in the art. For example, nanoparticles containing cargo molecules and carrier protein (such as albumin) can be prepared under conditions of high shear forces (e.g., sonication, high pressure homogenization, or the like). These methods are disclosed in, for example, U.S. Pat. Nos. 5,916,596; 6,506,405; and 6,537,579 and also in U.S. Pat. Pub. No. 2005/0004002A1.

Briefly, the hydrophobic carrier molecules can be dissolved in an organic solvent, and the solution can be added to a human serum albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride and chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

The nanoparticle can also be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells. Targeting molecules can be attached to the disclosed compositions and/or carriers. For example, the targeting molecules can be antibodies or fragments thereof, ligands for specific receptors, or other proteins specifically binding to the surface of the cells to be targeted.

2. Liposomes

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

3. Micelles

“Micelle” as used herein refers to a structure comprising an outer lipid monolayer. Micelles can be formed in an aqueous medium when the Critical Micelle Concentration (CMC) is exceeded. Small micelles in dilute solution at approximately the critical micelle concentration (CMC) are generally believed to be spherical. However, under other conditions, they may be in the shape of distorted spheres, disks, rods, lamellae, and the like. Micelles formed from relatively low molecular weight amphiphile molecules can have a high CMC so that the formed micelles dissociate rather rapidly upon dilution. If this is undesired, amphiphile molecules with large hydrophobic regions can be used. For example, lipids with a long fatty acid chain or two fatty acid chains, such as phospholipids and sphingolipids, or polymers, specifically block copolymers, can be used.

Polymeric micelles have been prepared that exhibit CMCs as low as 10⁻⁶ M (molar). Thus, they tend to be very stable while at the same time showing the same beneficial characteristics as amphiphile micelles. Any micelle-forming polymer presently known in the art or as such may become known in the future may be used in the disclosed compositions and methods. Examples of micelle-forming polymers include, without limitation, methoxy poly(ethylene glycol)-b-poly(ε-caprolactone), conjugates of poly(ethylene glycol) with phosphatidyl-ethanolamine, poly(ethylene glycol)-b-polyesters, poly(ethylene glycol)-b-poly(L-aminoacids), poly(N-vinylpyrrolidone)-bl-poly(orthoesters), poly(N-vinylpyrrolidone)-b-polyanhydrides and poly(N-vinylpyrrolidone)-b-poly(alkyl acrylates).

Micelles can be produced by processes conventional in the art. Examples of such are described in, for example, Liggins (Liggins, R. T. and Burt, H. M., “Polyether-polyester diblock copolymers for the preparation of paclitaxel loaded polymeric micelle formulations.” Adv. Drug Del. Rev. 54: 191-202, (2002)); Zhang, et al. (Zhang, X. et al., “Development of amphiphilic dibiock copolymers as micellar carriers of taxol.” Int. J. Pharm. 132: 195-206, (1996)); and Churchill (Churchill, J. R., and Hutchinson, F. G., “Biodegradable amphipathic copolymers.” U.S. Pat. No. 4,745,160, (1988)). In one such method, polyether-polyester block copolymers, which are amphipathic polymers having hydrophilic (polyether) and hydrophobic (polyester) segments, are used as micelle forming carriers.

Another type of micelle can be formed using, for example, AB-type block copolymers having both hydrophilic and hydrophobic segments, as described in, for example, Tuzar (Tuzar, Z. and Kratochvil, P., “Block and graft copolymer micelles in solution.”, Adv. Colloid Interface Sci. 6:201-232, (1976)); and Wilhelm, et al. (Wilhelm, M. et al., “Poly(styrene-ethylene oxide) block copolymer micelle formation in water: a fluorescence probe study.”, Macromolecules 24: 1033-1040 (1991)). These polymeric micelles are able to maintain satisfactory aqueous stability. These micelles, in the range of approximately <200 nm in size, are effective in reducing non-selective RES scavenging and show enhanced permeability and retention.

Further, U.S. Pat. No. 5,929,177 to Kataoka, et al. describes a polymeric molecule which is usable as, inter alia, a drug delivery carrier. The micelle is formed from a block copolymer having functional groups on both of its ends and which comprises hydrophilic/hydrophobic segments. The polymer functional groups on the ends of the block copolymer include amino, carboxyl and mercapto groups on the .alpha.-terminal and hydroxyl, carboxyl group, aldehyde group and vinyl group on the .omega.-terminal. The hydrophilic segment comprises polyethylene oxide, while the hydrophobic segment is derived from lactide, lactone or (meth)acrylic acid ester.

Further, for example, poly(D,L-lactide)-b-methoxypolyethylene glycol (MePEG:PDLLA) diblock copolymers can be made using MePEG 1900 and 5000. The reaction can be allowed to proceed for 3 hr at 160° C., using stannous octoate (0.25%) as a catalyst. However, a temperature as low as 130° C. can be used if the reaction is allowed to proceed for about 6 hr, or a temperature as high as 190° C. can be used if the reaction is carried out for only about 2 hr.

As another example, N-isopropylacrylamide (“IPAAm”) (Kohjin, Tokyo, Japan) and dimethylacrylamide (“DMAAm”) (Wako Pure Chemicals, Tokyo, Japan) can be used to make hydroxyl-terminated poly(IPAAm-co-DMAAm) in a radical polymerization process, using the method of Kohori, F. et al. (1998). (Kohori, F. et al., “Preparation and characterization of thermally Responsive block copolymer micelles comprising poly(N-isopropylacrylamide-b-D,L-lactide).” J. Control. Rel. 55: 87-98, (1998)). The obtained copolymer can be dissolved in cold water and filtered through two ultrafiltration membranes with a 10,000 and 20,000 molecular weight cut-off. The polymer solution is first filtered through a 20,000 molecular weight cut-off membrane. Then the filtrate was filtered again through a 10,000 molecular weight cut-off membrane. Three molecular weight fractions can be obtained as a result, a low molecular weight, a middle molecular weight, and a high molecular weight fraction. A block copolymer can then be synthesized by a ring opening polymerization of D,L-lactide from the terminal hydroxyl group of the poly(IPAAm-co-DMAAm) of the middle molecular weight fraction. The resulting poly(IPAAm-co-DMAAm)-b-poly(D,L-lactide) copolymer can be purified as described in Kohori, F. et al. (1999). (Kohori, F. et al., “Control of adriamycin cytotoxic activity using thermally responsive polymeric micelles composed of poly(N-isopropylacrylamide-co-N,N-dimethylacrylamide)-b-poly(D,L-lacide).-”, Colloids Surfaces B: Biointerfaces 16: 195-205, (1999)).

Examples of block copolymers from which micelles can be prepared which can be used to coat a support surface are found in U.S. Pat. No. 5,925,720, to Kataoka, et al., U.S. Pat. No. 5,412,072 to Sakarai, et al., U.S. Pat. No. 5,410,016 to Kataoka, et al., U.S. Pat. No. 5,929,177 to Kataoka, et al., U.S. Pat. No. 5,693,751 to Sakurai, et al., U.S. Pat. No. 5,449,513 to Yokoyama, et al., WO 96/32434, WO 96/33233 and WO 97/0623, the contents of all of which are incorporated by reference. Modifications thereof which are prepared by introducing thereon a suitable functional group (including an ethyleneically unsaturated polymerizable group) are also examples of block copolymers from which micelles of the present invention are preferably prepared. Preferable block copolymers are those disclosed in the above-mentioned patents and or international patent publications. If the block copolymer has a sugar residue on one end of the hydrophilic polymer segment, as in the block copolymer of WO 96/32434, the sugar residue should preferably be subjected to Malaprade oxidation so that a corresponding aldehyde group may be formed.

4. Lipids

Lipids are synthetically or naturally-occurring molecules which includes fats, waxes, sterols, prenol lipids, fat-soluble vitamins (such as vitamins A, D, E and K), glycerolipids, monoglycerides, diglycerides, triglycerides, glycerophospholipids, sphingolipids, phospholipids, fatty acids monoglycerides, saccharolipids and others. Lipids can be hydrophobic or amphiphilic small molecules; the amphiphilic nature of some lipids allows them to form structures such as monolayers, vesicles, micelles, liposomes, bi-layers or membranes in an appropriate environment i.e. aqueous environment. Any of a number of lipids can be used as amphiphile molecules, including amphipathic, neutral, cationic, and anionic lipids. Such lipids can be used alone or in combination, and can also include bilayer stabilizing components such as polyamide oligomers (see, e.g., U.S. Pat. No. 6,320,017, “Polyamide Oligomers”, by Ansell), peptides, proteins, detergents, lipid-derivatives, such as PEG coupled to phosphatidylethanolamine and PEG conjugated to ceramides (see, U.S. Pat. No. 5,885,613). In a preferred embodiment, cloaking agents, which reduce elimination of liposomes by the host immune system, can also be included, such as polyamide-oligomer conjugates, e.g., ATTA-lipids, (see, U.S. patent application Ser. No. 08/996,783, filed Feb. 2, 1998) and PEG-lipid conjugates (see, U.S. Pat. Nos. 5,820,873, 5,534,499 and 5,885,613).

Any of a number of neutral lipids can be included, referring to any of a number of lipid species which exist either in an uncharged or neutral zwitterionic form at physiological pH, including diacylphosphatidylcholine, diacylphosphatidylethanolamine, ceramide, sphingomyelin, cephalin, cholesterol, cerebrosides, and diacylglycerols.

Cationic lipids, carry a net positive charge at physiological pH, can readily be used as amphiphile molecules. Such lipids include, but are not limited to, N,N-dioleyl-N,N-dimethylammonium chloride (“DODAC”); N-(2,3-dioleyloxy) propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3.beta.—(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as LIPOFECTIN (including DOTMA and DOPE, available from GIBCO/BRL), LIPOFECTAMINE (comprising DOSPA and DOPE, available from GIBCO/BRL), and TRANSFECTAM (comprising DOGS, in ethanol, from Promega Corp.).

Anionic lipids can be used as amphiphile molecules and include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Amphiphatic lipids can also be suitable amphiphile molecules. “Amphipathic lipids” refer to any suitable material, wherein the hydrophobic portion of the lipid material orients into a hydrophobic phase, while the hydrophilic portion orients toward the aqueous phase. Such compounds include, but are not limited to, fatty acids, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols. Zwitterionic lipids are a form of amphiphatic lipid.

Sphingolipids are fatty acids conjugated to the aliphatic amino alcohol sphingosine. The fatty acid can be covalently bond to sphingosine via an amide bond. Any amino acid as described above can be covalently bond to sphingosine to form a sphingolipid. A sphingolipid can be further modified by covalent bonding through the α-hydroxyl group. The modification can include alkyl groups, alkenyl groups, alkynyl groups, aromatic groups, heteroaromatic groups, cyclyl groups, heterocyclyl groups, phosphonic acid groups. Non-limiting examples of shingolipids are N-acylsphingosine, N-Acylsphingomyelin, Forssman antigen.

Saccharolipids are compounds that contain both fatty acids and sugars. The fatty acids are covalently bonded to a sugar backbone. The sugar backbone can contain one or more sugars. The fatty acids can bond to the sugars via either amide or ester bonds. The sugar can be any sugar base. The fatty acid can be any fatty acid as described elsewhere herein. The provided compositions can comprise either natural or synthetic saccharolipids. Non-limiting saccharolipids are UDP-3-O-(β-hydroxymyristoyl)-GlcNAc, lipid IV A, Kdo2-lipid A.

### E. Linkers

Disclosed are linkers for associating components of the disclosed compositions. Such linkers can be any molecule, conjugate, composition, etc. that can be used to associate components of the disclosed compositions. Generally, linkers can be used to associate components other than surface molecules to surface molecules. Useful linkers include materials that are biocompatible, have low bioactivity, have low antigenicity, etc. That is, such useful linker materials can serve the linking/association function without adding unwanted bioreactivity to the disclosed compositions. Many such materials are know and used for similar linking and association functions. Polymer materials are a particularly useful form of linker material. For example, polyethylene glycols can be used.

Linkers are useful for achieving useful numbers and densities of the components (such as homing molecules and membrane perturbing molecules) on surface molecules. For example, linkers of fibrous form are useful for increasing the number of components per surface molecule or per a given area of the surface molecule. Similarly, linkers having a branching form are useful for increasing the number of components per surface molecule or per a given area of the surface molecule. Linkers can also have a branching fibrous form.

Linkers of different lengths can be used to bind the disclosed components to surface molecules and to each other. A flexible linker can function well even if relatively short, while a stiffer linker may can be longer to allow effective exposure and density. The length of a linker can refer to the number of atoms in a continuous covalent chain between the attachment points on the components being linked or to the length (in nanometers, for example) of a continuous covalent chain between the attachment points on the components being linked. Unless the context clearly indicates otherwise, the length refers to the shortest continuous covalent chain between the attachment points on the components being linked not accounting for side chains, branches, or loops. Due to flexibility of the linker, all of the linkers may not have same distance from the surface molecule. Thus linkers with different chain lengths can make the resulting composition more effective (by increasing density, for example). Branched linkers bearing multiple components also allow attachment of more than one component at a given site of the surface molecule. Useful lengths for linkers include at least, up to, about, exactly, or between 10, 15, 20, 25, 30, 35, 40, 45, 50, 60, 70, 80, 90, 100, 120, 140, 150, 160, 180, 200, 250, 300, 350, 400, 450, 500, 550, 600, 650, 700, 750, 800, 850, 900, 950, 1,000, 2,000, 3,000, 4,000, 5,000, 6,000, 7,000, 8,000, 9,000, and 10,000 atoms. Useful lengths for linkers include at least, up to, about, exactly, or between 10, 15, 20, 25, 30, 35, 40, 45, 50, 60, 70, 80, 90, 100, 120, 140, 150, 160, 180, 200, 250, 300, 350, 400, 450, 500, 550, 600, 650, 700, 750, 800, 850, 900, 950, 1,000, 2,000, 3,000, 4,000, 5,000, 6,000, 7,000, 8,000, 9,000, and 10,000 nanometers. Any range of these lengths and all lengths between the listed lengths are specifically contemplated.

Hydrophilic or water-solubility linkers can increase the mobility of the attached components. Examples of water-soluble, biocompatible polymers which can serve as linkers include, but are not limited to polymers such polyethylene glycol (PEG), polyethylene oxide (PEO), polyvinyl alcohol, polyhydroxyethyl methacrylate, polyacrylamide, and natural polymers such as hyaluronic acid, chondroitin sulfate, carboxymethylcellulose, and starch. Useful forms of branched tethers include star PEO and comb PEO. Star PEO can be formed of many PEO “arms” emanating from a common core.

Polyethylene glycols (PEGs) are simple, neutral polyethers which have been given much attention in biotechnical and biomedical applications (Milton Harris, J. (ed) “Poly(ethylene glycol) chemistry, biotechnical and biomedical applications” Plenum Press, New York, 1992). PEGs are soluble in most solvents, including water, and are highly hydrated in aqueous environments, with two or three water molecules bound to each ethylene glycol segment; this hydration phenomenon has the effect of preventing adsorption either of other polymers or of proteins onto PEG-modified surfaces. Furthermore, PEGs may readily be modified and bound to other molecules with only little effect on their chemistry. Their advantageous solubility and biological properties are apparent from the many possible uses of PEGs and copolymers thereof, including block copolymers such as PEG-polyurethanes and PEG-polypropylenes. Appropriate molecular weights for PEG linkers used in the disclosed compositions can be from about 120 daltons to about 20 kilodaltons. For example, PEGs can be at least, up to, about, exactly, or between 100, 150, 200, 250, 300, 350, 400, 450, 500, 600, 700, 800, 900, 1000, 1200, 1400, 1500, 1600, 1800, 2000, 2500, 3000, 3500, 4000, 4500, 5000, 5500, 6000, 6500, 7000, 7500, 8000, 8500, 9000, 9500, 10,000, 20,000, 30,000, 40,000, and 50,000 daltons. Any range of these masses and all masses between the listed masses are specifically contemplated. PEGs are usually available as mixtures of somewhat heterogeneous masses with a stated average mass (PEG-5000, for example).

The disclosed compositions can be produced using any suitable techniques. Many techniques, reactive groups, chemistries, etc. for linking components of the types disclosed herein are known and can be used with the disclosed components and compositions. Examples of some techniques for producing the disclosed compositions are described in the examples.

Protein crosslinkers that can be used to crosslink other molecules, elements, moieties, etc. to the disclosed compositions, surface molecules, homing molecules, membrane perturbing molecules, internalization elements, tissue penetration elements, cargo compositions, CendR elements, compositions, proteins, peptides, amino acid sequences, etc. are known in the art and are defined based on utility and structure and include DSS (Disuccinimidylsuberate), DSP (Dithiobis(succinimidylpropionate)), DTSSP (3,3′-Dithiobis (sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl) aminobenzoate), SULFO SIAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP(N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent; Methyl-p-hydroxybenzimidate hydrochloride, 98%), DMA (Dimethyladipimidate hydrochloride).

Components of the disclosed compositions, such as surface molecules, homing molecules, membrane perturbing molecules, internalization elements, tissue penetration elements, etc., can also be coupled using, for example, maleimide coupling. By way of illustration, components can be coupled to lipids by coupling to, for example, 1,2-distearoyl-sn-glycero-3-phosphoethanolamine-N-[maleimide(polyethylene glycol)₂₀₀₀; DSPE-PEG₂₀₀₀-maleimide] (Avanti Polar Lipids) by making use of a free cysteine sulfhydryl group on the component. The reaction can be performed, for example, in aqueous solution at room temperature for 4 hours. This coupling chemistry can be used to couple components of co-compositions and cargo compositions.

Components of the disclosed compositions, such as surface molecules, homing molecules, membrane perturbing molecules, internalization elements, tissue penetration elements, etc., can also be coupled using, for example, amino group-functionalized dextran chemistry. Particles, such as, for example, nanoparticles, nanoworms, and micelles, can be coated with amino group functionalized dextran. Attachment of PEG to aminated particles increases the circulation time, presumably by reducing the binding of plasma proteins involved in opsonization (Moghimi et al., Pharm. Rev. 53, 283-318 (2001)). The particles can have surface modifications, for example, for reticuloendothelial system avoidance (PEG) and homing (homing molecules), endosome escape (pH-sensitive peptide; for example, Pirollo et al., Cancer Res.67, 2938-43 (2007)), a detectable agent, a therapeutic compound, or a combination. To accommodate all these functions on one particle, optimization studies can be conducted to determine what proportion of the available linking sites at the surface of the particles any one of these elements should occupy to give the best combination of targeting and payload delivery. The cell internalization and/or tissue penetration of such compositions can be mediated by the disclosed CendR elements, amino acid sequences, peptides, proteins, molecules, conjugates, and compositions.

The provided peptides and polypeptides can have additional N-terminal, C-terminal, or intermediate amino acid sequences, e.g., amino acid linkers or tags. The term “amino acid linker” refers to an amino acid sequences or insertions that can be used to connect or separate two distinct peptides, polypeptides, or polypeptide fragments, where the linker does not otherwise contribute to the essential function of the composition. The term “amino acid tag” refers to a distinct amino acid sequence that can be used to detect or purify the provided polypeptide, wherein the tag does not otherwise contribute to the essential function of the composition. The provided peptides and polypeptides can further have deleted N-terminal, C-terminal or intermediate amino acids that do not contribute to the essential activity of the peptides and polypeptides.

Components can be directly or indirectly covalently bound to surface molecules or each other by any functional group (e.g., amine, carbonyl, carboxyl, aldehyde, alcohol). For example, one or more amine, alcohol or thiol groups on the components can be reacted directly with isothiocyanate, acyl azide, N-hydroxysuccinimide ester, aldehyde, epoxide, anhydride, lactone, or other functional groups incorporated onto the surface molecules or other components. Schiff bases formed between the amine groups on the components and aldehyde groups on the surface molecule or other components can be reduced with agents such as sodium cyanoborohydride to form hydrolytically stable amine links (Ferreira et al., J. Molecular Catalysis B: Enzymatic 2003, 21, 189-199). Components can be coupled to surface molecules and other components by, for example, the use of a heterobifunctional silane linker reagent, or by other reactions that activate functional groups on either the surface molecule or the components.

Useful modes for linking components to surface molecules and to tother components include heterobifunctional linkers or spacers. Such linkers can have both terminal amine and thiol reactive functional groups for reacting amines on components with sulfhydryl groups, thereby coupling the components in an oriented way. These linkers can contain a variable number of atoms. Examples of such linkers include, but are not limited to, N-Succinimidyl 3-(2-pyridyldithio)propionate (SPDP, 3- and 7-atom spacer), long-chain-SPDP (12-atom spacer), (Succinimidyloxycarbonyl-a-methyl-2-(2-pyridyldithio) toluene) (SMPT, 8-atom spacer), Succinimidyl-4-(N-maleimidomethyl)cyclohexane-1-carboxylate) (SMCC, 11-atom spacer) and Sulfosuccinimidyl-4-(N-maleimidomethyl)cyclohexane-1-carboxylate, (sulfo-SMCC, 11-atom spacer), m-Maleimidobenzoyl-N hydroxysuccinimide ester (MBS, 9-atom spacer), N-(g-maleimidobutyryloxy)succinimide ester (GMBS, 8-atom spacer), N-(g-maleimidobutyryloxy) sulfosuccinimide ester (sulfo-GMBS, 8-atom spacer), Succinimidyl 6-((iodoacetyl) amino) hexanoate (SIAX, 9-atom spacer), Succinimidyl 6-(6-(((4-iodoacetyl)amino)hexanoyl)amino)hexanoate (SIAXX, 16-atom spacer), and p-nitrophenyl iodoacetate (NPIA, 2-atom spacer). One ordinarily skilled in the art also will recognize that a number of other coupling agents or links, with different number of atoms, may be used.

Hydrophilic spacer atoms can be incorporated into linkers to increase the distance between the reactive functional groups. For example, polyethylene glycol (PEG) can be incorporated into sulfo-GMBS. Hydrophilic molecules such as PEG have also been shown to decrease non-specific binding (NSB) and increase hydrophilicity of surfaces when covalently coupled. PEG can also be used as the primary linker material.

Free amine groups of components can also be attached to surface molecules or other components containing reactive amine groups via homobifunctional linkers. Linkers such as dithiobis(succinimidylpropionate) (DSP, 8-atom spacer), disuccinimidyl suberate (DSS, 8-atom spacer), glutaraldehyde (4-atom spacer), Bis[2-(succinimidyloxycarbonyloxy)ethyl]sulfone (BSOCOES, 9-atom spacer), all requiring high pH, can be used for this purpose. Examples of homobifunctional sulfhydryl-reactive linkers include, but are not limited to, 1,4-Di-[3″-2″-pyridyldithio)propion-amido]butane (DPDPB, 16-atom spacer) and Bismaleimidohexane (BMH, 14-atom spacer). For example, these homobifunctional linkers are first reacted with a thiolated surface in aqueous solution (for example PBS, pH 7.4), and then in a second step, the thiolated antibody or protein is joined by the link. Homo- and heteromultifunctional linkers can also be used.

Direct binding of components to thiol, amine, or carboxylic acid functional groups on surface molecules and other components be used to produce compositions which exhibit viral binding (due to increased density of components, for example), resulting in enhanced sensitivity.

As an example, when necessary to achieve high peptide coupling density, additional amino groups can be added to the surface molecules (such as commercially obtained SPIO) as follows: First, to crosslink the particles before the amination step, 3 ml of the colloid (˜10 mgFe/ml in double-distilled water) was added to 5 ml of 5M NaOH and 2 ml of epichlorohydrin (Sigma, St. Louis, Mo.). The mixture was agitated for 24 hours at room temperature to promote interaction between the organic phase (epichlorohydrin) and aqueous phase (dextran-coated particle colloid). In order to remove excess epichlorohydrin, the reacted mixture was dialyzed against double-distilled water for 24 hours using a dialysis cassette (10,000 Da cutoff, Pierce, Rockford Ill.). Amino groups were added to the surface of the particles as follows: 0.02 ml of concentrated ammonium hydroxide (30%) was added to 1 ml of colloid (˜10 mg Fe/ml). The mixture was agitated at room temperature for 24 hours. The reacted mixture was dialyzed against double-distilled water for 24 hours. To further rinse the particles, the colloid was trapped on a MACS®Midi magnetic separation column (Miltenyi Biotec, Auburn Calif.), rinsed with PBS three times, and eluted from the column with 1 ml PBS.

To conjugate CGKRK peptide (and other peptides) to SPIO, the particles were re-suspended at a concentration of 1 mg Fe/ml, and heterobifunctional linker N-[a-maleimidoacetoxy]succinimide ester (AMAS; Pierce) was added (2.5 mg linker per 2 mg Fe) under vortexing. After incubation at room temperature for 40 min, the particles were washed 3 times with 10 ml PBS on a MACS column. The peptide with free terminal cysteine was then added (100 μg peptide per 2 mg Fe). After incubation overnight at 4° C. the particles were washed again and re-suspended in PBS at a concentration of 0.35 mg/ml of Fe). To quantify the number of peptide molecules conjugated to the particles, a known amount of stock or AMAS-activated particles was incubated with varying amounts of the peptide. After completion of the incubation the particles were pelleted at 100.000G using Beckman TLA 100.3 ultracentrifuge rotor (30 min) and the amount of the unbound peptide was quantified by fluorescence. To cleave the conjugated peptide from the particles, the particles were incubated at 37° C. overnight at pH 10. The concentration of free peptide in the supernatant was determined by reading fluorescence and by using the calibration curve obtained for the same peptide. The fluorescence intensity of known amounts of particles was plotted as a function of peptide conjugation density, and the slope equation was used to determine conjugation density in different batches.

### F. Peptides and Amino Acid Segments

In some forms, the homing molecule, cargo molecule, internalization element, tissue penetration element, etc. can be or include a peptide, peptidomimetic, and/or amino acid segment. Unless the context indicates otherwise, reference herein to “peptide” is intended to refer also to amino acid segments, which can form a part of, or constitute an entire, peptide. The disclosed peptides can be in isolated form. As used herein in reference to the disclosed peptides, the term “isolated” means a peptide that is in a form that is relatively free from material such as contaminating polypeptides, lipids, nucleic acids and other cellular material that normally is associated with the peptide in a cell or that is associated with the peptide in a library or in a crude preparation.

The disclosed peptides and amino acid segments can have any suitable length. The disclosed peptides can have, for example, a relatively short length of less than six, seven, eight, nine, ten, 12, 15, 20, 25, 30, 35 or 40 residues. The disclosed peptides also can be useful in the context of a significantly longer sequence. Thus, the peptides can have, for example, a length of up to 50, 100, 150, 200, 250, 300, 400, 500, 1000 or 2000 residues. In particular embodiments, a peptide can have a length of at least 10, 20, 30, 40, 50, 60, 70, 80, 90, 100 or 200 residues. In further embodiments, a peptide can have a length of 5 to 200 residues, 5 to 100 residues, 5 to 90 residues, 5 to 80 residues, 5 to 70 residues, 5 to 60 residues, 5 to 50 residues, 5 to 40 residues, 5 to 30 residues, 5 to 20 residues, 5 to 15 residues, 5 to 10 residues, 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues. As used herein, the term “residue” refers to an amino acid or amino acid analog.

The disclosed amino acid segments can have, for example, a relatively short length of less than six, seven, eight, nine, ten, 12, 15, 20, 25, 30, 35 or 40 residues. The disclosed amino acid segments also can be useful in the context of a significantly longer sequence. Thus, the amino acid segments can have, for example, a length of up to 50, 100, 150, 200, 250, 300, 400, 500, 1000 or 2000 residues. In particular embodiments, an amino acid segment can have a length of at least 10, 20, 30, 40, 50, 60, 70, 80, 90, 100 or 200 residues. In further embodiments, an amino acid segment can have a length of 5 to 200 residues, 5 to 100 residues, 5 to 90 residues, 5 to 80 residues, 5 to 70 residues, 5 to 60 residues, 5 to 50 residues, 5 to 40 residues, 5 to 30 residues, 5 to 20 residues, 5 to 15 residues, 5 to 10 residues, 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues. As used herein, the term “residue” refers to an amino acid or amino acid analog.

As this specification discusses various proteins, protein sequences, peptides, peptides sequences, and amino acid sequences, it is understood that the nucleic acids that can encode those sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. The disclosed peptides and proteins can be coupled to each other via peptide bonds to form fusion peptides and proteins.

The disclosed peptides and amino acid segments can be modified. As used herein, a “methylated derivative” of a protein, peptide, amino acid segment, amino acid sequence, etc. refers to a form of the protein, peptide, amino acid segment, amino acid sequence, etc. that is methylated. Unless the context indicates otherwise, reference to a methylated derivative of a protein, peptide, amino acid segment, amino acid sequence, etc. does no include any modification to the base protein, peptide, amino acid segment, amino acid sequence, etc. other than methylation. Methylated derivatives can also have other modifications, but such modifications generally will be noted. For example, conservative variants of an amino acid sequence would include conservative amino acid substitutions of the based amino acid sequence. Thus, reference to, for example, a “methylated derivative” of a specific amino acid sequence “and conservative variants thereof” would include methylated forms of the specific amino acid sequence and methylated forms of the conservative variants of the specific amino acid sequence, but not any other modifications of derivations. As another example, reference to a methylated derivative of an amino acid segment that includes amino acid substitutions would include methylated forms of the amino acid sequence of the amino acid segment and methylated forms of the amino acid sequence of the amino acid segment include amino acid substitutions.

Protein variants and derivatives are well understood by those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure.

As used herein in reference to a specified amino acid sequence, a “conservative variant” is a sequence in which a first amino acid is replaced by another amino acid or amino acid analog having at least one biochemical property similar to that of the first amino acid; similar properties include, for example, similar size, charge, hydrophobicity or hydrogen-bonding capacity. Conservative variants are also referred to herein as “conservative amino acid substitutions,” “conservative amino acid variants,” “conservative substitutions,” and similar phrase. A “conservative derivative” of a reference sequence refers to an amino acid sequence that differs from the reference sequences only in conservative substitutions.

As an example, a conservative variant can be a sequence in which a first uncharged polar amino acid is conservatively substituted with a second (non-identical) uncharged polar amino acid such as cysteine, serine, threonine, tyrosine, glycine, glutamine or asparagine or an analog thereof. A conservative variant also can be a sequence in which a first basic amino acid is conservatively substituted with a second basic amino acid such as arginine, lysine, histidine, 5-hydroxylysine, N-methyllysine or an analog thereof. Similarly, a conservative variant can be a sequence in which a first hydrophobic amino acid is conservatively substituted with a second hydrophobic amino acid such as alanine, valine, leucine, isoleucine, proline, methionine, phenylalanine or tryptophan or an analog thereof. In the same way, a conservative variant can be a sequence in which a first acidic amino acid is conservatively substituted with a second acidic amino acid such as aspartic acid or glutamic acid or an analog thereof; a sequence in which an aromatic amino acid such as phenylalanine is conservatively substituted with a second aromatic amino acid or amino acid analog, for example, tyrosine; or a sequence in which a first relatively small amino acid such as alanine is substituted with a second relatively small amino acid or amino acid analog such as glycine or valine or an analog thereof. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein. It is understood that conservative variants of the disclosed amino acid sequences can encompass sequences containing, for example, one, two, three, four or more amino acid substitutions relative to the reference sequence, and that such variants can include naturally and non-naturally occurring amino acid analogs.

Substitutional variants are those in which at least one residue has been removed and a different residue inserted in its place. Examples of such substitutions, referred to as conservative substitutions, can generally be made in accordance with the following Table 2.

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity can be made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation. These can be referred to a less conservative variants.

Peptides can have a variety of modifications. Modifications can be used to change or improve the properties of the peptides. For example, the disclosed peptides can be N-methylated, O-methylated, S-methylated, C-methylated, or a combination at one or more amino acids.

The amino and/or carboxy termini of the disclosed peptides can be modified. Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or .alpha.-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound. In preferred embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the disclosed peptides, or incorporate a desamino or descarboxy residue at the termini of the peptide, so that there is no terminal amino or carboxyl group, to decrease susceptibility to proteases or to restrict the conformation of the peptide. C-terminal functional groups of the disclosed peptides include amide, amide lower alkyl, amide di(lower alkyl), lower alkoxy, hydroxy, and carboxy, and the lower ester derivatives thereof, and the pharmaceutically acceptable salts thereof.

One can replace the naturally occurring side chains of the genetically encoded amino acids (or the stereoisomeric D amino acids) with other side chains, for instance with groups such as alkyl, lower (C₁₋₆) alkyl, cyclic 4-, 5-, 6-, to 7-membered alkyl, amide, amide lower alkyl amide di(lower alkyl), lower alkoxy, hydroxy, carboxy and the lower ester derivatives thereof, and with 4-, 5-, 6-, to 7-membered heterocyclic. In particular, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

One can also readily modify peptides by phosphorylation, and other methods [e.g., as described in Hruby, et al. (1990) Biochem J. 268:249-262].

The disclosed peptides also serve as structural models for non-peptidic compounds with similar biological activity. Those of skill in the art recognize that a variety of techniques are available for constructing compounds with the same or similar desired biological activity as the lead peptide compound, but with more favorable activity than the lead with respect to solubility, stability, and susceptibility to hydrolysis and proteolysis [See, Morgan and Gainor (1989) Ann. Rep. Med. Chem. 24:243-252]. These techniques include, but are not limited to, replacing the peptide backbone with a backbone composed of phosphonates, amidates, carbamates, sulfonamides, secondary amines, and N-methylamino acids.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as β-alanine, γ-aminobutyric acid, and the like.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g. Arg, can be accomplished, for example, by deleting one of the basic residues or substituting one by glutaminyl or histidyl residues.

Certain post-translational derivatizations can be the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed amino acids sequences, amino acid segments, peptides, proteins, etc. herein is through defining the variants and derivatives in terms of homology/identity to specific known sequences. For example, specifically disclosed are variants of these and other amino acids sequences, amino acid segments, peptides, proteins, etc. herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative variants and homology can be combined together in any combination, such as embodiments that have at least 70% homology to a particular sequence wherein the variants are conservative variants.

As this specification discusses various amino acids sequences, amino acid segment sequences, peptide sequences, protein sequences, etc., it is understood that nucleic acids that can encode those sequences are also disclosed. This would include all degenerate sequences related to a specific amino acid sequence, i.e. all nucleic acids having a sequence that encodes one particular amino acid sequence as well as all nucleic acids, including degenerate nucleic acids, encoding the disclosed variants and derivatives of the amino acid sequences. Thus, while each particular nucleic acid sequence may not be written out herein, it is understood that each and every sequence is in fact disclosed and described herein through the disclosed amino acid sequences.

Also disclosed are bifunctional peptides, which contain the homing peptide fused to a second peptide having a separate function. Such bifunctional peptides have at least two functions conferred by different portions of the full-length molecule and can, for example, display anti-angiogenic activity or pro-apoptotic activity in addition to the ability to home to a target.

Also disclosed are isolated multivalent peptides that include at least two subsequences each independently containing a peptide or amino acid segment. The multivalent peptide can have, for example, at least three, at least five or at least ten of such subsequences each independently containing a peptide. In particular embodiments, the multivalent peptide can have two, three, four, five, six, seven, eight, nine, ten, fifteen or twenty identical or non-identical subsequences. This is in addition to the multiple homing molecules and, for example, multiple membrane disrupting molecules that can comprise the disclosed compositions. In a further embodiment, the multivalent peptide can contain identical subsequences, such as repeats of a specified amino acid sequence. In a further embodiment, the multivalent peptide contains contiguous identical or non-identical subsequences, which are not separated by any intervening amino acids.

As used herein, the term “peptide” is used broadly to mean peptides, proteins, fragments of proteins and the like. The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids and have an activity such as selective interaction with a target of the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

A variety of peptidomimetics are known in the art including, for example, peptide-like molecules which contain a constrained amino acid, a non-peptide component that mimics peptide secondary structure, or an amide bond isostere. A peptidomimetic that contains a constrained, non-naturally occurring amino acid can include, for example, an α-methylated amino acid; α,α-dialkylglycine or α-aminocycloalkane carboxylic acid; an N^{α}-C^{α} cyclized amino acid; an N^{α}.-methylated amino acid; a β- or γ-amino cycloalkane carboxylic acid; an α,β-unsaturated amino acid; a β,β-dimethyl or β-methyl amino acid; a β-substituted-2,3-methano amino acid; an N—C^{ε} or C^{α}—C^{Δ} cyclized amino acid; a substituted proline or another amino acid mimetic. A peptidomimetic which mimics peptide secondary structure can contain, for example, a non-peptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylene-sulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

Methods for identifying a peptidomimetic are well known in the art and include, for example, the screening of databases that contain libraries of potential peptidomimetics. As an example, the Cambridge Structural Database contains a collection of greater than 300,000 compounds that have known crystal structures (Allen et al., Acta Crystalloqr. Section B, 35:2331 (1979)). This structural depository is continually updated as new crystal structures are determined and can be screened for compounds having suitable shapes, for example, the same shape as a disclosed peptide, as well as potential geometrical and chemical complementarity to a target molecule. Where no crystal structure of a peptide or a target molecule that binds the peptide is available, a structure can be generated using, for example, the program CONCORD (Rusinko et al., J. Chem. Inf. Comput. Sci. 29:251 (1989)). Another database, the Available Chemicals Directory (Molecular Design Limited, Information Systems; San Leandro Calif.), contains about 100,000 compounds that are commercially available and also can be searched to identify potential peptidomimetics of a peptide, for example, with activity in selectively interacting with cancerous cells.

### G. Pharmaceutical Compositions and Carriers

The disclosed compositions can be administered in vivo either alone or in a pharmaceutically acceptable carrier. By “pharmaceutically acceptable” is meant a material that is not biologically or otherwise undesirable, i.e., the material can be administered to a subject, along with the composition disclosed herein, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution, suspension (for example, incorporated into microparticles, liposomes, or cells).

1. Pharmaceutically Acceptable Carriers

The compositions disclosed herein can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

H. Compositions with Similar Functions

It is understood that the compositions disclosed herein have certain functions, such as binding to clots or enhancing clot formation. Disclosed herein are certain structural requirements for performing the disclosed functions, and it is understood that there are a variety of structures which can perform the same function which are related to the disclosed structures, and that these structures will ultimately achieve the same result, for example stimulation or inhibition.

### I. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits can include the compositions disclosed herein.

### J. Mixtures

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### K. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

### L. Peptide Synthesis

The compositions disclosed herein and the compositions necessary to perform the disclosed methods can be made using any method known to those of skill in the art for that particular reagent or compound unless otherwise specifically noted.

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides can be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

### Methods

Disclosed are methods useful for delivering significant amounts of compounds o interest to targeted cells and tissues. The disclosed methods are useful, for example, to deliver to targeted cells and tissues an effective amount of compounds that are excessively toxic. Also disclosed are methods comprising, for example, administering to a subject the disclosed compositions. Also disclosed are methods of detecting, measuring, imaging, etc. cells and tissues comprising, for example, administering to a subject the disclosed compositions and detecting, measuring, imaging, etc. the composition.

The homing molecules can home to targets of interest, such as cells and tissues of interest. For example, the homing molecules can home to tumor vasculature. The homing molecules can selectively home to targets of interest, such as cells and tissues of interest. For example, the homing molecules can selectively homes to tumor vasculature. The composition can home to one or more of the sites to be targeted. The composition can be internalized in cells. The composition can penetrate tissue. The composition can be internalized into cells at the targeted site. The composition can be penetrate tissue at the targeted site. The composition can, for example be internalized into cancer cells. The composition can, for example, penetrate tumor tissue. The composition can, for example, bind inside tumor blood vessels.

In some forms, the composition can have a therapeutic effect. In some forms, the composition can reduce tumor growth. In some forms, the therapeutic effect can be a slowing in the increase of or a reduction of tumor burden. In some forms, the therapeutic effect can be a slowing of the increase of or reduction of tumor size. In some forms, the subject can have one or more sites targeted, wherein the composition can home to one or more of the sites targeted. In some forms, the subject can have a tumor, wherein the composition can have a therapeutic effect on the tumor.

In some forms, the composition can further comprise one or more internalization elements. In some forms, one or more of the homing molecules can comprise one or more of the internalization elements. In some forms, one or more of the membrane perturbing molecules can comprise one or more of the internalization elements. In some forms, the surface molecule can comprise one or more of the internalization elements not comprised in either the homing molecules or the membrane perturbing molecules. In some forms, the composition can further comprise one or more tissue penetration elements. In some forms, one or more of the tissue penetration elements can be comprised in an internalization element. In some forms, the tissue penetration element can be a CendR element.

In some forms, the composition can further comprise one or more moieties. In some forms, the moieties can be independently selected from the group consisting of an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, an image contrast agent, a fluorophore, fluorescein, rhodamine, a radionuclide, indium-111, technetium-99, carbon-11, and carbon-13. In some forms, at least one of the moieties can be a therapeutic agent. In some forms, the therapeutic agent can be iRGD, RGD, Abraxane, paclitaxel, taxol, or a combination. In some forms, at least one of the moieties can be a detectable agent. In some forms, the detectable agent can be FAM.

In some forms, the composition can have a therapeutic effect. This can be achieved by the delivery of therapeutic cargo molecules to the target site. The therapeutic effect can be a slowing in the increase of or a reduction of tumor burden. This slowing in the increase of, or reduction in the tumor burden, can be 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900%, or 1000% or more improvement in the increase of, or reduction in the tumor burden of, compared with a non-treated tumor, or a tumor treated by a different method.

The disclosed compositions can be used to treat any disease where uncontrolled cellular proliferation occurs such as cancers. A non-limiting list of different types of cancers can be as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

The disclosed compositions can also be administered following decoy particle pretreatment to reduce uptake of the compositions by reticuloendothelial system (RES) tissues. Such decoy particle pretreatment can prolong the blood half-life of the particles and increases tumor targeting.

The method can further comprise, following administering, detecting the disclosed compositions. The disclosed compositions can be detected by fluorescence, CT scan, PET or MRI. The disclosed compositions can be detected by fluorescence. The disclosed compositions can conjugate with tumor vasculature or a tumor in a subject.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “subject” includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. In particular, pets and livestock can be a subject. The subject can be an invertebrate, such as a worm or an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In the context of endometriosis and endometriosis cells, it is understood that a subject is a subject that has or can have endometriosis and/or endometriosis cells.

In one aspect, the compounds described herein can be administered to a subject comprising a human or an animal including, but not limited to, a mouse, dog, cat, horse, bovine or ovine and the like, that is in need of alleviation or amelioration from a recognized medical condition.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

By “pharmaceutically acceptable” is meant a material that is not biologically or otherwise undesirable, i.e., the material can be administered to an individual along with the selected compound without causing any undesirable biological effects or interacting in a deleterious manner with any of the other components of the pharmaceutical composition in which it is contained.

Any of the compounds having the formula I can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### A. Example 1

### Nanoparticle Homing to Tumors

Glioblastomas multiforme (GBM) are the most common and lethal form of intracranial tumors. They account for approximately 70% of the 22,500 new cases of malignant primary brain tumors that are diagnosed in adults in the United States each year. Although relatively uncommon, malignant gliomas are associated with disproportionately high morbidity and mortality (median survival is only 12 to 15 months). Malignant gliomas are among the most vascular of human tumors. Tumor vasculature has proven to be particularly well suited as a site for receptor-based targeting. It expresses a multitude of molecules that are not expressed in the vessels of normal tissues. A peptide, CGKRK (Hoffman, J. A., et al. Progressive vascular changes in a transgenic mouse model of squamous cell carcinoma. Cancer Cell 4, 383-391 (2003)), binds to the blood vessels in various kinds of tumors. Experiments showed that intravenously injected CGKRK peptide effectively homes to lentiviral (H-RasV12-sip53)-induced glioma in mice. The CGKRK peptide was coupled to the alpha-helical amphipathic peptide _{D}[KLAKLAK]₂, which is toxic to eukaryotic cells if it internalized into the cells (Ellerby, H. M., et al. Anti-cancer activity of targeted pro-apoptotic peptides. Nature Medicine 5, 1032-1038 (1999)). The chimeric peptide, when added to actively growing human umbilical vein endothelial cells (HUVEC) or U87 glioma cells colocalized with mitochondria, whereas _{D}[KLAKLAK]₂ did not. Iron oxide nanoworms (NW) coated with the chimeric _{D}[KLAKLAK]₂-CGKRK peptide were 100-200-fold more toxic to the HUVEC and U87 cells than the free peptide in vitro and specifically accumulated in the blood vessels of glioma. Treatment of lentiviral induced glioma in mice with the _{D}[KLAKLAK]₂-CGKRK-PEG-NW inhibited tumor growth and showed a significant survival increase compared to control-treated mice.

Mice bearing brain tumors (RAS-sip53 induced brain tumors into the right hippocampus) were intravenously injected with 200 microg of FAM-labeled CGKRK peptides and allowed to circulate for 3 hours. The mice were perfused through the heart with PBS, and the organs were collected. CGKRK peptide accumulated in glioblastoma compared to normal brain tissue. The dotted line outlines the tumor. Magnification ×200.

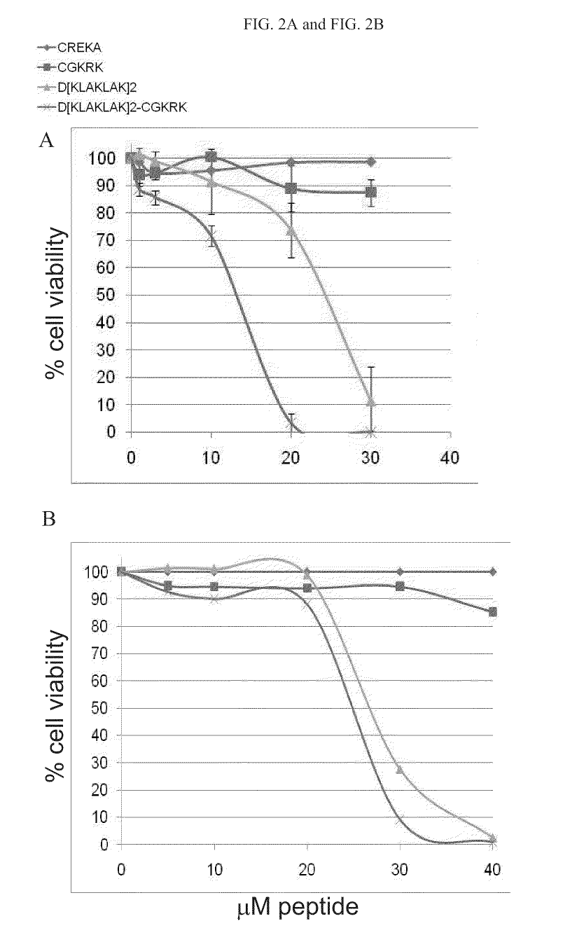

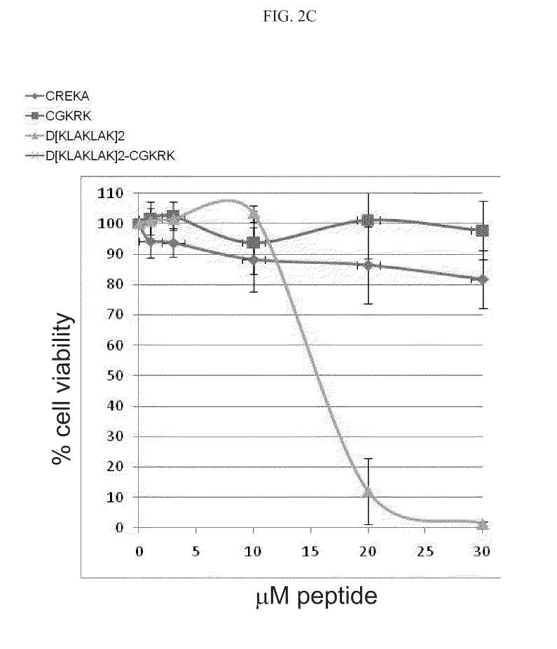

Cultured cells were treated CGKRK, _{D}(KLAKLAK)₂, or _{D}(KLAKLAK)₂CGKRK peptide. The cells were incubated with peptide for 24 hrs and cell death was quantified by MTT assays (n=3). FIGS. 2A, 2B and 2C show cytotoxicity of _{D}(KLAKLAK)₂CGKRK peptide in cell lines. FIGS. 2A and 2B show the cytotoxicity of _{D}(KLAKLAK)₂CGKRK peptide in HUVEC (A) and T3 (B) cells. Statistical analyses were performed with Student's t-test. Error bars, s.e.m.

Confocal microscopic images of HUVEC cells incubated for 2 h at 37° C. with KAKEC-NW (SEQ ID NO:135), or CGKRK-NR, or _{D}(KLAKLAK)₂—NR, or _{D}(KLAKLAK)₂CGKRK-NW were prepared. Subcellular localization of nanoworms was identified in HUVEC cells. There is a high sub-colocalization of CGKRK-NW and _{D}(KLAKLAK)₂CGKRK-NW with mitochondria marker.

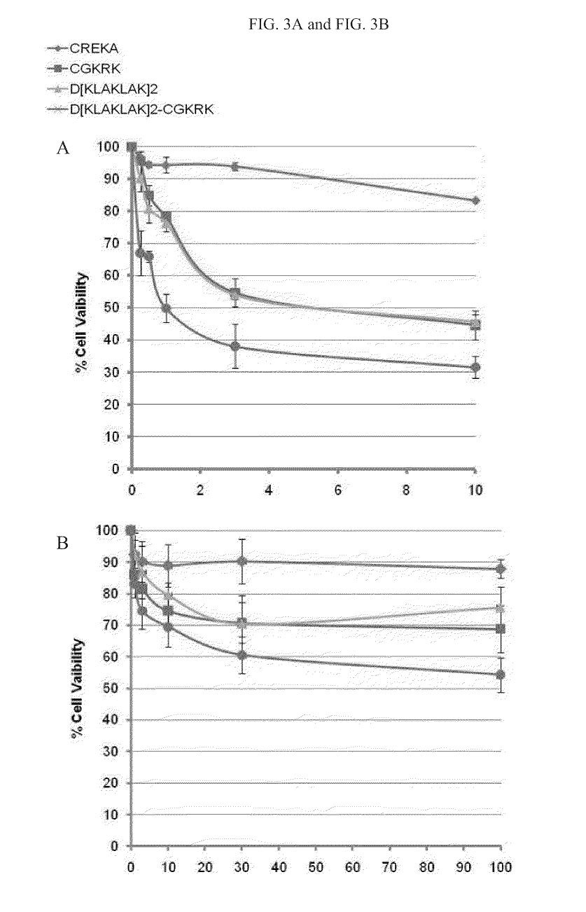

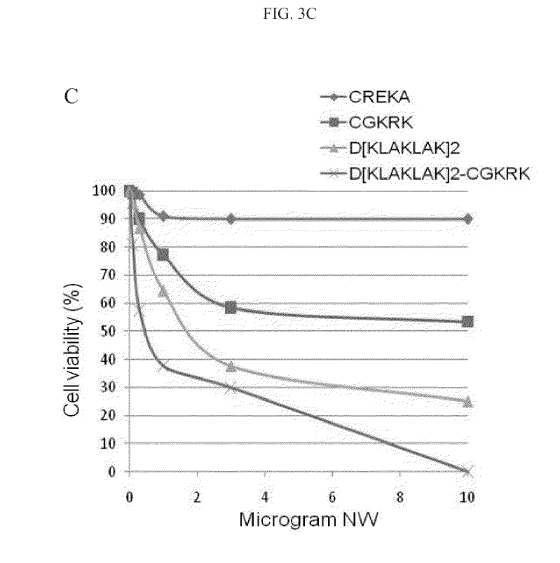

Confocal microscopic images of HUVEC cells incubated for 2 h at 37° C. with CGKRK-NW or _{D}(KLAKLAK)₂CGKRK-NW were prepared. Competition of subcellular localization of nanoworms in HUVEC cells was seen with both CGKRK-NW and _{D}(KLAKLAK)₂CGKRK-NW treated cells. 10× non-labeled peptide-NW was added to the cells 15 min before adding the labeled peptide-NR. Cultured HUVEC cells were treated with non-targeted _{D}(KLAKLAK)₂ conjugated NW (_{D}(KLAKLAK)₂-NW), CREKA conjugated NW (CREKA-NW), CGKRK conjugated NW (CGKRK-NW), or CGKRK-_{D}(KLAKLAK)₂ conjugates NW (_{D}(KLAKLAK)₂-CGKRK-NW). FIGS. 3A and 3B show cytotoxicity of _{D}(KLAKLAK)₂CGKRK conjugated with NW in HUVEC cells. The cells were incubated with NW for 48 hrs without washing (A) or the NW were washed after 20 min (B) and cell death was quantified by MTT assays (n=3). Statistical analyses were performed with Student's t-test. Error bars, s.e.m.

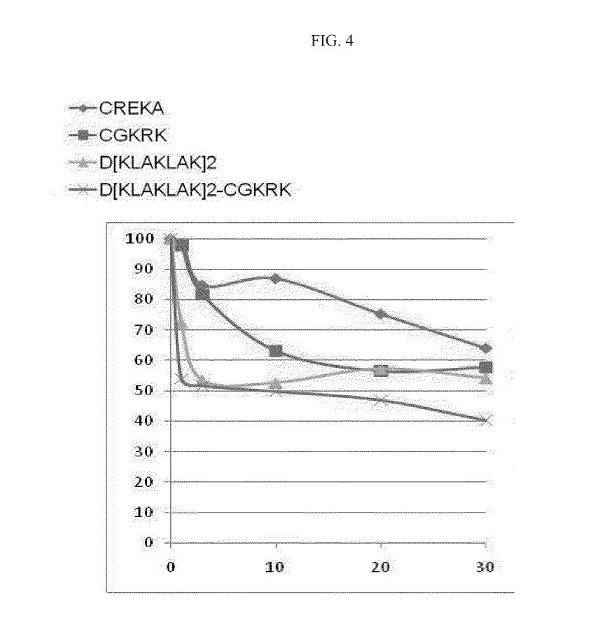

Cultured T3 cells were treated with non-targeted _{D}(KLAKLAK)₂ conjugated NW (_{D}(KLAKLAK)₂-NW), CREKA conjugated NW (CREKA-NW), CGKRK conjugated NW (CGKRK-NW), or _{D}(KLAKLAK)₂CGKRK conjugated with NW (_{D}(KLAKLAK)₂CGKRK-NW). The cells were incubated with NW for 48 hrs and cell death was quantified by MTT assay. FIG. 4 shows cytotoxicity of _{D}(KLAKLAK)₂CGKRK conjugated with NW in T3 cells.

Confocal microscopic images of U87 cells incubated for 2 h at 37° C. with CGKRK-NR, or _{D}(KLAKLAK)₂-NR, or CGKRK-_{D}(KLAKLAK)₂-NW (Chimera-NW) were prepared. Subcellular localization of nanoworms was identified in U87 cells. There is high sub-colocalization of CGKRK-NW and _{D}(KLAKLAK)₂CGKRK-NW with mitochondria.

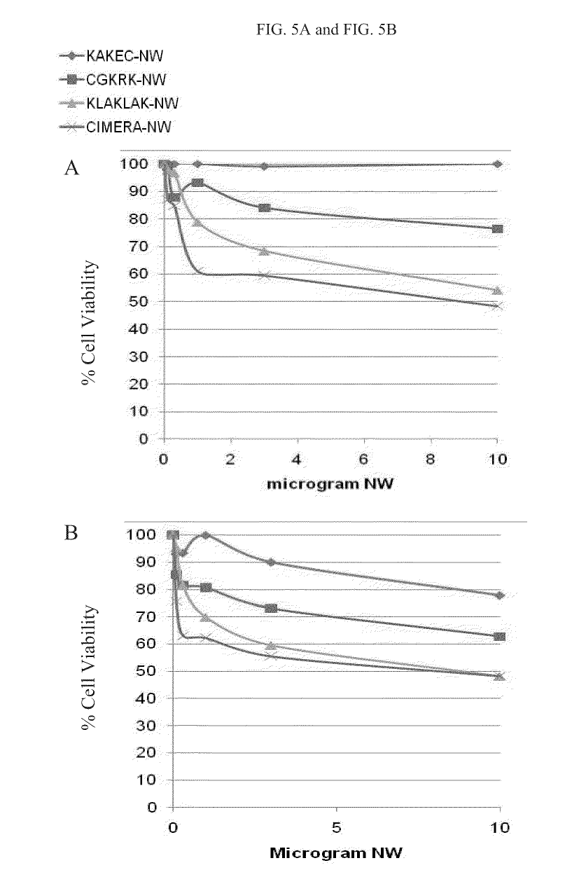

Cultured U87 cells were treated with non-targeted _{D}(KLAKLAK)₂ conjugated NR, KAKEC (SEQ ID NO:135) conjugated NW (KAKEC-NW), CGKRK conjugated NW (CGKRK-NW), or CGKRK-_{D}(KLAKLAK)₂ conjugated with NW (_{D}(KLAKLAK)₂CGKRK-NW). The cells were incubated with NW for 24 or 48 hrs and cell death was quantified by MTT assays. FIG. 5 shows cytotoxicity of _{D}(KLAKLAK)₂CGKRK conjugated with NW in U87 cells. These results are almost the same results seen with U251 which had 50-60% cell viability.

NW coated with _{D}(KLAKLAK)₂CGKRK via a 5-kDa PEG-linker were cleaved from the particles using DTT and the amount of peptide present on the particle was calculated to compare the amount of free peptide versus the peptide coated nanoparticle IC50 values. FIG. 6 shows the IC50 of _{D}(KLAKLAK)₂CGKRK peptide versus peptide on nanoworms.

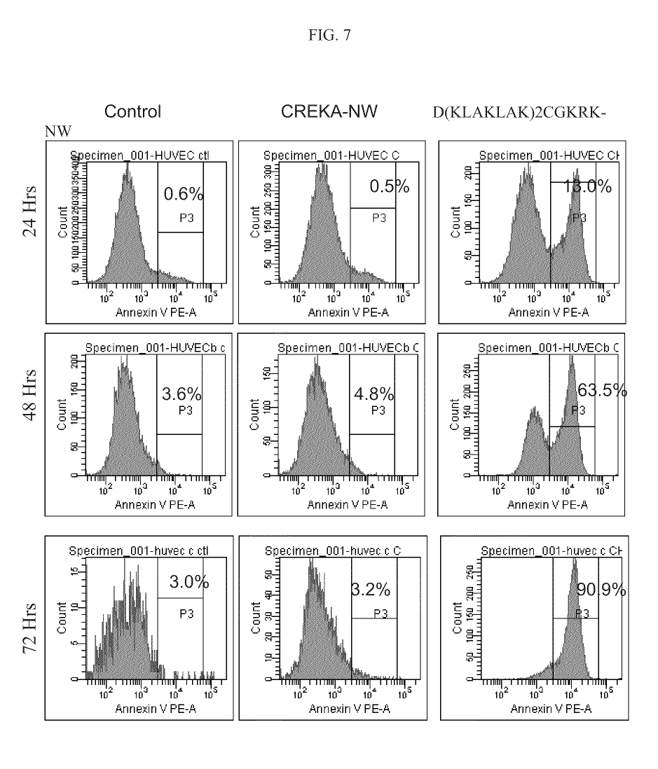

HUVEC cells were left untreated (Control) or treated for 24, 48 and 72 hrs with an irrelevant peptide-NW (CREKA-NW) or the _{D}(KLAKLAK)₂CGKRK-NR. Cells were incubated with Annexin V-PE in a buffer containing 7-Amino-actinomycin (7-AAD) and analyzed by flow cytometry. FIG. 7 shows _{D}(KLAKLAK)₂CGKRK conjugated with NW induced apoptosis in HUVEC cells. The percentage of Annexin V positive cells (apoptotic cells plus end stage apoptosis or already dead cells) is indicated in each graph.

T3 cells (tumor endothelial cells) were left untreated (Control) or treated for 24 and 48 hrs with an irrelevant peptide-NW (CREKA-NW) or the _{D}(KLAKLAK)₂CGKRK-NR. Cells were incubated with Annexin V-PE in a buffer containing 7-Amino-actinomycin (7-AAD) and analyzed by flow cytometry. FIG. 8 shows _{D}(KLAKLAK)₂CGKRK conjugated with NW induced apoptosis in T3 cells. The percentage of Annexin V positive cells (apoptotic cells plus end stage apoptosis or already dead cells) is indicated in each graph.

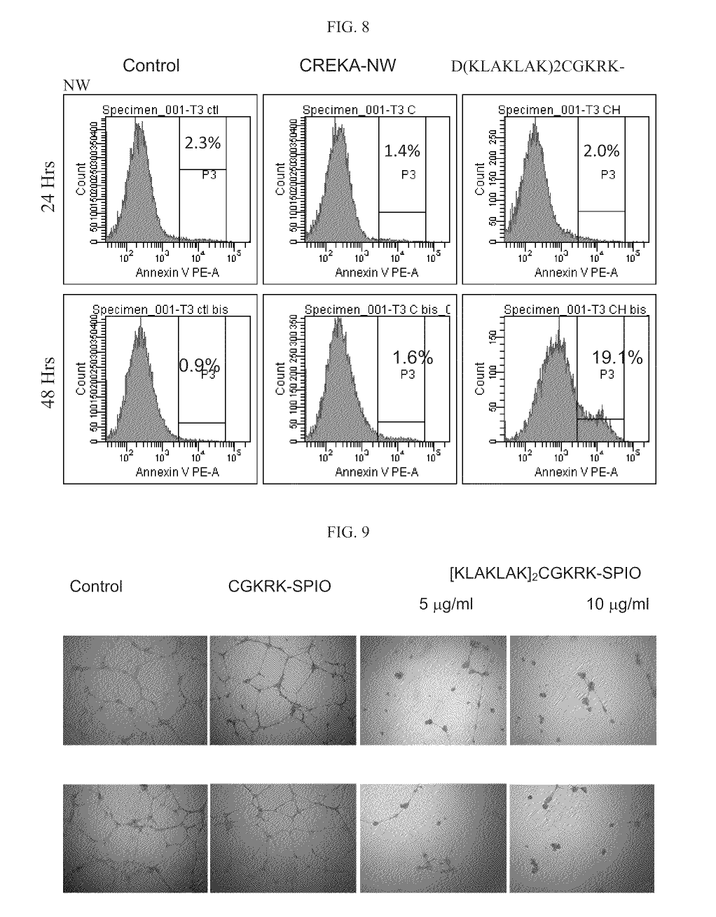

Primary HUVECs were plated on growth factor reduced matrigel in 5% FCS medium alone (control), or containing CGKRK-NW (10 microg/ml), or containing _{D}(KLAKLAK)₂CGKRK-NW (5 and 10 microg/ml). The formation of networks of capillary-like structures was viewed by phase contrast-microscopy at 40× magnification 24 h after plating. FIG. 9 shows _{D}(KLAKLAK)₂CGKRK conjugated with NW inhibits HUVEC capillary-like tube formation in vitro.

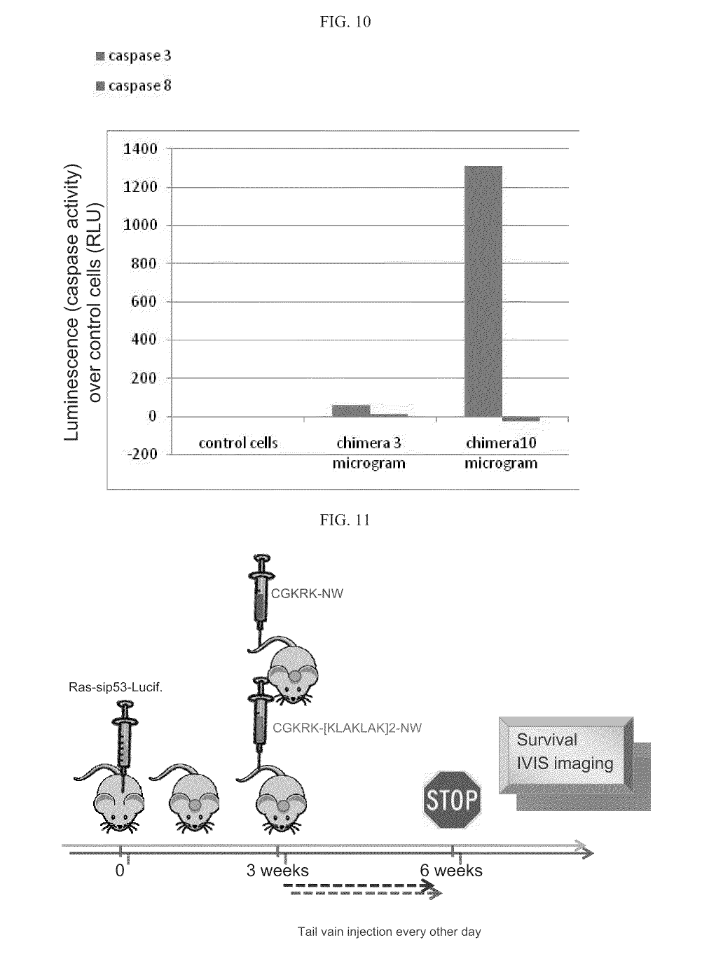

Caspase-3 activity was determined in HUVEC cells 24 h after treatment with 3 or 10 microgram _{D}(KLAKLAK)₂CGKRK-NW using a caspase-Glo 3/7 assay kit. Two hours after reagent was added luminescence was recorded on luminometer. FIG. 10 shows caspase activity by HUVEC cells treated with _{D}(KLAKLAK)₂CGKRK-NR.

HUVEC cells were treated either with CREKA-NW (SEQ ID NO:92) as control (10 μg) or _{D}(KLAKLAK)₂CGKRK-NW (10 μg) for 24, 48 and 72 hr. Whole cell extracts were prepared and analyzed by Western blotting using antibodies against cleaved caspase-3 (exp 1) or caspase 3 (exp 2). _{D}(KLAKLAK)₂CGKRK-NW increased caspase-3 activity.

HUVEC cells were untreated (control), or treated either with CREKA-NW (10 μg), or _{D}(KLAKLAK)₂CGKRK-NW (10 μg) for 24 hr. Confocal microscopy images of HUVEC cells incubated with _{D}(KLAKLAK)₂CGKRK-NW showed increased cleaved caspase-3 in comparison to untreated or CREKA-NW (SEQ ID NO:92) treated cells. Iron oxide NW coated with 5K-PEG-FAM-labeled _{D}(KLAKLAK)₂CGKRK peptide were intravenously injected (5 mg iron per kg body weight) into mice bearing RAS-sip53 induced brain tumors (viral injections into the right hippocampus). Six hours later post-injection, the mice were perfused through the heart with PBS, and the organs were collected. Tumor sections were stained and examined by confocal microscopy. CGKRK-D(KLAKLAK)₂-NW was shown to home to glioblastoma multiforme (GBM). Mice bearing RAS-sip53 induced brain tumors (three weeks post-injection) were intravenously injected with NW coated with peptides through a 5-kDa polyethylene glycol spacer. The particles were administered every other day for 14 days (5 mg iron/kg/day, total cumulative dose 35 mg/kg). Survival was monitored over time (n=3 per group). FIG. 11 is a diagram of the GBM treatment with CGKRK-_{D}(KLAKLAK)₂-NW nanoworms (EXP NUMBER 1).

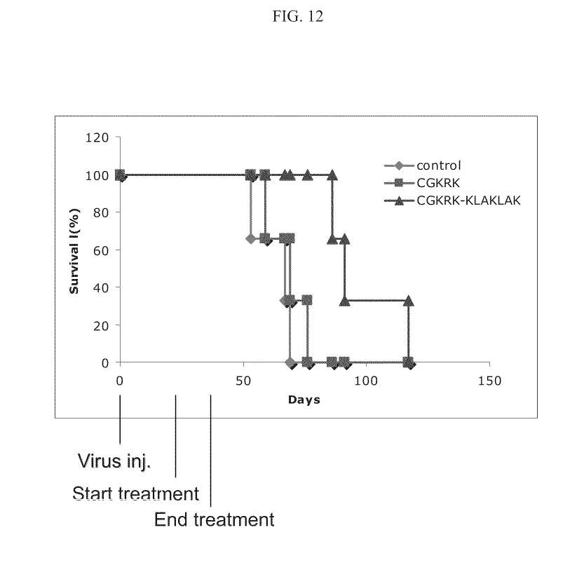

Mice bearing RAS-sip53 induced brain tumors (three weeks post-injection) were intravenously injected with NW coated with peptides through a 5-kDa polyethylene glycol spacer. The particles were administered every other day for 14 days (5 mg iron/kg/day, total cumulative dose 35 mg/kg). Survival was monitored over time (n=3 per group). FIG. 12 shows GBM treatment with CGKRK-_{D}(KLAKLAK)₂-NW nanoworms (EXP NUMBER 1).

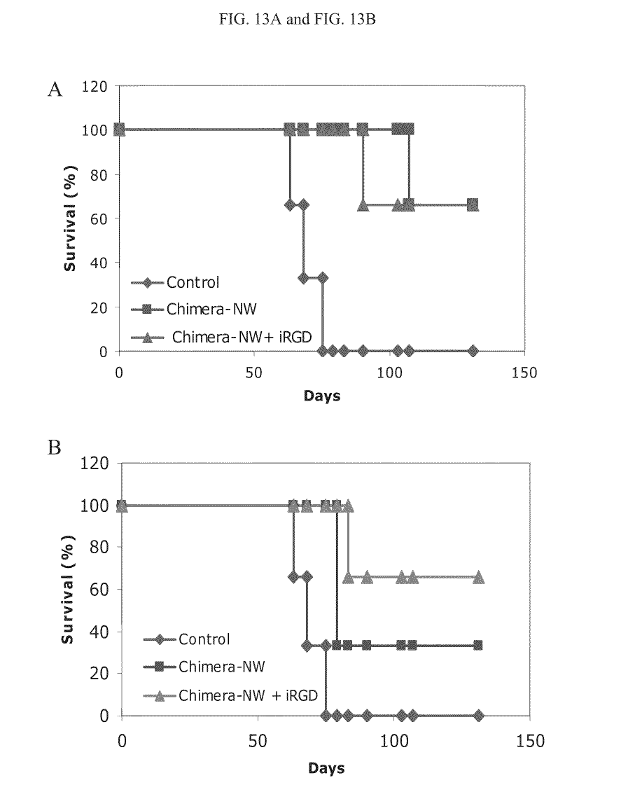

Mice bearing RAS-sip53 induced brain tumors (injection to the right hippocampus) were intravenously injected with NW coated with peptides through a 5-kDa polyethylene glycol spacer. The particles alone or co-injection with iRGD were administered once a week for 6 weeks (one weeks post-viral injection), or every other day for two weeks and a half weeks (three weeks post-viral injection) via tail vein injection. All mice were monitored for luciferase signal using the IVIS system (the lentivector contains the luciferase reporter). FIGS. 13A and 13B show GBM treatment with CGKRK-_{D}(KLAKLAK)₂-NW nanoworms (EXP NUMBER 2). Survival of the mice is being currently recorded (n=3 per group).

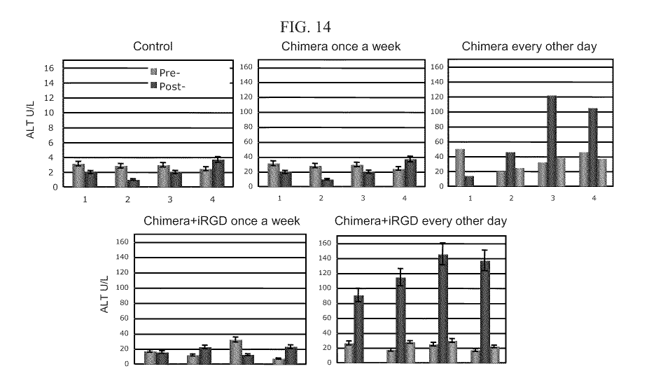

Mice were bled one day before starting the treatment and one day following the two and a half treatment course. For the groups of mice injected every other day another blood collection was performed two weeks after the last day of treatment. The levels of ALT were tested in the serum of all the mice. Normal values go from 10-40 U/L. FIG. 14 shows ALT (L-Alanine-2-Oxoglutarate Aminotransferase) levels in mice pre and post-nanoworm treatment.

Mice bearing RAS-sip53 induced brain tumors (injection to the right hippocampus) were intravenously injected with NW coated with peptides through a 5-kDa polyethylene glycol spacer. Confocal immunofluorescent analysis of frozen RAS-sip53 induced brain tumors. One mouse from each of the indicated groups (left side) was euthanized and frozen sections were prepared from the brain. NW distribution after tumor therapy showed the presence of NW coated peptides in the tumor. Confocal images of normal organs from mice bearing RAS-sip53 induced brain tumors injected with FAM-_{D}(KLAKLAK)₂CGKRK-NW were taken. The distribution of FAM-D(KLAKLAK)₂CGKRK-NW in normal organs (in the end of the treatment) showed the presence of FAM-_{D}(KLAKLAK)₂CGKRK-NW in the kidney and spleen. The kidney and spleen were the only non-tumor tissues that showed significant _{D}(KLAKLAK)₂CGKRK-NW fluorescence. Presence of the chimera peptide-NW in the spleen is due to general uptake of nanoparticles unrelated to the homing peptide and kidney is due to cleavage of the peptide from the particle.

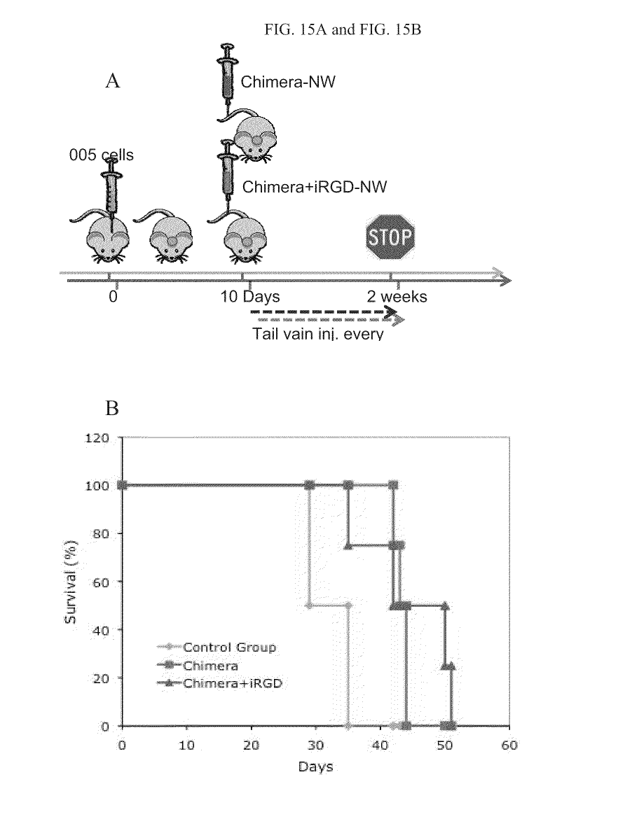

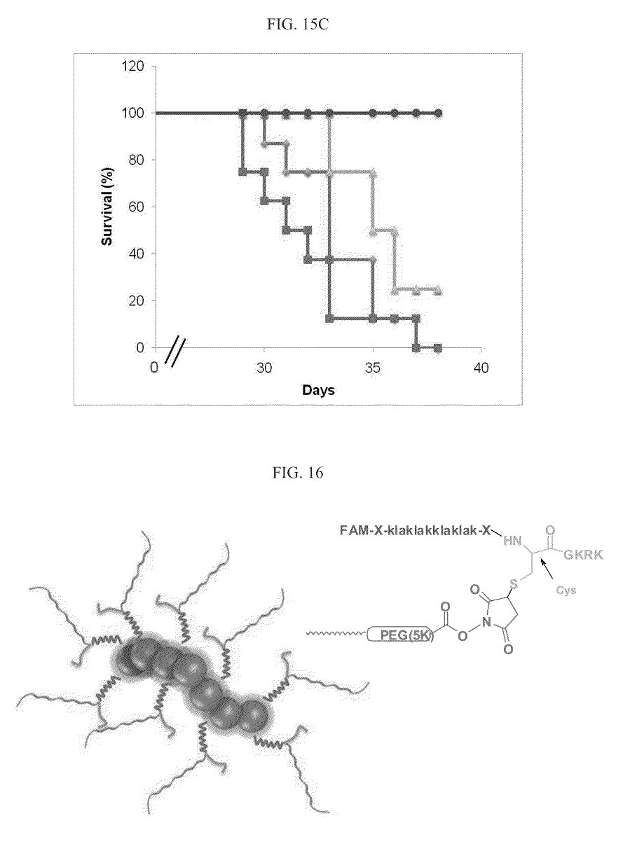

FIGS. 15A and 15B show the GBM treatment with CGKRK-_{D}(KLAKLAK)₂-NW nanoworms. Panel A shows a schematic of the experiment. Mice bearing 005 brain tumor cells (10 day post-injection) were intravenously injected with NW coated with peptides through a 5-kDa polyethylene glycol spacer. The particles without and co-injection with iRGD were administered every other day for 14 days (5 mg iron/kg/day, total cumulative dose 35 mg/kg). Panel B shows a graph of survival. Survival was monitored over time (n=3 per group).

_{D}(KLAKLAK)₂CGKRK-NW were intravenously injected into mice bearing U87. The particles were allowed to circulate for 6 hours (the time determined in preliminary experiments to be optimal for differential homing). The MR Image of _{D}(KLAKLAK)₂CGKRK-NW in U87 T2-weighted MR images (Fast Spin Echo, TR=6.4s, TE=69 ms) shows hypointense vascular signals throughout the tumor. Nontargeted nanoworms gave no detectable signal in these tumors after most of the nanoparticles had been cleared from the blood.

### B. Example 2

### Homing, Localization, and Effect of Homing Molecule Compositions on Glioblastoma

This example describes examples of the disclosed tumor-homing nanoparticle conjugates, which have been constructed based on three novel elements: (1) A tumor-homing peptide that specifically delivers its payload to the mitochondria of tumor endothelial cells and tumor cells; (2) conjugation of this homing peptide with a pro-apoptotic peptide that acts on mitochondria; and (3) coupling of the chimeric peptide onto iron oxide nanoparticles, which greatly enhances the pro-apoptotic activity. Treatment of glioblastoma (GBM)-bearing mice with the nanoparticles eradicated most tumors in one GBM model and significantly delayed tumor development in a more aggressive model. The iron oxide component of the nanoparticles enabled imaging of the tumors. Finally, co-injecting these theranostic particles with the tumor penetrating peptide iRGD further enhanced the therapeutic effect.

Anti-angiogenic therapy was thought to be a promising therapeutic strategy, particularly for highly vascularized GBM tumors. However, these therapies have not proven effective in GMB. The new nanosystem technology disclosed herein shows an unprecedented efficacy in treating GBM as it eradicated most tumors in one mouse GBM model and greatly delayed the demise of the animals in another, more aggressive model. Both of these models had proven completely resistant to other treatment modalities, including anti-angiogenic agents.

Tumor blood vessels have in the recent years become an important therapeutic target. As a tumor grows, the blood vessels grow with it, and this growth primarily takes place through angiogenesis (Hanahan, 1996; Alitalo and Ferrara). Therefore, inhibiting angiogenesis has become a mainstream therapeutic strategy in cancer treatment. The special features of tumor vasculature also enable another strategy, homing-based (synaphic) delivery of drugs (Ruoslahti, 2010). Tumor blood vessels express various cell surface and extracellular matrix proteins that normal vessels do not express or do so at much lower levels than tumor vessels (Hanahan, 1996; Ruoslahti, 2010). These specific vascular markers are readily available to bind circulating ligands, such as peptides and antibodies (Allen, 2004; Jain, 1986; Ruoslahti, 2002). Drugs attached to such ligands will become concentrated in tumor tissue, improving efficacy and allowing the exposure of normal tissues to be reduced (Ruoslahti, 2010).

Vascular markers can be explored in an unbiased manner by in vivo screening of phage libraries that display random peptide sequences (Pasqualini and Ruoslahti, 1996). This approach has yielded a variety of homing peptides specific for tumor vasculature and tumor cells (Arap, 1998; Laakkonen, 2002; Sugahara, 2009). The pentapeptide CGKRK (Cys-Gly-Lys-Arg-Lys; SEQ ID NO:1) was originally identified by in vivo phage library screening with epidermal tumors (Hoffman, 2003). It recognizes the vessels in most tumors and in matrigel plug angiogenesis assays (Hoffman, 2003). CGKRK is internalized into the target cells and can take a payload with it. Intravenously injected CGKRK into tumor mice specifically accumulates in the tumor localizing in both endothelial cells and tumor cells, but is not detectable in normal tissues (Hoffman, 2003). CGKRK peptide was chosen as the homing peptide for this study because of its excellent targeting specificity, cell internalizing properties, simple structure, and the availability of the sulfhydryl group in the cysteine side chain for conjugation.

The α-helical amphipathic peptide, _{D}[KLAKLAK]₂ (SEQ ID NO:3), was originally designed as a synthetic anti-bacterial peptide that disrupts the bacterial cell membrane, but is less toxic to eukaryotic cells (Javadpour, 1996). However, when internalized into eukaryotic cells, _{D}[KLAKLAK]₂ disrupts the mitochondrial membrane, which is similar to the cell membrane bacteria, initiating apoptotic cell death (Ellerby, 1999). Conjugating _{D}[KLAKLAK]₂ with homing peptides have produced compounds with specifically accumulate at the target of the homing peptide causing cell killing (Ellerby, 1999; Arap, 2002; Gerlag et al.). In this example, a tumor-homing _{D}[KLAKLAK]₂ compound was made by conjugating _{D}[KLAKLAK]₂ to CGKRK.

_{D}[KLAKLAK]₂ is a highly toxic compound, even when specifically targeted to tumors (Arap et al., 2002). Administering toxic drugs in a nanoparticle formulation can reduce toxicity. Examples include paclitaxel-albumin nanoparticles (Abraxane^{R}) and doxorubicin liposomes (Doxil^{R}), both of which are in clinical use. Other advantages of nanoparticles include that compounds coupled onto their surface can be presented in a multivalent fashion, which increases the binding efficiency at the target, and that multiple functions can be built into a nanoparticle. These features of nanoparticles are used in the disclosed compositions and in using the CGKRK-_{D}[KLAKLAK]₂ conjugate. Iron oxide nanoworms (NWs) are useful as the nanoparticle scaffold because, for example, iron oxide can be used as an MRI contrast agent, making the resulting nanoparticle a theranostic compound, a compound with both a therapeutic and diagnostic function.

A common disadvantage of nanoparticles as drugs is that their large size can make it more difficult for them to penetrate from the blood into tissues than is the case with simple molecules, limiting the effects to the vessels and their immediate vicinity. Recently discovered tumor-penetrating peptides can be used with the disclosed compositions to solve this problem. These peptides, an example of which is a 9-amino acid peptide named iRGD (CRGDKGPDC or Cys-Arg-Gly-Asp-Lys-Gly-Pro-Asp-Cys; SEQ ID NO:134). These peptides bind to a primary receptor (av integrins in the case of iRGD), then are proteolytically processed to unmask an R/KXXRK-OH motif which binds to neuropilin-1 activating a transport pathway across the vessel wall and through tissue (Teesalu et al., 2009; Sugahara et al., 2009). A payload does not have to be coupled to the peptide to be transported; the pathway is a bulk transport pathway that will sweep along bystander molecules and nanoparticles (Sugahara et al., 2010). The final element in our CGKRK-_{D}[KLAKLAK]₂-nanoparticle regimen was to combine the nanoparticles with iRGD in tumor therapy.

Glioblastoma (GBM) is the most frequent primary brain tumor in adults and has a poor prognosis. Despite a multi-modality treatment approach, which includes surgery, irradiation, and chemotherapy; the median survival is only 12 months (Wen, 2008). Thus, more effective treatments are desperately needed for this cancer. Here we use the targeted _{D}[KLAKLAK]₂ nanoparticles to treat experimental GBM tumors.

1. Results

i. CGKRK Peptide Homing to Brain Tumor and its Co-localization with Mitochondria in Cells

The CGKRK peptide recognizes endothelial cells and tumor cells in various types of tumors (Hoffman, 2003). CGKRK homing to GBM tumors (Marumoto, 2009; Soda, 2011) was tested for this example. Intravenously injected CGKRK strongly accumulated in GBM tumors, as indicated by the rhodamine label on the peptide, but not in normal tissues. This peptide was used as a targeting agent for the glioblastomas for compositions in this example.

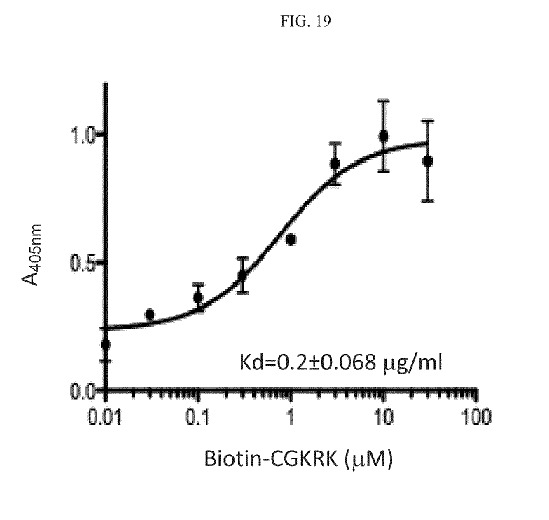

CGKRK has the ability to become internalized into the target cells and take a payload with it (Hoffman, 2003). To evaluate the intracellular localization of CGKRK peptide, live cell imaging was performed with FAM-CGKRK and found it co-localized with a mitochondrial marker in HUVEC and U87, human glioma cells. To determine the specificity of CGKRK to the mitochondria, mitochondria were isolated from liver, incubated with FAM-CGKRK and an excess of either non-labeled CGKRK or control peptide (CREKA; SEQ ID NO:92). The specificity of the CGKRK peptide binding to the mitochondria was competitively inhibited by unlabeled CGKRK but not CREKA (FIG. 17). Furthermore, a phage binding assay to the isolated mitochondria was performed and an 80 fold increase in binding of CGKRK-phage compare to control was found (FIG. 18) indicated that mitochondria are the primary subcellular target organelle of CGKRK peptide.

ii. CGKRK Peptide Binds to p32 Protein.

To identify the target for the CGKRK peptide in mitochondria, CGKRK peptide coupled SulfoLink Resin was incubated with extracts from the mitochondria purified from mouse livers, which we have shown significantly binds CGKRK (FIGS. 17 and 18). Bound proteins were eluted with excess of free CGKRK peptide (2 mM) or CREKA peptide as a control. CGKRK bound a specific band below 36-kDa and was not seen in the controls. The specific band was identified as C1qBP or p32 by mass spectrometry. The identification of the CGKRK-binding protein as p32 was confirmed by immunoblotting.

Saturation binding experiments gave an average binding affinity of K_{d}=0.2 mg/ml for the CGKRK-p32 interaction (FIG. 19). Finally, blocking purified p32 with full length antibody against p32 reduced the binding of biotin CGKRK in a concentration-dependent manner up to 40% (FIG. 26). These results indicate that indeed CGKRK recognizes p32 in the mitochondria.

iii. Intratumoral distribution of iron oxide nanoworms coated with CGKRK_{D}(KLAKLAK)₂

A variety of anti-cancer drugs show an enhanced anti-tumor effect when they are coupled to a tumor-homing peptide (Arap, 2002; Curnis, 2000; Ellerby, 1999; Hamzah, 2008; Karmali, 2009). It was realized that the mitochondria localization of CGKRK provides a way of improving the delivery of a pro-apoptotic peptide. A targeting system was set up that consists of 3 elements: a tumor-homing peptide (CGKRK; Hoffman, 2003), a pro-apoptotic peptide [_{D}[KLAKLAK]₂; Ellerby, 1999 (an example of a membrane perturbing peptide)], and iron oxide nanoparticles dubbed nanoworms (NWs) because of their elongated shape (Agemy, 2010; Park, 2009). The two peptides were synthesized as a chimeric peptide that is covalently linked to the NWs through a 5K-polyethylene glycol (PEG) linker.

Intravenously injected NWs coated with the CGKRK-_{D}[KLAKLAK]₂ chimeric peptide accumulated mainly in tumor vessels of different mouse and human GBM xenograft model tumors (005, Human GBM spheres, and U87). The vessels of the intact brain did not attract CGKRK-_{D}[KLAKLAK]₂-NWs. NWs coated only with CGKRK also accumulated in tumor vessels, whereas _{D}(KLAKLAK)₂-coated NWs did not. No fluorescence from the various NW formulations was observed in normal tissues of the tumor-bearing mice, with the exception of the liver and the spleen, which take up all nanoparticles non-selectively.

To demonstrate use of the iron oxide component in the targeted pro-apoptotic peptide-NW as an MRI contrast agent for clinical applications, magnetic resonance imaging (MRI) was performed. Magnetic resonance imaging of 005 tumors after intravenous injection of CGKRK_{D}[KLAKLAK]₂-NWs showed hypointense vascular signals throughout the tumor.

iv. Targeted Pro-apoptotic Peptide-NW Induces Apoptosis

To evaluate the ability of CGKRK-_{D}[KLAKLAK]₂-NWs to induce apoptosis, the co-localization of the particles with a mitochondrial marker was assessed. CGKRK-NWs and CGKRK-_{D}[KLAKLAK]₂-NWs were taken up into the cells and co-localized with mitochondria whereas only small amounts of _{D}[KLAKLAK]₂-NWs internalized and co-localized with mitochondria. Next, the cell death affectivity of the peptide compared to the targeted-NWs was evaluated. To be able to quantify the amount of peptide that is coupled onto the NWs, FAM-CGKRK-_{D}[KLAKLAK]₂ peptide was coupled onto the NWs via a reducible 5-kDa PEG linker. The linker was cleaved from the NWs and the amount of peptide present on the NWs was determined by UV-spectrophotomer using standard curve for the free FAM peptide and used to calculate the IC₅₀. HUVEC cells, 005 cells trans-differentiated to cancer endothelial cells (T3) (Soda, 2011) and U87 cells were used. Coupling the CGKRK-_{D}[KLAKLAK]₂ peptide to NWs increases the cytotoxicity hundreds of times more than the monomeric peptide.

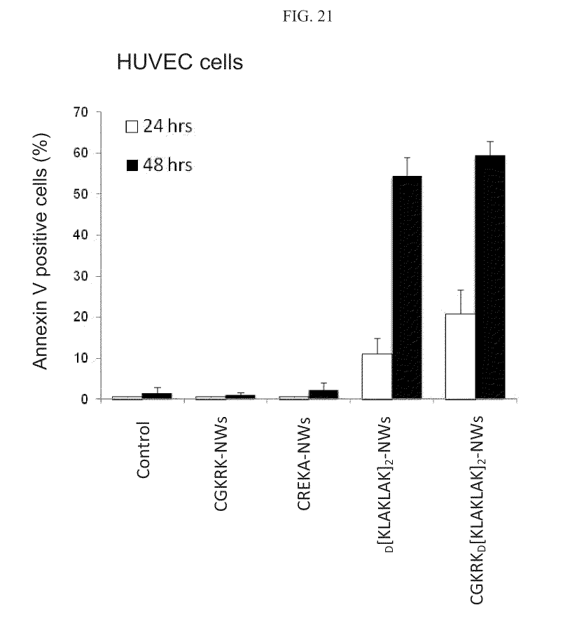

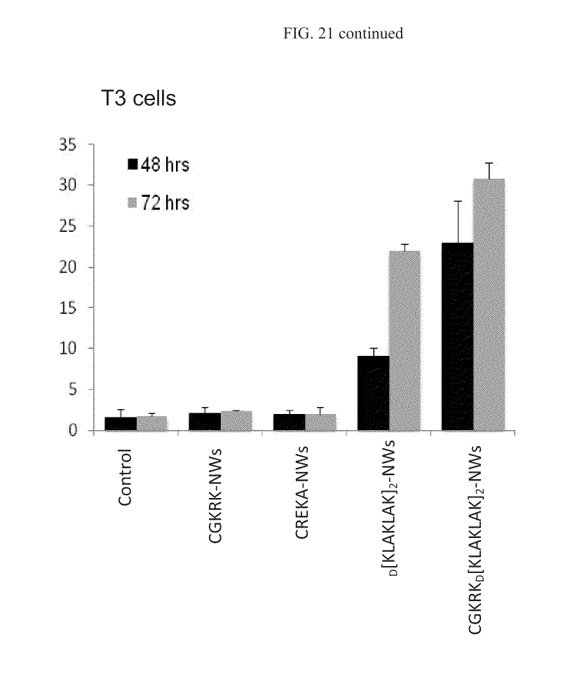

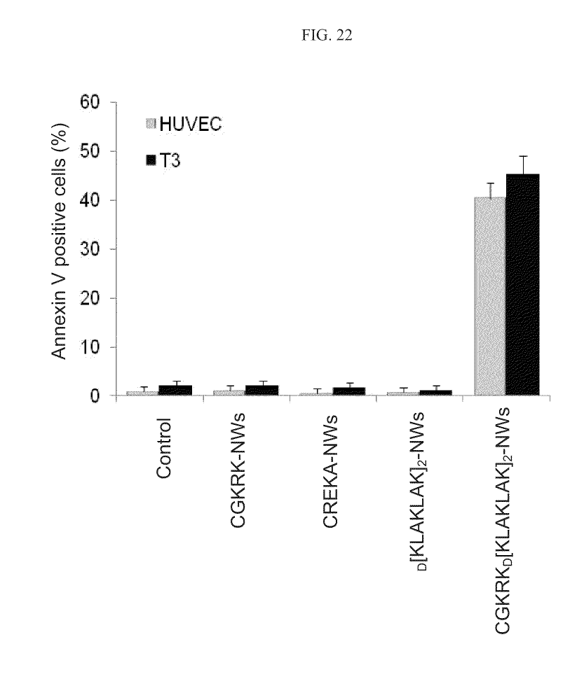

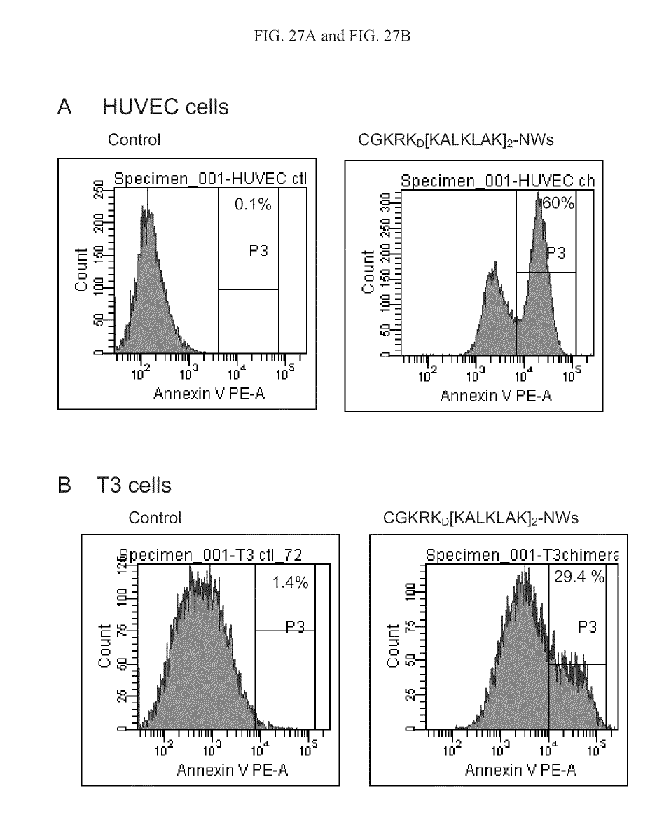

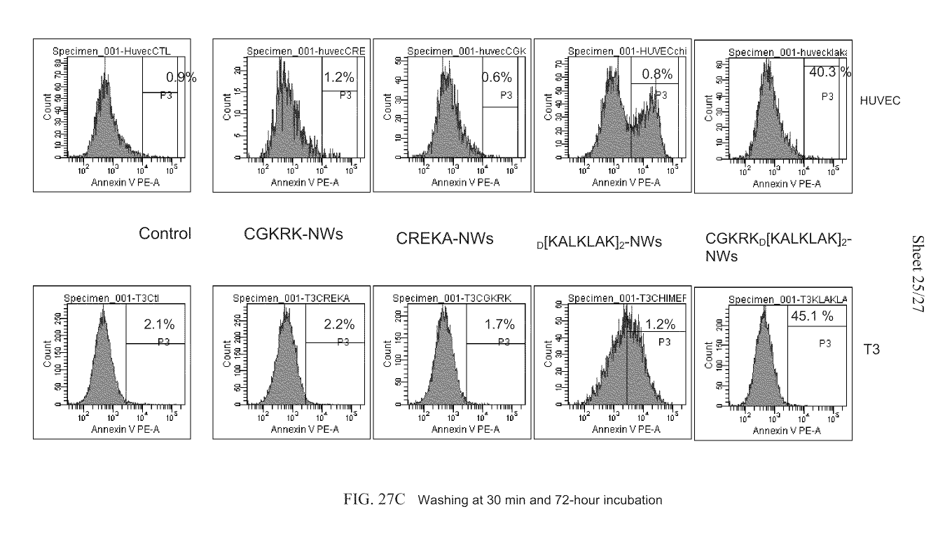

To further analyze this cytotoxicity effect it was checked whether CGKRK-D[KLAKLAK]₂-NWs can lead to cell death via apoptosis as was previously reported for the _{D}[KLAKLAK]₂ coupled to an internalizing peptide (Ellerby, 1999) Annexin V staining confirmed that treatment with pro-apoptotic peptide-NWs induces cell death through apoptosis (FIGS. 21 and 27). A significant increase of apoptotic cells was observed after treatment with CGKRK-_{D}[KLAKLAK]₂-NWs and _{D}[KLAKLAK]₂-NWs in HUVEC (around 60% after 48 hr), and in T3 cells (around 35% after 72 hr). The control particles CREKA-NWs and CGKRK-NWs showed no significant effect. Moreover, when the particles were washed after 30 min of incubation, only CGKRK-_{D}[KLAKLAK]₂-NWs induced significant apoptosis in both types of cells (40-50% after 72 hr), emphasizing the important role of CGKRK as an internalizing peptide (FIG. 22). Furthermore, apoptotic cell death by CGKRK-_{D}[KLAKLAK]₂-NWs induced caspase-3 cleavage.

To study the role of CGKRK-_{D}[KLAKLAK]₂-NW on angiogenic blood vessels in vitro tube formation on HUVEC cells was tested. CGKRK-_{D}[KLAKLAK]₂-NWs significantly reduce the ability of HUVEC to form tube like structure on matrigel whereas CGKRK-NWs have no significant effect. To assess the anti-angiogenic effect in vivo, matrigel plaque assays were employed. Matrigel/bFGF bearing Balb/c nude mice were treated i.v. with either PBS or CGKRK-_{D}[KLAKLAK]₂-NWs. After 14 days, the mice were perfused with cy5-lectin, and the matrigel plugs were excised. Imaging of the matrigel plaque by near-infrared dye and confocal microscopy revealed that treatment with CGKRK-_{D}[KLAKLAK]₂-NWs leads to significant decrease in blood vessel formation compared to the control plaque.

v. Therapeutic Efficacy of Targeted Pro-apoptotic Peptide-coated NW in Glioblastoma

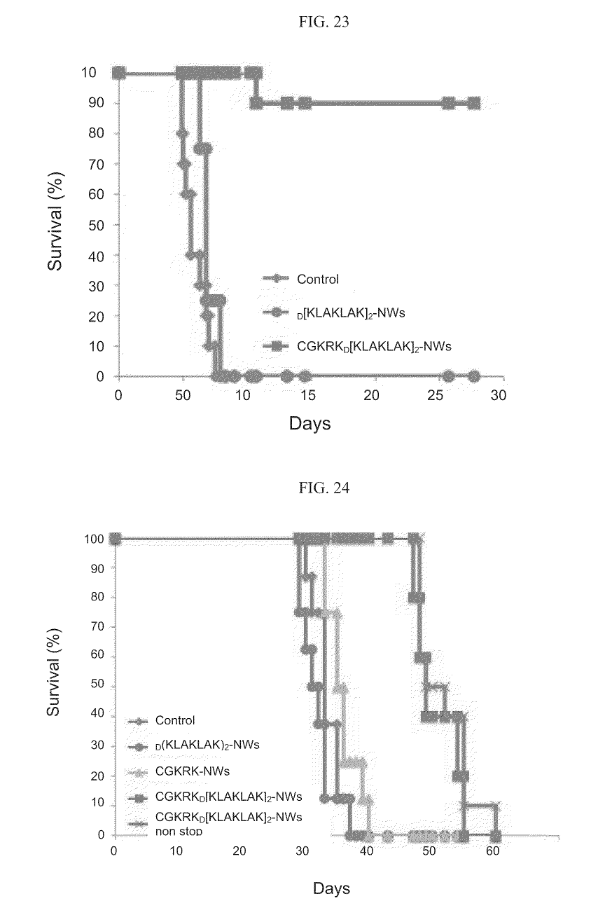

Given the observations that the targeted pro-apoptotic peptide-coated NWs are more effective in cell death than the peptide alone and that the cell death is induced by the same mechanism, the therapeutic effect of CGKRK-_{D}[KLAKLAK]₂-NWs in GBM was tested. Mouse glioblastoma models were used that closely resemble human glioblastomas in their aggressiveness and the diffuse spreading of the tumor cells into the normal brain tissue (Marumoto, 2009). The model was further refined by using a single lentiviral vector expressing H-RasVl2 oncogene and a siRNA targeting p53 (Soda, 2011). Mice injected with the lentivirus in the hippocampus invariably develop glioblastomas that have a highly predictable course of tumorigenesis that results in the death of the mice 2 to 3 months post-injection. Systemic CGKRK-_{D}[KLAKLAK]₂-NWs treatment for 3 weeks every other day cured almost all mice injected with the H-RasVl2-sip53 lentiviral vector compared with mice that received PBS or _{D}(KLAKLAK)₂-NWs alone, that succumbed to the disease almost at the same time (FIG. 23). Luciferase signal was monitored (the lentiviral vector contains the luciferase reporter) starting from 6 weeks post-viral injection (that also correlates to the end of the treatment) and at 6, 7.5, 8, 9.5, 11, and 13 weeks. Tumor was not visualized in the mice treated with CGKRK-_{D}[KLAKLAK]₂-NWs. In addition, H&E staining showed a lack of detectable tumor tissue in a mouse treated with CGKRK-D[KLAKLAK]₂-NWs compared to a control mouse that has a relative big tumor at the end of the treatment. Histological analysis of the CGKRK-_{D}[KLAKLAK]₂-NW treated tumors at the end of the study showed small tumors with a lot of particles in the blood vessels compared to non targeted _{D}[KLAKLAK]₂-NW that revealed big tumors and no evidence of particles in the tumor.

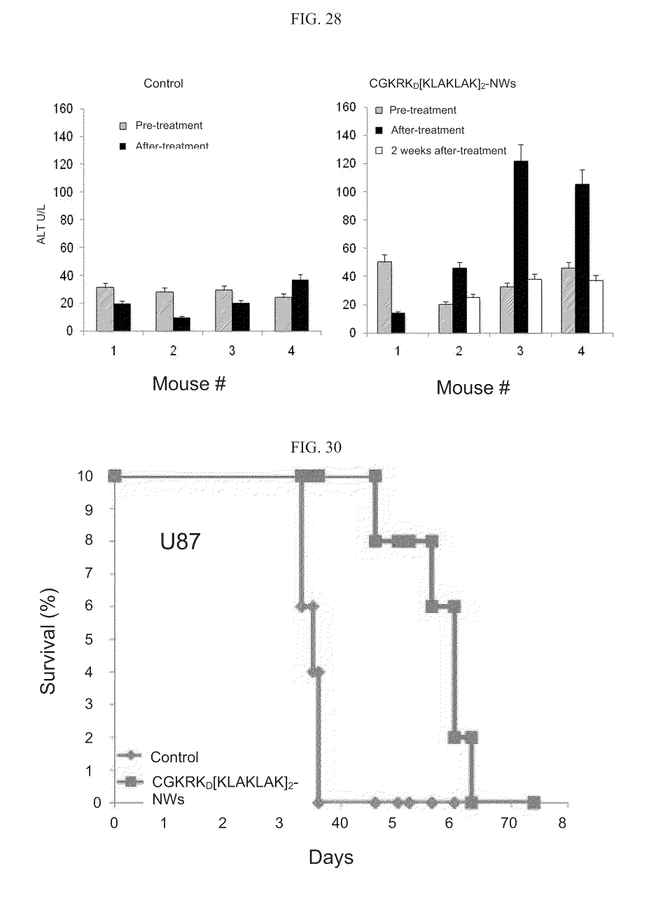

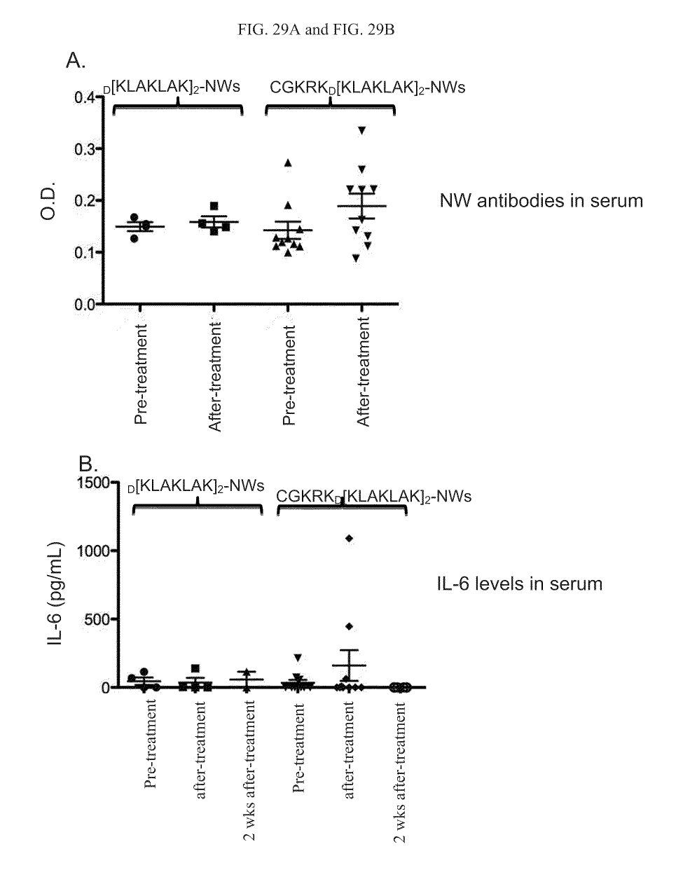

Toxicology analyses indicated some liver toxicity (nanoparticles non-specifically accumulate in the liver) judging from a moderate elevation in the serum level of the liver enzyme L-alanine-2-oxoglutarate aminotransferase (FIG. 28). The values normalized within 2 weeks after the treatment was discontinued. The particles are not immunogenic as was determined by ELISA against anti-Rhodamine Abs in serum of treated mice (FIG. 29A). Minor evidence was found of macrophage activation in an IL-6 assay (FIG. 29B). Furthermore, there is no evidence of damage to the kidney by H&E staining These modest toxicities are in contrast with the severe toxicity of the monovalent _{D}[KLAKLAK]₂ conjugates that have been used before (e.g., Arap, 2002).

The second model uses a tumor cell line (005), which was originally isolated from a glioblastoma tumor induced by the lentiviral method (Marumoto, 2009). Mice transplanted with 005 tumor cells usually die 5-6 weeks post inoculation. The tumors retain the invasive human glioblastoma-like properties described before (Marumoto, 2009). CGKRK-_{D}[KLAKLAK]₂-NW treatment increased the median survival time from 32 to 52 days in this experiment (FIG. 24). Continuous treatment given in a repeated experiment with this model did not give further benefit (FIG. 24). Confocal microscopy at the end of the treatment confirmed that many of the blood vessels in the tumors of the mice treated with CGKRK-_{D}(KLAKLAK)₂-NWs were filled with peptide particles. Lectin perfusion of the 005 tumor mice at the end of the treatment showed that the treated tumors have almost no blood vessel perfusion, indicating that the targeted NWs have destroyed the vast majority of the blood vessels. In U87, a human glioblastoma cell line, the development of the tumor was also significantly delayed (30 days) (FIG. 30).

vi. Enhancement of the Tumor Penetration and Therapeutic Efficacy of CGKRK_{D}(KLAKLAK)₂-NW by iRGD.

A peptide dubbed iRGD [sequence: CRGD(K/R)GP(D/E)C (SEQ ID NO:4); CendR sequence underlined] enhances tumor penetration of iRGD-bound and, surprisingly, co-administered compounds (Sugahara, 2009; Sugahara, 2010; U.S. Patent Application Publication No. 2009-0246133). This peptide comprises two active sites: an RGD motif (Ruoslahti, 2002) and a cryptic CendR sequence RGDK (Teesalu, 2009). The RGD homing motif directs the peptide to av integrins on tumor endothelium, where the peptide is proteolytically processed to expose the CendR motif at the C-terminus. The activated CendR motif binds to neuropilin-1 (NRP-1), which mediates extravasation, tumor penetration, and cell entry of the C-terminally truncated peptide (Sugahara, 2009; Teesalu, 2009). Co-administration of iRGD with uncoupled drugs increases the accumulation and spreading of the drug in tumor tissue, enhancing the activity of the drug, but not the side effects (Sugahara, 2010).

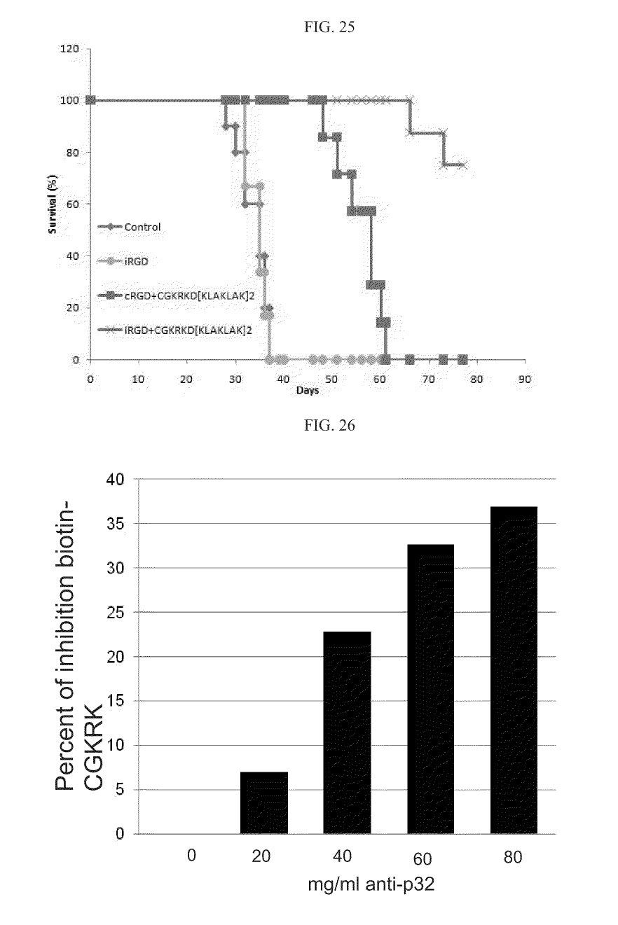

The combination of the NWs with iRGD in a 005 tumor model (NW treatment was only partially successful) was tested in order to determine that iRGD co-administration system can be used to enhance the tumor penetration and therapeutic efficacy of CGKRK-D[KLAKLAK]₂-NR. Non-labeled iRGD was intravenously co-injected with CGKRK-D[KLAKLAK]₂-NW and confocal microscopy analysis showed that NWs co-injected with iRGD were able to spread into the extravascular tumor tissue compared to co-injecting with CRGDC where the particle accumulated mainly in tumor vessels. CGKRK-D[KLAKLAK]₂-NWs co-injected with iRGD treatment increased the median survival time from about 50 days to greater than 80 days (FIG. 25).

2. Discussion

The peptide used in this example was shown to have specific affinity for mitochondria. Several lines of evidence show that CGKRK has the ability to take a payload to the mitochondria. First, live cell imaging shows colocalization with the mitochondria marker than phage binding assay and inhibition assay to purified mitochondria from mouse liver show the specificity of CGKRK to mitochondria. Second, pull-down assays of mitochondria extracts with CGKRK peptide revealed a specific band identified by mass spectrometry as p32. The p32 protein, a homotrimer in solution and solid state, is primarily mitochondrial, but it can be found in the cytoplasm, nuclei, and at the cell surface (Ghebrehiwet, 1994; Braun, 2000; Dedio, 1996; Kittlesen, 2000; Mandi, 2002; Mandi, 2001). The tumor homing peptide Lyp-1 targets also p32 (Fogal, 2008). CGKRK and Lyp-1 (CGNKRTRGC; SEQ ID NO:127) could share the same binding site on p32. The affinity binding of CGKRK is 15 time higher then Lyp-1 (Fogal, 2008), which may be attributable to its size and linear structure which due to the decreased steric cloud at each of the three binding sites of the trimeric protein.

Many reports previously describe the conjugation of the pro-apototic peptide, _{D}[KLAKLAK]₂, to different cell penetrating peptide (Arap, 2002; Fantin, 2005; Karjalainen, 2011; Mai, 2001; Rege, 2007), antibody fragment (Marks, 2005; Rege, 2007), or encapsulation into nanostructures (Ko, 2009; Standley, 2010) to target special types of tumor. Other sequence modifications introducing more hydrophobic residues lead to increase internalization and the toxicity (Horton, 2009). The main limitation of this treatment is the high dose of _{D}[KLAKLAK]₂ needed causes kidney toxicity presumably by the non-proteolysable D residues in the peptide (Arap, 2002; Karjalainen, 2011). In this study the CGKRK-_{D}[KLAKLAK]₂ peptide was coupled to NWs, which creates a multifunctional display of the peptide and makes the peptide hundreds of times more effective than the monomeric peptide (NWs LC₅₀ value was 0.05-0.15 μM compare with the free peptide 14-25 μM) (Table 3). An untargeted multivalent display of the D(KLAKLAK)₂ on nanoparticles was reported to enhance in vitro internalization of the nanoparticles into cells (Standley, 2010) while the in vivo effects were not studied. Furthermore, results from histopathology and blood toxicity assays after tumor treatment indicated that the NWs do not elicit any apparent toxicity or negative health effects.

This example shows that CGKRK-_{D}[KLAKLAK]₂-NWs induce cell death by apoptosis in the same mechanism as the peptide conjugate _{D}[KLAKLAK]₂ alone (Ellerby, 1999). Consistent with the cell viability results, CGKRK-_{D}[KLAKLAK]₂-NWs and D[KLAKLAK]₂-NWs induced Annexin expression whereas CGKRK-NWs did not (FIG. 21). Washing the cells a short time after the particle incubation highlights the targeting efficacy of the moiety in this chimera. The cell death by CGKRK-_{D}[KLAKLAK]₂-NWs was caspase dependent obtained through pro-caspase 3 processing. Apoptosis in proliferating endothelial cells, cancer endothelial cells and GBM cell line in vitro cells treated with the CGKRK-_{D}[KLAKLAK]₂-NWs indicate that CGKRK mediated delivery into the cells and subsequent internalization into mitochondria then _{D}[KLAKLAK]₂ motif disturbs mitochondrial membrane to cause cell death.

CGKRK-NWs home to blood vessel in prostate cancer (Agemy, 2010). Glioblastomas are generally highly angiogenic tumors (Chi, 2009; Jain, 2007) thereby serves as an ideal model for this therapy. In the current study it was found that CGKRK peptide shows significant homing to GBM tumor models and CGKRK-NWs home to GBM tumor blood vessels. Conjugation of CGKRK-_{D}[KLAKLAK]₂ to iron oxide retained its biological properties in different types of GBM including a human model. This example shows that CGKRK-_{D}[KLAKLAK]₂-NWs are a good contrast agent for brain glioma and could be used to selectively improve the detection of tumor with MRI in vivo.

3. Experimental Procedures

Cell lines and tumors. Human umbilical vein endothelial cells (HUVEC; Lonza Walkersville, Walkersville, Md.) were cultured using EBM-2 medium with endothelial cell growth supplement (Lonza Walkersville, Walkersville, Md.). Human astrocytoma cell line (U87) was grown in Dulbecco's modified Eagle's medium (DMEM) plus 10% fetal bovine serum and 1% glutamine pen-strep (Invitrogen, Auckland, NZ). Mouse GBM-initiating 005 cell line was established as described (Marumoto, 2009). The 005 cells were maintained in N2 medium, which contains DMEM/F-12 (Omega Scientific), 1% N2 supplement (Invitrogen), 20 ng/mL human FGF-2 (Prepotech), 20 ng/mL human EGF (Promega, Medison, Wis.), and 40 μg/mL heparin (Sigma-Aldrich, St. Louis, Mo.). T3 cells were obtained by differentiation induction of 005 cells cultured in EGM-2 (Lonza Walkersville, Walkersville, Md.). Human GBM spheres were obtained and cultured as described previously (Soda, 2011). Mouse GBM-initiating 005 cells were transplanted into brains of NOD-SCID mice. A total of 3×10⁵ cells were suspended in 1.5 μl of PBS and injected stereotaxically in the right hippocampus. Human GBM spheres xenografts were created by injecting 0.5×10⁶ cells orthotopically into NOD-SCID mice in 1.5 μl of PBS. Animal experimentation was performed according to procedures approved by the Animal Research Committee at the University of California, Santa Barbara, The Sanford-Burnham Medical Research Institute, and The Salk Institute for Biological Research, San Diego.

Peptide synthesis. Peptides were synthesized with an automatic microwave assisted peptide synthesizer (Liberty; CEM, Matthews, N.C.) using standard solid-phase Fmoc/t-Bu chemistry with 2-(1H-7-azabenzotriazol-1-yl)-1,1,3,3-tetramethyl uronium hexafluorophosphate methanaminium (Anaspec, Inc., San Jose, Calif.) as the coupling reagent. During synthesis, the peptides were labeled with 5(6)-carboxyfluorescein (FAM) (Sigma-Aldrich, St. Louis, Mo.) with a 6-aminohexanoic acid spacer separating the dye from the sequence. The peptides were cleaved from the resin using 95% trifluoroacetic acid (Sigma-Aldrich, St. Louis, Mo.) with 2.5% water and tri-isiopropylsilane (Sigma-Aldrich, St. Louis, Mo.). Subsequent purification by High Performance Liquid Chromatography (Gilson Inc., Middleton, Wis.) gave peptides with >90% purity.

Isolation of mitochondria and peptide/phage binding. Mitochondria were isolated from livers of Balb/c mice using differential centrifugation with buffers from a Pierce mitochondrial isolation kit for tissue according to the manufacture's instruction (Pierce Biotechnology, Rockford, Ill.). To test the binding of the CGKRK peptide to mitochondria, purified mitochondria were pre-incubated with various concentrations of non-labeled CGKRK or control peptide (CREKA; SEQ ID NO:92) for 30 min 4° C. FAM-CGKRK was then added and incubated for an additional 1 hour. The binding of the FAM peptide was quantified by fluorescence. In phage binding assays, purified mitochondria were suspended in 10 ml DMEM supplemented with 1% BSA, and incubated with 5×10⁸ plaque-forming units (pfu) of peptide-displaying phage, overnight at 4° C. The mitochondria were washed 3 times with DMEM/BSA, the phage were collected with lysogeny broth containing 1% NP-40, and quantified by plaque assay.

Affinity chromatography. Mitochondria were lysed in PBS containing 400 mM n-octyl-beta-D-glucopyranoside (Calbiochem, La Jolla, Calif.), and clarified lysates were incubated with CGKRK-coated Sulfolink-beads (Pierce biotechnology, Rockford, Ill.). After washing, bound proteins were eluted with lysis buffer containing 2 mM free CGKRK peptide and separated by SDS-PAGE. Gel bands excised from silver-stained gels were analyzed by MALDI-TOF mass spectrometry at the Burnham Institute for Medical Research Proteomics Resource.

Affinity measurements. The affinity of CGKRK for p32 was measured by an ELISA-based assay. Wells in 96-well plates were coated with 3 μg/ml of purified p32 protein and incubated for 1 hour at 37° C. with various concentrations of biotinylated LyP-1 peptide in PBS (100 μl/well). After washing with TBS containing 1 mmol/l CaCl₂ and 0.01% Tween 20, streptavidin-conjugated horseradish peroxidase (Zymed, San Francisco, Calif.) was added to the wells and incubated for 1 hour at room temperature. Peptide binding to p32 was quantified with 2,2-azino-bis(3-ethylbenzthiazoline-6-sulfonic acid) (Sigma-Aldrich, St. Louis, Mo.) as substrate. Wells without p32 coating were used to determine background binding. Kd values were calculated using Prism software.

Cell proliferation assay and imaging. The MTT [344,5-dimethylthazol-2-yl]-2,5-diphenyl tetrazolium-bromide] assay (Molecular Probes, Eugene, Oreg.) was used to quantify cell proliferation. Cells were seeded in complete medium into 96-well plates (5×10³ cells/well), and allowed to attach overnight at 37° C. in a humidified 5% CO₂ atmosphere. The culture media was then removed and various concentrations of peptides or NWs were added. After 48, and 72 hours, 10 μL of the MTT reagent (5 mg/mL in PBS) was added to each well. The medium was removed from cells after 3 hours, and 100 ml of DMSO:MeOH (1:1 v/v) were added to each well. The plates were read at a wavelength of 595 nM. For live cell imaging, cells seeded on glass-bottom plates (Willco, Amsterdam, Netherlands), and 24 hours later, the cells were washed and incubated with FAM-labeled peptides or NWs for 45 min at 37° C. The cells were then rinsed three times with PBS and incubated for an additional 15 min at 37° C. with 500 nm MitoTracker Red (Molecular Probes, Eugene, Oreg.) followed by nuclear staining with Hoechst 33342 DNA dyes (Molecular Probe, Eugene, Oreg.) for 12 min. The cells were analyzed with Fluoview FV 500 confocal microscopy (Olympus America, Center Valley, Pa.).

Immunoblot analysis of NW-bound proteins. HUVEC cells were incubated 24 and 48 hr with 10 μg/ml CGKRK_{D}[KLAKLAK]₂-NWs or CREKA-NW and lysed with RIPA buffer (Pierce, Rockford, Ill.) according to the manufacturer's instructions. The lysates were separated by SDS-PAGE. After transfer of the proteins onto nitrocellulose membranes for 2 hours at 200 mA, the membrane was treated for 1 hour at room temperature with TBS-0.05% Tween containing 5% milk, incubated with 1 mg/ml anti-caspase 3 (Cell Signaling, Denvers, Mass.) and anti-p32 (R&D system, Minneapolis, Minn.), followed by anti-rabbit IgG, HRP-linked antibody (Cell Signaling, Denvers, Mass.).

Flow cytometry. Cells were harvested and stained using the Annexin V-PE apoptosis detection kit (BD Pharmingen) and analyzed on a BD LSR II flow cytometer (Becton Dickinson).

In vivo matrigel angiogenesis. Two-month old Balb/c nu/nu mice were subcutaneously injected bilaterally in the inguinal area with 500 ml of matrigel (Becton Dickson, Bedford, Mass.) with or without of 500 ng recombinant human bFGF (Becton Dickson, Bedford, Mass.) as an angiogenesis stimulant. The mice were treated every other day with PBS or CGKRK-_{D}[KLAKLAK]₂-NWs (5 mg/kg) for 2 weeks. At the end of the treatment, the mice were sacrificed under anesthesia by perfusion through the heart with far-red fluorescent Alexa Fluor 647 isolectin GS-IB4 conjugate (Molecular Probes, Eugene, Oreg.), followed by further perfusion with ˜10 ml of 4% PFA. The matrigel plugs were imaged by the Odyssey Infrared Imaging System (LI-COR Biotechnology, Lincoln, Nebr.), and for histological analyses, 7-10 μm sections were cut and viewed under a Fluoview 500 confocal microscope (Olympus America, Center Valley, Pa.). The treatment and PBS control groups consisted of two mice (four plugs) in each of three independent experiments.

Tube formation assay. Wells in 24-well plates were coated with 250 μl matrigel (Becton Dickison, Bedford, Mass.). HUVECs were detached with trypsin (Lonza Walkersville), washed with PBS, and 10⁵ cells in EBM2 media with supplement as mentioned above were subsequently plated on top of the matrigel in the presence of 5 or 10 μg/ml of NWs. The cells were incubated at 37° C. for 24 hours and photographed under a bright field microscope at 40× magnification (Leica Microsystems, Wetzlar, Germany).

In vivo peptide homing. Mice with orthotopic brain cancers induced as described above were used when they showed symptoms of the presence of a tumor mass. Rhodamine-labeled CGKRK (200 μg) was intravenously injected into the mice and allowed to circulate for 3 hours. The mice were perfused with PBS through the heart under anesthesia, and tissues were collected and processed for fluorescence analysis.

Preparation of Nws. Nws Coated with Peptides were Prepared as Described (Agemy, 2010; Park, 2009). Aminated nanoworms were pegylated with maleimide-5KPEG-NHS (JenkemTechnology, City, China). The aminated nanoworms were pegylated with maleimide-5 KPEG-NHS (JenkemTechniology, China). Peptides were conjugated to the nanoparticles through a thioether bond between the cysteine thiol from the peptide sequence and the maleimide on the functionalized particles.

Quantification peptide on NWs. The aminated nanoworms were pegylated with OPSS-5 KPEG-NHS (JenkemTechniology, China). Peptides were conjugated to the nanoparticles through a disulfide bond between the cysteine thiol from the peptide sequence and the pyridyl sulfenyl protected thiol on the functionalized particles. The CGKRK-_{D}[KLAKLAK]₂-NWs linked covalently through the disulfide linkage were treated with DTT. The concentration of free peptide thus obtained in the solution was estimated using fluorescence spectroscopy with a peptide standard curve.

In vivo NW injections. Mice bearing orthotopic GBM tumors were injected into the tail vein with NWs (5 mg of iron per kg body weight). In homing experiments, the mice were euthanized 5-6 hours after the injection by cardiac perfusion with PBS under anesthesia, and organs were dissected and analyzed for NWs. In tumor treatment experiments, tumor mice were intravenously injected with NWs in 150 μl PBS, or PBS as a control every other day for 3 weeks. Mice with 005 tumors were also intravenously injected with CGKRK-_{D}[KLAKLAK]₂-NW (5 mg of iron/kg) in combination with 4 mmol/kg of either the tumor-penetrating peptide, iRGD, or CRGDC (SEQ ID NO:136) as a control. At the end of the treatment, 2 mice per group were euthanized and the rest of the mice were monitored until the animal facility staff determined that a mouse's symptoms required euthanasia.

Histology and immunohistology. Tissues were fixed in 4% paraformaldehyde overnight at 4° C., cryo-protected in 30% sucrose overnight and frozen in OCT embedding medium. Tissue sections (7 μm) were cut and H&E stained or processed for immunostaining To stain for CD31, sections were first incubated for 1 hour at room temperature with 10% serum from the species in which the secondary antibody was generated, followed by incubation with monoclonal anti-mouse CD31 (10 mg/ml; BD Pharmingen, San Jose, Calif.), and Alexa 647 goat anti-rat secondary antibody (1:1000; Molecular Probes, Eugene, Oreg.). Each staining experiment included sections stained with the secondary antibody only as a negative control. Nuclei were counterstained with DAPI (5 mg/mL; Molecular Probes). The sections were mounted in Gel/Mount mounting medium (Biomeda, Foster City, Calif.) and viewed under a Fluoview 500 confocal microscope (Olympus America, Center Valley, Pa.).

Biophotonic tumor imaging. Mice bearing luciferase-labeled GBM tumors received injections of 3 mg per mouse of freshly prepared luciferin substrate (Promega, Medison, Wis.) suspended in PBS. The mice were then anesthetized with isofluorane and imaged using the Xenogen IVIS® 100 Imaging System (Xenogen, Caliper LifeSciences), 10 minutes post intraperitoneal injection of luciferin at a 1-minute acquisition time in a small binning mode.

NW toxicity studies. Serum was collected from mice before treatment, one day after the treatment ended, and two weeks after the treatment was concluded. ALT levels in serum were determined using ALT (GPT) Reagent (Infinity™) following the manufacturer's instructions. The same serum samples were used to determine the levels of IL-6 using the Mouse IL-6 ELISA Set (BD OptEIA™, BD Biosciences).

Magnetic resonance imaging. Mice bearing orthotopic 005 tumors were intravenously injected with CGKRK-_{D}[KLAKLAK]₂-NW (5 mg of iron/kg). Approximately 5 hours after the NW injection, the mice were anesthetized with isoflurane and subjected to T2* weighted MRI using the following conditions: 3D spoiled gradient echo, TR=40 ms, TE=18.6 ms, Flip angle=15 deg, bandwidth=115 Hz/pixel, resolution: (0.16 mm)₂ in plane and 0.3 mm slice thickness). Three slices averaged for improved signal-to-noise). The instrument was Sigma HDx 3 T scanner (GE Healthcare, Milwaukee, Wis.). After imaging, tissues of interest were harvested and processed for H&E staining.

Statistical analysis. Data were analyzed by two-tailed Student's unpaired t-test. P values of less than 0.05 were considered statistically significant.

Glioblastomas are generally highly angiogenic tumors, and VEGF is produced in high levels by the tumor cells (Chi, 2009; Jain, 2007). Therefore, anti-angiogenic therapy was thought to be a promising therapeutic strategy particular for glioma (Jain, 2007). The best known antiangiogenic agents are inhibitors of VEGF-A, notably, bevacizumab, a neutralizing antibody to VEGF-A, demonstrating good anti-glioma activity in preclinical study, but only marginal effect in the clinic. Combining angiogenesis inhibitors with other anti cancer agents showed better therapeutic effect. For example, in a phase II clinical trial, more than half of the patients with GBM responded to the combination treatment of anti-VEGF antibody bevacizumab and irinotecan, but this effect was transient in most patients (Vredenburgh, 2007). Mechanisms proposed to explain resistance to anti-VEGF therapy include activation of other proangiogenic signaling pathways, recruitment of bone marrow (BM)-derived myeloid cells that protect and nurture vascular cells, protection of blood vessels by increased pericyte coverage, and increased tumor invasion (Bergers, 2008; Shojaei, 2008). Recent studies have shown that lack of VEGF-A receptor (VEGFR2) and bFGF receptor by transdifferentiation of glioblastoma cells into endothelial cells are potentially the main contribution for this resistance (Soda, 2011).

4. Results

i. Homing of CGKRK Peptide to Glioblastoma (GMB) Tumors and Interaction of CGKRK Peptide with Mitochondria.

Mice bearing 005 glioma tumors in the right hippocampus were intravenously injected with 200 μg of CGKRK peptide labeled with rhodamine. After 3 hours, the mice were perfused through the heart with PBS, and the tumor and normal brain tissue were collected. The inset in last panel shows section of normal brain tissue. CGKRK peptides congregate in tumor blood vessels. Proliferating human endothelial cells (human umbilical vein endothelial cells (HUVEC); resembling angiogenic endothelial cells) and U87 cells were incubated with FAM-CGKRK peptide and MitoTracker and examined by fluorescent microscopy to assess whether the CGKRK peptide targets mitochondria. The coincidence of the two labels showed that CGKRK peptides co-localized with mitochondria. FAM-CGKRK was incubated with purified mitochondria in the presence of increasing concentrations of either unlabeled CGKRK or an unrelated peptide (CREKA) as a control (FIG. 17). Binding of the labeled CGKRK peptide declined in proportion to the amount of unlabeled CGKRK peptide but not in the case of the CREKA peptide, indicating that the binding to mitochondria by the CGKRK peptide is specific. CGKRK phage and CREKA phage (as a control) were incubated with purified mitochondria (FIG. 18). Titration of bound phage shows about 80 times more binding of the CGKRK phage than the control.

ii. CGKRK Peptide Binds to p32 Protein in Mitochondrial Extracts.

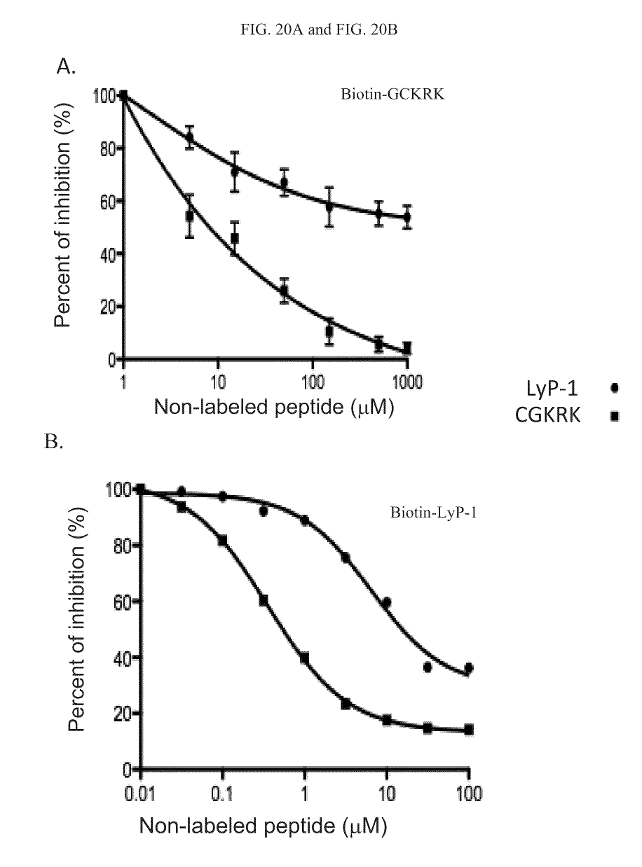

Proteins were extracted from mitochondria purified from mouse livers and fractionated by affinity chromatography on CGKRK peptide coupled SulfoLink Resin. Bound proteins were eluted with free CGKRK peptide (2 mM), or CREKA peptide as a control. p32 protein eluted rapidly from the affinity matrix with the CGKRK peptide but did not elute with the control CREKA peptide. This indicates that the CGKRK peptide binds specifically to the p32 protein. An anti-p32 immunoblot of elution samples was performed. This confirmed that the eluted protein corresponds to the p32 protein. Binding of increasing amounts of biotin-labeled CGKRK peptide to immobilized p32 protein was detected with streptavidin coupled to horseradish peroxidase and normalized to nonspecific binding in the absence of p32 (FIG. 19). The affinity of the peptide for p32 calculated from the binding curves is Kd=0.2±0.068 μg/ml. Percent of inhibition was assessed using binding inhibition curves in the presence of increasing concentrations of non-labeled CGKRK peptide or LyP-1 peptide (FIG. 20). Inhibition of binding drops much more quickly for both peptides in the presence of non-labeled CGKRK than for non-labeled LyP-1 peptide.

iii. Homing of CGKRK_{D}[KLAKLAK]₂-nanoworms (NWs) to GMB Tumors.

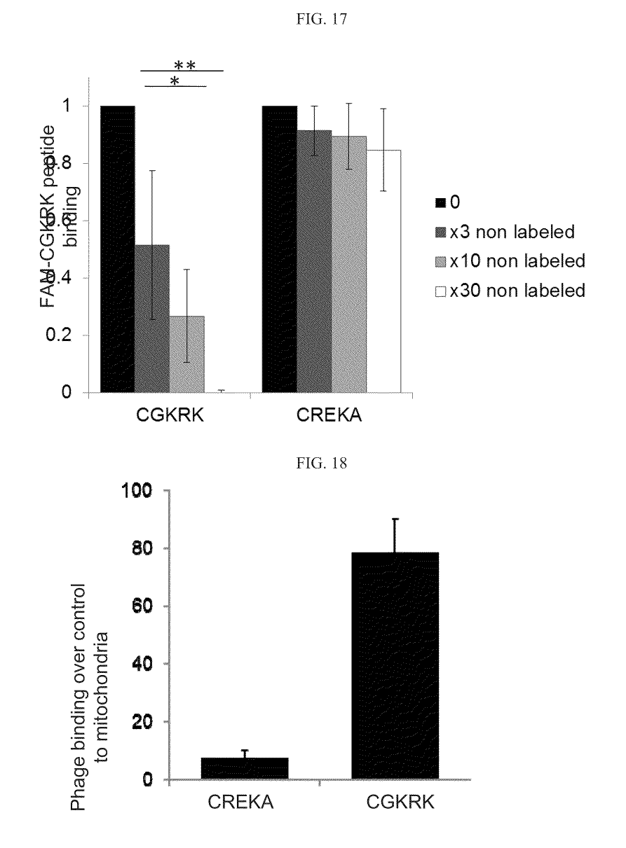

A chimeric peptide consisting of a tumor-homing peptide (CGKRK) and a pro-apoptotic peptide (_{D}[KLAKLAK]₂) was covalently coupled to iron oxide nanoparticles (NWs; length 80-100 nm, width 30 nm; FIG. 16). Iron oxide NWs coated with Rd-labeled CGKRK-_{D}[KLAKLAK]₂ peptide (through a 5K-PEG linker) were intravenously injected (5 mg iron per kg body weight) into mice bearing either 005 tumors, or xenograft tumors generated with human GBM spheres or U87 cells. The tumor cells were injected into the right hippocampus. Five to six hours after the injection, the mice were perfused through the heart with PBS, and the organs were collected. Tumor sections were stained and examined by confocal microscopy. The Rd-labeled CGKRK-_{D}[KLAKLAK]₂-coated particles, tumor cells (both the human GBM spheres and U87 cells expressed green fluorescent protein), blood vessels stained with anti-CD31, and nuclei stained with DAPI were visualized. CGKRK peptides congregate in tumor blood vessels for all three tumor types. Rd-labeled CGKRK_{D}[KLAKLAK]₂-NWs were intravenously injected into tumor-bearing mice and tumors visualized by T2* weighted MRI (3D spoiled gradient echo. Signal from the nanoworm compositions coincided with the tumor location.

iv. _{D}[KLAKLAK]₂CGKRK-NW Conjugates Internalize into Activated Endothelial Cells, co-localize with Mitochondria, and Induce Cell Death by Apoptosis.

Live HUVEC were incubated for 2 hours at 37° C. in the presence of fluorescein (FAM)-labeled NWs (CGKRK peptide, _{D}[KLAKLAK]₂ peptide, or CGKRK_{D}[KLAKLAK]₂ peptide and for 15 minutes in the presence of a marker for mitochondria (MitoTracker). DNA was counterstained with Hoechst 33342. The cells were visualized by confocal microscopy. Only the CGKRK_{D}[KLAKLAK]₂ peptide showed extensive localization in mitochondria. FAM-CGKRK_{D}[KLAKLAK]₂ peptide was coupled onto the NWs via a reducible 5-kDa PEG linker. The linker was cleaved from the NWs using DTT, and the amount of peptide present on the NWs was determined by fluorescence measurements in solution (to circumvent quenching on the NW surface) and used to calculate IC₅₀ (Table 3).

TABLE 3

IC50

| Cell Line | Peptide (μM) | Peptide on NW | Fold Increase |
|---|---|---|---|
| HUVEC | 14 | 0.05 | 280.0 |
| T3 | 25 | 0.15 | 166.7 |
| U87 | 16 | 0.15 | 106.7 |

HUVEC and T3 cells were left untreated (Control) or treated with a concentration of 10 μg/ml of NWs coated with either a control peptide (CREKA), _{D}[KLAKLAK]₂, or CGKRK_{D}[KLAKLAK]₂ for 24, 48 and 72 hours (FIG. 21) or the particles were washed away after 30 min and the incubation was continued for 72 hrs (FIG. 22). The cells were stained with Annexin and analyzed by flow cytometry. The total percentage of Annexin-positive cells (apoptotic and dead cells) is indicated in FIGS. 21 and 22. Whole cell extracts (HUVEC) from the experiment in FIG. 21 were prepared and analyzed by immunoblotting using antibodies against cleaved caspase-3, and β-actin as loading control and also by confocal microscopy with cleaved caspase-3, tubulin, and nuclei stained. Caspase-3 showed cleavage only in the cells incubated with CGKRK_{D}[KLAKLAK]₂ nanoworms.

v. CGKRK_{D}[KLAKLAK]₂-NW Treatment of Tumors Induced by Lentiviral Injection.

Mice bearing lenti-viral (H-RasV12-sip53) induced brain tumors in the right hippocampus were intravenously injected with NW coated with peptides. The particles were administered every other day for 18 days, starting 3 weeks post-viral injection. FIG. 23 shows the survival curves of the non-treated (control) mice and for mice treated with _{D}[KLAKLAK]₂ nanoworms or CGKRK_{D}[KLAKLAK]₂ nanoworms. CGKRK_{D}[KLAKLAK]₂ nanoworms provide a dramatic increase in survival compared to the control and the _{D}[KLAKLAK]₂ nanoworms. The mice were monitored for luciferase signal using the IVIS system (the lentiviral vector contains the luciferase reporter). Only the control mice had detectable luciferase signal. After H&E staining at the end of the treatment, cancer was visible at the lentiviral injection site only in control mice. Confocal microscopy images of brain sections from a representative mice at the end of the treatment showed a small residual tumor in the with CGKRK _{D}[KLAKLAK]₂-NW-treated mouse but significantly more tumor cells in the _{D}[KLAKLAK]₂-NW-treated mouse.

vi. Treatment of Transplanted GBM Tumors with CGKR1(_{D}[KLAKLAK]₂-NWs.

Tumors were developed by transplanting 3×10⁵ 005 cells into the right hippocampus of NOD-SCID mice. Ten days post-tumor cell transplantation, the mice were intravenously injected with NWs. The NWs (5 mg of iron/kg) were administered every other day for 3 weeks or administered non-stop for the same period of time (n=8 per group). A, FIG. 24 shows survival curves of mice treated with _{D}[KLAKLAK]₂-NWs, CGKRK-NWs, and CGKRK_{D}[KLAKLAK]₂-NWs. CGKRK_{D}[KLAKLAK]₂-NWs exhibited longer survival whether administered every other day or non-stop. Brain sections of representative mice at the end of the treatment were stained with anti-CD31 and DAPI. The CGKRK _{D}[KLAKLAK]₂-NWs and _{D}[KLAKLAK]₂-NWs were labeled with rhodamine. The tumor cells expressed green fluorescent protein. CGKRK _{D}[KLAKLAK]₂-NWs homed to tumor blood vessels. _{D}[KLAKLAK]₂-NWs did not home to tumors. Lectin was perfused into representative tumor mice at the end of the treatment. Vessels were stained by perfusion of biotinylated Lycopersicon esculentum lectin and visualized by confocal microscopy using anti-biotin. Mice treated with CGKRK _{D}[KLAKLAK]₂-NWs showed little tumor vessel labeling and less tumor cell labeling compared to the control mice indicating reduction in tumors and tumor vessels.

vii. Enhanced Anti-tumor Effect of CGKRK _{D} [KLAKLAK]₂-NWs co-injected with iRGD.

Mice bearing orthotopic 005 tumors were intravenously injected with CGKRK _{D}[KLAKLAK]₂-NW (5 mg of iron/kg) in combination with 4 mmol/kg of either non-labeled CRGDC or iRGD peptide. The tumors and tissues were collected 5-6 hours later, and analyzed by confocal microscopy. CGKRK _{D}[KLAKLAK]₂-NWs in mice coinjected with CRGDC mostly coincided with tumor blood vessels, indicating homing to tumor blood vessels. CGKRK _{D}[KLAKLAK]₂-NWs in mice coinjected with iRGD were localized within the tumor away form the tumor blood vessels indicating increase cell internalization and tissue penetration. Mice bearing orthotopic 005 tumors implanted 10 days earlier received every other day for 3 weeks intravenous injections of either CGKRK _{D}[KLAKLAK]₂-NWs (5 mg of iron/kg) mixed with 4 mmol/kg of cRGD or iRGD. FIG. 25 shows survival curves are shown (n=8-10 per group). Mice treated with CGKRK _{D}[KLAKLAK]₂-NWs showed a clear increase in survival time compared to control mice and mice treated with iRGD alone, with mice treated with CGKRK _{D}[KLAKLAK]₂-NWs and iRGD together showing the longest survival times (80% of the mice still alive after 80 days).

viii. CGKRK Peptide and CGKRK _{D}[KLAKLAK]₂-NWs in Normal Tissue of Tumor-bearing Mice.

Mice bearing 005 tumors in the right hippocampus were intravenously injected with 200 μg of Rd-labeled CGKRK peptide. After 3 hours, the mice were perfused through the heart with PBS, and tissues were collected, sectioned and analyzed for rhodamine fluorescence. Significant CGKRK peptide appeared in kidney but not in heart, pancreas, liver, lung, or spleen. Rd-labeled CGKRK _{D}[KLAKLAK]₂-NWs were intravenously injected (5 mg iron per kg body weight) into mice bearing 005 tumors. The tumors were generated by injecting 005 tumor cells into the right hippocampus. Five to six hours after the injection, the mice were perfused through the heart with PBS, and tissues were collected. Tumor sections were stained with antibodies and examined by confocal microscopy. Significant CGKRK _{D}[KLAKLAK]₂-NWs appeared in kidney but not in pancreas, liver, lung, spleen, or normal brain. The label also appeared in urine of the mice.

ix. Inhibition of CGKRK Peptide Binding to p32 by Anti-p32.

Biotin-CGKRK at 1 μg/ml was incubated in microtiter wells coated with purified p32, and the binding was detected with streptavidin coupled to horseradish peroxidase and normalized to nonspecific binding in the absence of p32. The anti-32 antibody was prepared against the full-length p32 protein (Protein Production and Analysis Facility of the Sanford-Burnham Medical Research Institute). The experiments were performed in triplicate; one of two experiments with similar results is shown in FIG. 26. The result show that the CGKRK peptide binds to p32.

x. Homing of CGKRK-NWs to GMB Tumors.

Iron oxide NWs coated with Rd-labeled CGKRK or _{D}[KLAKLAK]₂ peptide through a 5K-PEG linker were intravenously injected (5 mg iron per kg body weight) into mice bearing 005 tumors. The tumors were generated by injecting 005 tumor cells into the right hippocampus. Five to six hours after the injection, the mice were perfused through the heart with PBS, and tissues were collected. Tumor sections were stained with antibodies and examined by confocal microscopy. The CGKRK-NWs homed to blood vessels in the tumors. The _{D}[KLAKLAK]₂-NWs did not collect in the tumors.

xi. CGKRK_{D}[KLAKLAK]₂-NW conjugates induce cell death by apoptosis.

HUVEC and T3 cells were left untreated (Control) or treated with 10 μg/ml of NWs coated with CGKRK _{D}[KLAKLAK]₂-NWs for 48 or 72 hours. Representative results for these HUVEC and T3 cells indicating the percentage of Annexin-positive cells (apoptotic and dead cells) are shown in FIGS. 27A and 27B, respectively. HUVEC and T3 cells were also incubated with CGKRK-NWs, CREKA-NWs, _{D}[KLAKLAK]₂-NWs, or CGKRK _{D}[KLAKLAK]₂-NWs. The cells were washed after 30 minutes to remove excess NWs and then incubated for 72 hours. Representative results for these HUVEC and T3 cells indicating the percentage of Annexin-positive cells are shown in FIG. 27C. Annexin staining and analysis by flow cytometry were used to measure apoptosis in the cultures.

xii. CGKRK _{D}[KLAKLAK]₂-NW Conjugates Inhibit In Vitro and In Vivo Angiogenesis.

Tube formation assays were performed using primary HUVEC plated on growth factor reduced matrigel in 5% FCS medium alone (Control) or containing CGKRK-NWs or CGKRK _{D}[KLAKLAK]₂-NWs. The formation of networks of capillary-like structures was viewed by phase contrast-microscopy at 40× magnification 24 hours after plating. Capillary formation was disrupted in cells treated with CGKRK _{D}[KLAKLAK]₂-NWs but not in cells treated with CGKRK-NWs. Matrigel plugs with or without bFGF were subcutaneously injected into Balb/c nu/nu mice. The mice were treated every other day with intravenous injections of either CGKRK _{D}[KLAKLAK]₂-NWs (5 mg/kg) or PBS. The mice were euthanized 14 days later, and perfused with 647 Alexa iso-lectin. The matrigel plugs were removed from the mice and viewed macroscopically under fluorescent light or by confocal microscopy analysis of sections of the plugs. Representative mice treated with matrigel and without bFGF and mice treated with matrigel, bFGF, and CGKRK _{D}[KLAKLAK]₂-NWs showed no significant vessel formation while the control (treated with matrigel and bFGF) showed robust vessel formation.

xiii. Toxicology Analyses of Mice Treated with CGKRK_{D}[KLAKLAK]₂-NWs.

Blood L-alanine-2-oxoglutarate aminotransferase (ALT) levels measured before (Pre-treatment), after completion of a 3-week treatment course (After treatment) and after a subsequent 2-week recovery period (2 weeks after treatment) are shown in FIG. 28. A significant increase was seen in two of the mice after treatment with CGKRK _{D}[KLAKLAK]₂-NWs, but this effect had disappeared 2 weeks after treatment. Possible active and innate immune responses against NW was tested by measuring antibody (FIG. 29A) and IL-6 levels (FIG. 29B) in serum of mice collected as described above. The kidneys in control mice and mice treated with CGKRK _{D}[KLAKLAK]₂-NWs were visualized after H&E staining at the end of the treatment. No significant difference was seen between the treatments.

xiv. Treatment of Mice Bearing Intracranial U87 Tumors with CGKRK_{D}[KLAKLAK]₂-NWs.

Tumors were induced by injecting 5×10⁵ GFP-expressing U87 cells into the right hippocampus of mice. Treatment with intravenous injections of CGKRK-_{D}[KLAKLAK]₂-NWs and control NWs was started 10 days after the tumor cell injection and continued every other day for 3 weeks (n=5 per group). Survival curves are shown in FIG. 30. Treatment with CGKRK-_{D}[KLAKLAK]₂-NWs significantly increased the time of survival.

xv. CGKRK-binding Proteins in Glioblastoma Tumor Extracts.

Proteins from brain tumor extracts were bound to insolubilized (SulfoLink Resin; Pierce Biotechnology) CGKRK peptide. Proteins were eluted from the affinity matrix with either CGKRK peptide or with CREKA (SEQ ID NO:92) peptide (negative control). Silver-stained gels were used to show the proteins eluted with CGKRK and CREKA (SEQ ID NO:92). CREKA (SEQ ID NO:92) failed to elute any visible protein bands. CGKRK peptide eluted nardilysin (132 Kd), nucleolin (97 Kd), cytoskeleton-associated protein 4 (p63), Hnrnpa3/Hnrnpa2b1 (39 Kd), and p32 protein (33 Kd). The bands in the CGKRK eluate were identified by mass spectrometry as the proteins listed.

### REFERENCES

Desai N, Trieu V, Yao Z, Louie L, Ci S, Yang A, Tao C, De T, Beals B, Dykes D, Noker P, Yao R, Labao E, Hawkins M, Soon-Shiong P (2006) Clin Cancer Res 12: 1317-1324.
Weissleder R, Bogdanov A, Jr, Neuwelt E A, Papisov M (1995) Advanced Drug Delivery Reviews 16: 321-334.
Sinek J, Frieboes H, Zheng X, Cristini V (2004) Biomed Microdevices 6: 297-309.
Boucher Y, Baxter L T, Jain, R K (1990) Cancer Res 50: 4478-4484.
Hoffman J A, Giraudo E, Singh M, Zhang L, Inoue M, Porkka K, Hanahan D, Ruoslahti E (2003) Cancer Cell 4: 383-391.
Oh P, Li Y, Yu J, Dun E, Krasinska K M, Carver L A, Testa J E, Schnitzer J E (2004) Nature 429: 629-635.
Ruoslahti, E (2002) Nat Rev Cancer 2: 83-90.
DeNardo S J, DeNardo G L, Miers L A, Natarajan A, Foreman A R, Gruettner C, Adamson GN, Ivkov R (2005) Clin Cancer Res 11: 7087s-7092s.
Akerman M E, Chan W C, Laakkonen P, Bhatia S N, Ruoslahti E (2002) Proc Natl Acad Sci USA 99: 12617-12621.
Cai W, Shin D W, Chen K, Gheysens O, Cao Q, Wang S X, Gambhir S S, Chen X (2006) Nano Lett 6: 669-676.
Pasqualini R, Ruoslahti E (1996) Nature 380: 364-366.
Hutchinson J N, Muller W J (2000) Oncogene 19: 6130-6137.
Dvorak H F, Senger D R, Dvorak A M, Harvey V S, McDonagh J (1985) Science 227: 1059-1061.
Abe K, Shoji M, Chen J, Bierhaus A, Danave I, Micko C, Casper K, Dillehay D L, Nawroth P P, Rickles F R (1999) Proc Natl Acad Sci USA 96: 8663-8668.
Pilch J, Brown D M, Komatsu M, Jarvinen T A, Yang M, Peters D, Hoffman R M, Ruoslahti E (2006) Proc Natl Acad Sci USA 103: 2800-2804.
Jung C W, Jacobs P (1995) Magn Reson Imaging 13: 661-674.
Jung, C W (1995) Magn Reson Imaging 13: 675-691.
Weissleder R, Stark, D D, Engelstad B L, Bacon B R, Compton C C, White D L, Jacobs P, Lewis J (1989) AJR Am J Roentgenol 152: 167-173.
Van Rooijen N, Sanders A (1994) J Immunol Methods 174: 83-93.
Moghimi S M, Hunter A C, Murray J C (2001) Pharmacol Rev 53: 283-318.
Moore A, Weissleder R, Bogdanov A, Jr (1997) J Magn Reson Imaging 7: 1140-1145.
Souhami R L, Patel H M, Ryman B E (1981) Biochim Biophys Acta 674: 354-371.
Fernandez-Urrusuno R, Fattal E, Rodrigues J M, Jr, Feger J, Bedossa P, Couvreur P (1996) Biomed Mater Res 31: 401-408.
Radomski A, Jurasz P, Alonso-Escolano D, Drews M, Morandi M, Malinski T, Radomski MW (2005) Br J Pharmacol 146: 882-93.
Khandoga A, Stampfl A, Takenaka S, Schulz H, Radykewicz R, Kreyling W, Krombach F (2004) Circulation 109: 1320-1325.
Van der Heyde H C, Gramaglia I, Sun G, Woods C (2005) Blood 105: 1956-1963.
Gorbet M B, Sefton M V (2004) Biomaterials 25: 5681-5703.
Boccaccio C, Sabatino G, Medico E, Girolami F, Follenzi A, Reato G, Sottile A, Naldini L, Comoglio P M (2005) Nature 434: 396-400.
Huang X, Molema G, King S, Watkins L, Edgington T S, Thorpe P E (1997) Science 275: 547-550.
El-Sheikh A, Borgstrom P, Bhattacharjee G, Belting M, Edgington TS (2005) Cancer Res 65: 11109-11117.
Hutchinson J N, Muller W J (2000) Oncogene 19: 6130-6137.
Laakkonen P, Porkka K, Hoffman J A, Ruoslahti E (2002) Nat Med 8: 751-755.
Laakkonen P, Akerman M E, Biliran H, Yang M, Ferrer F, Karpanen T, Hoffman R M, Ruoslahti E (2004) Proc Natl Acad Sci USA 101: 9381-9386.
Suh T T, Holmback K, Jensen N J, Daugherty C C, Small K, Simon D I, Potter S, Degen J L (1995) Genes Dev 9: 2020-2033.
Van Rooijen N, Sanders A (1994) J Immunol Methods 174: 83-93.
Park, J-H., von Maltzahn, G., Zhang, L., Derfus, A. M., Simberg, D., Harris, T. J., Bhatia, S, N., Ruoslahti, E., Sailor, M. J. Systematic Surface Engineering of Magnetic Nanoworms for in vivo Tumor Targeting. Small, in press.
Simberg, D., Duza T., Park, J. H., Essler M., Pilch, J., Zhang, L., Derfus A. M., Yang M., Hoffman R. M. Bhatia S., Sailor, M. J., and Ruoslahti, E. Biomimetic amplification of nanoparticle homing to tumors. Proc. Natl. Acad. Sci. USA 104: 932-936 (2007).

1. A composition comprising a surface molecule, one or more homing molecules, and a plurality of membrane perturbing molecules, wherein the homing molecule selectively homes to tumor vasculature.

2. The composition of claim, wherein one or more of the homing molecules comprise the amino acid sequence CGKRK (SEQ ID NO:1) or a conservative derivative thereof, the amino acid sequence CRKDKC (SEQ ID NO:2) or a conservative derivative thereof, or a combination.

3. The composition of claim 1, wherein one or more of the homing molecules comprise the amino acid sequence CGKRK (SEQ ID NO:1) or a conservative variant thereof.

4. The composition of claim 1, wherein one or more of the homing molecules comprise the amino acid sequence CGKRK (SEQ ID NO:1).

5. The composition of claim 1, wherein all of the one or more homing molecules comprise the amino acid sequence CGKRK (SEQ ID NO:1) or a conservative derivative thereof, the amino acid sequence CRKDKC (SEQ ID NO:2) or a conservative derivative thereof, or a combination.

6. The composition of claim 1, wherein one or more of the membrane perturbing molecules comprise the amino acid sequence D(KLAKLAK)₂ (SEQ ID NO:3) or a conservative variant thereof, (KLAKLAK)₂ (SEQ ID NO:3) or a conservative variant thereof, (KLAKKLA)₂ (SEQ ID NO:5) or a conservative variant thereof, (KAAKKAA)₂ (SEQ ID NO:6) or a conservative variant thereof, or (KLGKKLG)₃ (SEQ ID NO:7) or a conservative variant thereof, or a combination.

7. The composition of claim 1, wherein one or more of the membrane perturbing molecules comprise the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), or (KLGKKLG)₃ (SEQ ID NO:7), or a combination.

8. The composition of claim 1, wherein one or more of the membrane perturbing molecules comprise the amino acid sequence D(KLAKLAK)₂ (SEQ ID NO:3) or a conservative variant thereof.

9. The composition of claim 1, wherein one or more of the membrane perturbing molecules comprise the amino acid sequence _{D}(KLAKLAK)₂ (SEQ ID NO:3).

10. The composition of claim 1, wherein one or more of the membrane perturbing molecules are conjugated to one or more of the homing molecules.

11. The composition of claim 10, wherein one or more of the conjugated membrane perturbing molecules and homing molecules are covalently coupled.

12. The composition of claim 11, wherein one or more of the covalently coupled membrane perturbing molecules and homing molecules comprise fusion peptides.

13. The composition of claim 1, wherein the homing molecules are conjugated with the surface molecule.

14. The composition of claim 13, wherein one or more of the conjugated homing molecules are indirectly conjugated to the surface molecule.

15. The composition of claim 13, wherein one or more of the conjugated homing molecules are directly conjugated to the surface molecule.

16. The composition of claim 13, wherein one or more of the homing molecules are covalently coupled to the surface molecule.

17. The composition of claim 16, wherein one or more of the covalently coupled homing molecules are indirectly covalently coupled to the surface molecule.

18. The composition of claim 16, wherein one or more of the covalently coupled homing molecules are directly covalently coupled to the surface molecule.

19. The composition of claim 1, wherein the membrane perturbing molecules are conjugated with the surface molecule.

20. The composition of claim 19, wherein one or more of the conjugated membrane perturbing molecules are indirectly conjugated to the surface molecule.

21. The composition of claim 19, wherein one or more of the conjugated membrane perturbing molecules are directly conjugated to the surface molecule.

22. The composition of claim 19, wherein one or more of the membrane perturbing molecules are covalently coupled to the surface molecule.

23. The composition of claim 22, wherein one or more of the covalently coupled membrane perturbing molecules are indirectly covalently coupled to the surface molecule.

24. The composition of claim 22, wherein one or more of the covalently coupled membrane perturbing molecules are directly covalently coupled to the surface molecule.

25. The composition of claim 1, wherein one or more of the conjugated homing molecules are indirectly conjugated to the surface molecule via a linker, one or more of the conjugated membrane perturbing molecules are indirectly conjugated to the surface molecule via a linker, or both.

26. The composition of claim 1, wherein the composition further comprises a plurality of linkers.

27. The composition of claim 25, wherein at least one of the linkers comprises polyethylene glycol.

28. The composition of claim 1, wherein the composition further comprise one or more internalization elements.

29. The composition of claim 28, wherein one or more of the homing molecules comprise one or more of the internalization elements.

30. The composition of claim 28 era, wherein one or more of the membrane perturbing molecules comprise one or more of the internalization elements.

31. The composition of claim 28, wherein the surface molecule comprises one or more of the internalization elements not comprised in either the homing molecules or the membrane perturbing molecules.

32. The composition of claim 1, wherein the composition further comprise one or more tissue penetration elements.

33. The composition of claim 32, wherein one or more of the tissue penetration elements are comprised in an internalization element.

34. The composition of claim 32, wherein the tissue penetration element is a CendR element.

35. The composition of claim 1, wherein the composition binds inside tumor blood vessels.

36. The composition of claim 1, wherein the composition is internalized in cells.

37. The composition of claim 1, wherein the composition penetrates tissue.

38. The composition of claim 1, wherein the composition reduces tumor growth.

39. The composition of claim 1, wherein the surface molecule comprises an nanoparticle.

40. The composition of claim 1, wherein the surface molecule comprises a nanoworm.

41. The composition of claim 1, wherein the surface molecule comprises an iron oxide nanoworm.

42. The composition of claim 1, wherein the surface molecule comprises an iron oxide nanoparticle.

43. The composition of claim 1, wherein the surface molecule comprises an albumin nanoparticle.

44. The composition of claim 1, wherein the surface molecule comprises a liposome.

45. The composition of claim 1, wherein the surface molecule comprises a micelle.

46. The composition of claim 1, wherein the surface molecule comprises a phospholipid.

47. The composition of claim 1, wherein the surface molecule comprises a polymer.

48. The composition of claim 1, wherein the surface molecule comprises a microparticle.

49. The composition of claim 1, wherein the surface molecule comprises a fluorocarbon microbubble.

50. The composition of claim 1, wherein the composition comprises at least 100 homing molecules.

51. The composition of claim 50, wherein the composition comprises at least 1000 homing molecules.

52. The composition of claim 51, wherein the composition comprises at least 10,000 homing molecules.

53. The composition of claim 1, wherein the composition comprises at least 100 membrane perturbing molecules.

54. The composition of claim 53, wherein the composition comprises at least 1000 membrane perturbing molecules.

55. The composition of claim 54, wherein the composition comprises at least 10,000 membrane perturbing molecules.

56. The method of claim 1, wherein one or more of the homing molecules are modified homing molecules.

57. The composition of claim 56, wherein one or more of the homing molecules comprise a methylated homing molecule.

58. The composition of claim 57, wherein one or more of the methylated homing molecules comprise a methylated amino acid segment.

59. The method of claim 1, wherein one or more of the membrane perturbing molecules are modified membrane perturbing molecules.

60. The composition of claim 59, wherein one or more of the membrane perturbing molecules comprise a methylated membrane perturbing molecule.

61. The composition of claim 60, wherein one or more of the methylated membrane perturbing molecules comprise a methylated amino acid segment.

62. The composition of claim 56, wherein the amino acid sequence is N- or C-methylated in at least one position.

63. The composition of claim 1 further comprising one or more moieties.

64. The composition of claim 63, wherein the moieties are independently selected from the group consisting of an anti-angiogenic agent, a pro-angiogenic agent, a cancer chemotherapeutic agent, a cytotoxic agent, an anti-inflammatory agent, an anti-arthritic agent, a polypeptide, a nucleic acid molecule, a small molecule, an image contrast agent, a fluorophore, fluorescein, rhodamine, a radionuclide, indium-111, technetium-99, carbon-11, and carbon-13.

65. The composition of claim 63, wherein at least one of the moieties is a therapeutic agent.

66. The composition of claim 65, wherein the therapeutic agent is iRGD.

67. The composition of claim 65, wherein the therapeutic agent is Abraxane.

68. The composition of claim 65, wherein the therapeutic agent is paclitaxel.

69. The composition of claim 65, wherein the therapeutic agent is taxol.

70. The composition of claim 63, wherein at least one of the moieties is a detectable agent.

71. The composition of claim 70, wherein the detectable agent is FAM.

72. The composition of claim 1, wherein one or more of the homing molecules comprise the amino acid sequence CGKRK (SEQ ID NO:1), wherein one or more of the membrane perturbing molecules comprise the amino acid sequence _{D}(KLAKLAK)₂ (SEQ ID NO:3), wherein one or more of the conjugated homing molecules are indirectly conjugated to the surface molecule via a linker, and wherein one or more of the conjugated membrane perturbing molecules are indirectly conjugated to the surface molecule via a linker.

73. The composition of claim 72, wherein at least one of the linkers comprises polyethylene glycol.

74. A method comprising administering to a subject the composition of any one of claims 1-73, wherein the composition selectively homes to tumor vasculature in the subject, wherein the composition is internalized into cells at the site of the tumor vasculature.

75. The method of claim 74, wherein the composition has a therapeutic effect.

76. The method of claim 75, wherein the therapeutic effect is a slowing in the increase of or a reduction of tumor burden.

77. The method of claim 75, wherein the therapeutic effect is a slowing of the increase of or reduction of tumor size.

78. The method of claim 74, wherein the subject has one or more sites to be targeted, wherein the composition homes to one or more of the sites to be targeted.

79. The method of claim 74, wherein the subject has a tumor, wherein the composition has a therapeutic effect on the tumor.

80. The method of claim 74, wherein the composition penetrates tissue.

81. The method of claim 74, wherein the composition penetrates tumor tissue.


SEQUENCE LISTING (137 sequences)

SEQ ID NO: 1 - protein, 5 aa, Artificial Sequence
  chemically synthesized; homing peptide
CGKRK

SEQ ID NO: 2 - protein, 6 aa, Artificial Sequence
  chemically synthesized; homing peptide
CRKDKC

SEQ ID NO: 3 - protein, 14 aa, Artificial Sequence
  chemically synthesized; membrane perturbing molecule
KLAKLAKKLAKLAK

SEQ ID NO: 4 - protein, 9 aa, Artificial Sequence
  chemically synthesized; homing peptide
CRGDXGPXC

SEQ ID NO: 5 - protein, 14 aa, Artificial Sequence
  chemically synthesized; membrane perturbing molecule
KLAKKLAKLAKKLA

SEQ ID NO: 6 - protein, 14 aa, Artificial Sequence
  chemically synthesized; membrane perturbing molecule
KAAKKAAKAAKKAA

SEQ ID NO: 7 - protein, 21 aa, Artificial Sequence
  chemically synthesized; membrane perturbing molecule
KLGKKLGKLGKKLGKLGKKLG

SEQ ID NO: 8 - protein, 9 aa, Artificial Sequence
  chemically synthesized; brain homing peptide
CNSRLHLRC

SEQ ID NO: 9 - protein, 9 aa, Artificial Sequence
  chemically synthesized; brain homing peptide
CENWWGDVC

SEQ ID NO: 10 - protein, 21 aa, Artificial Sequence
  chemically synthesized; brain homing peptide
WRCVLREGPAGGCAWFNRHRL

SEQ ID NO: 11 - protein, 9 aa, Artificial Sequence
  chemically synthesized; brain homing peptide
CLSSRLDAC

SEQ ID NO: 12 - protein, 8 aa, Artificial Sequence
  chemically synthesized; brain homing peptide
CVLRGGRC

SEQ ID NO: 13 - protein, 9 aa, Artificial Sequence
  chemically synthesized; brain homing peptide
CNSRLQLRC

SEQ ID NO: 14 - protein, 7 aa, Artificial Sequence
  chemically synthesized; brain homing peptide
CGVRLGC

SEQ ID NO: 15 - protein, 8 aa, Artificial Sequence
  chemically synthesized; brain homing peptide
CKDWGRIC

SEQ ID NO: 16 - protein, 8 aa, Artificial Sequence
  chemically synthesized; brain homing peptide
CLDWGRIC

SEQ ID NO: 17 - protein, 8 aa, Artificial Sequence
  chemically synthesized; brain homing peptide
CTRITESC

SEQ ID NO: 18 - protein, 7 aa, Artificial Sequence
  chemically synthesized; brain homing peptide
CETLPAC

SEQ ID NO: 19 - protein, 8 aa, Artificial Sequence
  chemically synthesized; brain homing peptide
CRTGTLFC

SEQ ID NO: 20 - protein, 8 aa, Artificial Sequence
  chemically synthesized; brain homing peptide
CGRSLDAC

SEQ ID NO: 21 - protein, 9 aa, Artificial Sequence
  chemically synthesized; brain homing peptide
CRHWFDVVC

SEQ ID NO: 22 - protein, 8 aa, Artificial Sequence
  chemically synthesized; brain homing peptide
CANAQSHC

SEQ ID NO: 23 - protein, 8 aa, Artificial Sequence
  chemically synthesized; brain homing peptide
CGNPSYRC

SEQ ID NO: 24 - protein, 20 aa, Artificial Sequence
  chemically synthesized; brain homing peptide
YPCGGEAVAGVSSVRTMCSE

SEQ ID NO: 25 - protein, 20 aa, Artificial Sequence
  chemically synthesized; brain homing peptide
LNCDYQGTNPATSVSVPCTV

SEQ ID NO: 26 - protein, 7 aa, Artificial Sequence
  chemically synthesized; kidney homing peptide
CLPVASC

SEQ ID NO: 27 - protein, 7 aa, Artificial Sequence
  chemically synthesized; kidney homing peptide
CGAREMC

SEQ ID NO: 28 - protein, 8 aa, Artificial Sequence
  chemically synthesized; kidney homing peptide
CKGRSSAC

SEQ ID NO: 29 - protein, 8 aa, Artificial Sequence
  chemically synthesized; kidney homing peptide
CWARAQGC

SEQ ID NO: 30 - protein, 8 aa, Artificial Sequence
  chemically synthesized; kidney homing peptide
CLGRSSVC

SEQ ID NO: 31 - protein, 8 aa, Artificial Sequence
  chemically synthesized; kidney homing peptide
CTSPGGSC

SEQ ID NO: 32 - protein, 8 aa, Artificial Sequence
  chemically synthesized; kidney homing peptide
CMGRWRLC

SEQ ID NO: 33 - protein, 8 aa, Artificial Sequence
  chemically synthesized; kidney homing peptide
CVGECGGC

SEQ ID NO: 34 - protein, 7 aa, Artificial Sequence
  chemically synthesized; kidney homing peptide
CVAWLNC

SEQ ID NO: 35 - protein, 7 aa, Artificial Sequence
  chemically synthesized; kidney homing peptide
CRRFQDC

SEQ ID NO: 36 - protein, 7 aa, Artificial Sequence
  chemically synthesized; kidney homing peptide
CLMGVHC

SEQ ID NO: 37 - protein, 8 aa, Artificial Sequence
  chemically synthesized; kidney homing peptide
CKLLSGVC

SEQ ID NO: 38 - protein, 8 aa, Artificial Sequence
  chemically synthesized; kidney homing peptide
CFVGHDLC

SEQ ID NO: 39 - protein, 7 aa, Artificial Sequence
  chemically synthesized; kidney homing peptide
CRCLNVC

SEQ ID NO: 40 - protein, 7 aa, Artificial Sequence
  chemically synthesized; kidney homing peptide
CKLMGEC

SEQ ID NO: 41 - protein, 9 aa, Artificial Sequence
  chemically synthesized; skin homing peptide
CARSKNKDC

SEQ ID NO: 42 - protein, 6 aa, Artificial Sequence
  chemically synthesized; skin homing peptide
CRKDKC

SEQ ID NO: 43 - protein, 13 aa, Artificial Sequence
  chemically synthesized; skin homing peptide
CVALCREACGEGC

SEQ ID NO: 44 - protein, 13 aa, Artificial Sequence
  chemically synthesized; skin homing peptide
CSSGCSKNCLEMC

SEQ ID NO: 45 - protein, 8 aa, Artificial Sequence
  chemically synthesized; skin homing peptide
CIGEVEVC

SEQ ID NO: 46 - protein, 9 aa, Artificial Sequence
  chemically synthesized; skin homing peptide
CKWSRLHSC

SEQ ID NO: 47 - protein, 9 aa, Artificial Sequence
  chemically synthesized; skin homing peptide
CWRGDRKIC

SEQ ID NO: 48 - protein, 9 aa, Artificial Sequence
  chemically synthesized; skin homing peptide
CERVVGSSC

SEQ ID NO: 49 - protein, 9 aa, Artificial Sequence
  chemically synthesized; skin homing peptide
CLAKENVVC

SEQ ID NO: 50 - protein, 13 aa, Artificial Sequence
  chemically synthesized; lung homing peptide
CGFECVRQCPERC

SEQ ID NO: 51 - protein, 8 aa, Artificial Sequence
  chemically synthesized; lung homing peptide
CGFELETC

SEQ ID NO: 52 - protein, 8 aa, Artificial Sequence
  chemically synthesized; lung homing peptide
CTLRDRNC

SEQ ID NO: 53 - protein, 8 aa, Artificial Sequence
  chemically synthesized; lung homing peptide
CIGEVEVC

SEQ ID NO: 54 - protein, 8 aa, Artificial Sequence
  chemically synthesized; lung homing peptide
CTLRDRNC

SEQ ID NO: 55 - protein, 8 aa, Artificial Sequence
  chemically synthesized; lung homing peptide
CGKRYRNC

SEQ ID NO: 56 - protein, 8 aa, Artificial Sequence
  chemically synthesized; lung homing peptide
CLRPYLNC

SEQ ID NO: 57 - protein, 12 aa, Artificial Sequence
  chemically synthesized; lung homing peptide
CTVNEAYKTRMC

SEQ ID NO: 58 - protein, 12 aa, Artificial Sequence
  chemically synthesized; lung homing peptide
CRLRSYGTLSLC

SEQ ID NO: 59 - protein, 12 aa, Artificial Sequence
  chemically synthesized; lung homing peptide
CRPWHNQAHTEC

SEQ ID NO: 60 - protein, 9 aa, Artificial Sequence
  chemically synthesized; pancreas homing peptide
SWCEPGWCR

SEQ ID NO: 61 - protein, 7 aa, Artificial Sequence
  chemically synthesized; pancreas homing peptide
CKAAKNK

SEQ ID NO: 62 - protein, 7 aa, Artificial Sequence
  chemically synthesized; pancreas homing peptide
CKGAKAR

SEQ ID NO: 63 - protein, 8 aa, Artificial Sequence
  chemically synthesized; pancreas homing peptide
VGVGEWSV

SEQ ID NO: 64 - protein, 7 aa, Artificial Sequence
  chemically synthesized; intestine homing peptide
YSGKWGW

SEQ ID NO: 65 - protein, 7 aa, Artificial Sequence
  chemically synthesized; uterus homing peptide
GLSGGRS

SEQ ID NO: 66 - protein, 7 aa, Artificial Sequence
  chemically synthesized; adrenal gland homing peptide
LMLPRAD

SEQ ID NO: 67 - protein, 7 aa, Artificial Sequence
  chemically synthesized; adrenal gland homing peptide
LPRYLLS

SEQ ID NO: 68 - protein, 9 aa, Artificial Sequence
  chemically synthesized; retina homing peptide
CSCFRDVCC

SEQ ID NO: 69 - protein, 9 aa, Artificial Sequence
  chemically synthesized; retina homing peptide
CRDVVSVIC

SEQ ID NO: 70 - protein, 7 aa, Artificial Sequence
  chemically synthesized; gut homing peptide
YSGKWGK

SEQ ID NO: 71 - protein, 8 aa, Artificial Sequence
  chemically synthesized; gut homing peptide
GISALVLS

SEQ ID NO: 72 - protein, 7 aa, Artificial Sequence
  chemically synthesized; gut homing peptide
SRRQPLS

SEQ ID NO: 73 - protein, 7 aa, Artificial Sequence
  chemically synthesized; gut homing peptide
MSPQLAT

SEQ ID NO: 74 - protein, 7 aa, Artificial Sequence
  chemically synthesized; gut homing peptide
MRRDEQR

SEQ ID NO: 75 - protein, 7 aa, Artificial Sequence
  chemically synthesized; gut homing peptide
QVRRVPE

SEQ ID NO: 76 - protein, 7 aa, Artificial Sequence
  chemically synthesized; gut homing peptide
VRRGSPQ

SEQ ID NO: 77 - protein, 7 aa, Artificial Sequence
  chemically synthesized; gut homing peptide
GGRGSWE

SEQ ID NO: 78 - protein, 7 aa, Artificial Sequence
  chemically synthesized; gut homing peptide
FRVRGSP

SEQ ID NO: 79 - protein, 7 aa, Artificial Sequence
  chemically synthesized; gut homing peptide
RVRGPER

SEQ ID NO: 80 - protein, 7 aa, Artificial Sequence
  chemically synthesized; liver homing peptide
VKSVCRT

SEQ ID NO: 81 - protein, 7 aa, Artificial Sequence
  chemically synthesized; liver homing peptide
WRQNMPL

SEQ ID NO: 82 - protein, 7 aa, Artificial Sequence
  chemically synthesized; liver homing peptide
SRRFVGG

SEQ ID NO: 83 - protein, 7 aa, Artificial Sequence
  chemically synthesized; liver homing peptide
ALERRSL

SEQ ID NO: 84 - protein, 7 aa, Artificial Sequence
  chemically synthesized; liver homing peptide
ARRGWTL

SEQ ID NO: 85 - protein, 7 aa, Artificial Sequence
  chemically synthesized; prostate homing peptide
SMSIARL

SEQ ID NO: 86 - protein, 7 aa, Artificial Sequence
  chemically synthesized; prostate homing peptide
VSFLEYR

SEQ ID NO: 87 - protein, 7 aa, Artificial Sequence
  chemically synthesized; prostate homing peptide
RGRWLAL

SEQ ID NO: 88 - protein, 7 aa, Artificial Sequence
  chemically synthesized; ovary homing peptide
EVRSRLS

SEQ ID NO: 89 - protein, 7 aa, Artificial Sequence
  chemically synthesized; ovary homing peptide
VRARLMS

SEQ ID NO: 90 - protein, 7 aa, Artificial Sequence
  chemically synthesized; ovary homing peptide
RVGLVAR

SEQ ID NO: 91 - protein, 7 aa, Artificial Sequence
  chemically synthesized; ovary homing peptide
RVRLVNL

SEQ ID NO: 92 - protein, 5 aa, Artificial Sequence
  chemically synthesized; clot binding/homing peptide
CREKA

SEQ ID NO: 93 - protein, 5 aa, Artificial Sequence
  chemically synthesized; heart homing peptide
CRPPR

SEQ ID NO: 94 - protein, 9 aa, Artificial Sequence
  chemically synthesized; heart homing peptide
CGRKSKTVC

SEQ ID NO: 95 - protein, 6 aa, Artificial Sequence
  chemically synthesized; heart homing peptide
CARPAR

SEQ ID NO: 96 - protein, 6 aa, Artificial Sequence
  chemically synthesized; heart homing peptide
CPKRPR

SEQ ID NO: 97 - protein, 6 aa, Artificial Sequence
  chemically synthesized; heart homing peptide
CKRAVR

SEQ ID NO: 98 - protein, 9 aa, Artificial Sequence
  chemically synthesized; heart homing peptide
CRNSWKPNC

SEQ ID NO: 99 - protein, 5 aa, Artificial Sequence
  chemically synthesized; heart homing peptide
RGSSS

SEQ ID NO: 100 - protein, 9 aa, Artificial Sequence
  chemically synthesized; heart homing peptide
CRSTRANPC

SEQ ID NO: 101 - protein, 9 aa, Artificial Sequence
  chemically synthesized; heart homing peptide
CPKTRRVPC

SEQ ID NO: 102 - protein, 9 aa, Artificial Sequence
  chemically synthesized; heart homing peptide
CSGMARTKC

SEQ ID NO: 103 - protein, 7 aa, Artificial Sequence
  chemically synthesized; heart homing peptide
GGGVFWQ

SEQ ID NO: 104 - protein, 7 aa, Artificial Sequence
  chemically synthesized; heart homing peptide
HGRVRPH

SEQ ID NO: 105 - protein, 7 aa, Artificial Sequence
  chemically synthesized; heart homing peptide
VVLVTSS

SEQ ID NO: 106 - protein, 8 aa, Artificial Sequence
  chemically synthesized; heart homing peptide
CLHRGNSC

SEQ ID NO: 107 - protein, 12 aa, Artificial Sequence
  chemically synthesized; heart homing peptide
CRSWNKADNRSC

SEQ ID NO: 108 - protein, 9 aa, Artificial Sequence
  chemically synthesized; heart homing peptide
CGRKSKTVC

SEQ ID NO: 109 - protein, 6 aa, Artificial Sequence
  chemically synthesized; heart homing peptide
CKRAVR

SEQ ID NO: 110 - protein, 9 aa, Artificial Sequence
  chemically synthesized; heart homing peptide
CRNSWKPNC

SEQ ID NO: 111 - protein, 9 aa, Artificial Sequence
  chemically synthesized; heart homing peptide
CPKTRRVPC

SEQ ID NO: 112 - protein, 9 aa, Artificial Sequence
  chemically synthesized; heart homing peptide
CSGMARTKC

SEQ ID NO: 113 - protein, 6 aa, Artificial Sequence
  chemically synthesized; heart homing peptide
CARPAR

SEQ ID NO: 114 - protein, 6 aa, Artificial Sequence
  chemically synthesized; heart homing peptide
CPKRPR

SEQ ID NO: 115 - protein, 5 aa, Artificial Sequence
  chemically synthesized; tumor blood vessel homing peptide
CNGRC

SEQ ID NO: 116 - protein, 9 aa, Artificial Sequence
  chemically synthesized; homing peptide
CSRPRRSEC

SEQ ID NO: 117 - protein, 9 aa, Artificial Sequence
  chemically synthesized; homing peptide
CSRPRRSVC

SEQ ID NO: 118 - protein, 9 aa, Artificial Sequence
  chemically synthesized; homing peptide
CSRPRRSWC

SEQ ID NO: 119 - protein, 31 aa, Artificial Sequence
  chemically synthesized; homing peptide
KDEPQRRSARLSAKPAPPKPEPKPKKAPAKK

SEQ ID NO: 120 - protein, 10 aa, Artificial Sequence
  chemically synthesized; homing peptide
PQRRSARLSA

SEQ ID NO: 121 - protein, 10 aa, Artificial Sequence
  chemically synthesized; homing peptide
PKRRSARLSA

SEQ ID NO: 122 - protein, 7 aa, Artificial Sequence
  chemically synthesized; homing peptide
CRGRRST

SEQ ID NO: 123 - protein, 9 aa, Artificial Sequence
  chemically synthesized; tumor homing peptide
CARSKNKDC

SEQ ID NO: 124 - protein, 6 aa, Artificial Sequence
  chemically synthesized; tumor homing peptide
CRKDKC

SEQ ID NO: 125 - protein, 10 aa, Artificial Sequence
  chemically synthesized; tumor homing peptide
CGLIIQKNEC

SEQ ID NO: 126 - protein, 10 aa, Artificial Sequence
  chemically synthesized; tumor homing peptide
CNAGESSKNC

SEQ ID NO: 127 - protein, 9 aa, Artificial Sequence
  chemically synthesized; tumor homing peptide
CGNKRTRGC

SEQ ID NO: 128 - protein, 9 aa, Artificial Sequence
  chemically synthesized; tumor homing peptide
CARSKNKDC

SEQ ID NO: 129 - protein, 6 aa, Artificial Sequence
  chemically synthesized; homing peptide
CRKDKC

SEQ ID NO: 130 - protein, 4 aa, Artificial Sequence
  chemically synthesized; internalization sequence
XXXX

SEQ ID NO: 131 - protein, 5 aa, Artificial Sequence
  chemically synthesized; internalization sequence
XXXKG

SEQ ID NO: 132 - protein, 9 aa, Artificial Sequence
  chemically synthesized; tumor homing peptide
CNRRTKAGC

SEQ ID NO: 133 - protein, 6 aa, Artificial Sequence
  chemically synthesized; tumor homing peptide
CRSRKG

SEQ ID NO: 134 - protein, 9 aa, Artificial Sequence
  chemically synthesized; tumor homing peptide
CRGDKGPDC

SEQ ID NO: 135 - protein, 5 aa, Artificial Sequence
  chemically synthesized; control peptide
KAKEC

SEQ ID NO: 136 - protein, 5 aa, Artificial Sequence
  chemically synthesized; homing peptide
CRGDC

SEQ ID NO: 137 - protein, 34 aa, Artificial Sequence
  chemically synthesized; tumor homing peptide
AKVKDEPQRRSARLSAKPAPPKPEPKPKKAPAKK